(19) United States
(12) Patent Application Publication
(10) Pub. No.: US 2004/0121350 A1
(43) Pub. Date: Jun. 24, 2004

(54) System and method for identifying a panel of indicators

(75) Inventors: Joseph Michael Anderberg, Encinitas, CA (US); Kenneth F. Buechler, Rancho Santa Fe, CA (US); Paul H. McPherson, Encinitas, CA (US); Howard J. Kirchick, San Diego, CA (US); Jeffrey R. Dahlen, San Diego, CA (US)

Correspondence Address: FOLEY & LARDNER, P.O. BOX 80278, SAN DIEGO, CA, 92138-0278, US

(73) Assignee: Biosite Incorporated

(21) Appl. No.: 10/410,572

(22) Filed: Apr. 8, 2003

Related U.S. Application Data
(60) Provisional application No. 60/436,392, filed on Dec. 24, 2002.

(51) Int. Cl. C12Q 1/68; G01N 33/53; G06F 19/00; G01N 33/48; G01N 33/50

(52) U.S. Cl. 435/6; 435/7.1; 702/19

(57) ABSTRACT

The present invention relates to methods and system for the diagnosis diseases or conditions. In a particular aspect, a disclosed method for determining a panel includes calculating a panel response for each patient in a set of diseased patients and in a set of non-diseased patients. The panel response is a function of the value of each of a plurality of markers in a panel of markers. The method also includes calculating a value for an objective function. The objective function is indicative of the effectiveness of the panel. The steps of calculating a panel response for each patient and calculating a value for an objective function are iterated by varying at least one of the parameters relating to the panel response function and a sense of each marker to facilitate optimization of the objective function. The objective function may be a measure of an overlap of panel responses of diseased patients and panel responses of non-diseased patients. The contribution of each marker to the objective function may be determined, and the panel size may be reduced by removing the poorest markers. Thus, an optimum panel of markers and an optimal panel response function for the diagnosis of a disease or condition may be determined.

This application is related to U.S. Provisional Patent Application No. 60/436,692 (Atty Docket No. 071949-6801, Express Mail No. EV 003428575 US), filed Dec. 24, 2002, from which priority is claimed, and which is hereby incorporated by reference in its entirety, including all tables, figures, and claims.

### FIELD OF THE INVENTION

The present invention relates to the identification and use of diagnostic markers for various diseases or conditions. More particularly, the invention relates to methods and systems for identifying and utilizing panel of markers for detection of one or more particular diseases or conditions.

### BACKGROUND OF THE INVENTION

The background of the invention is provided to aid the reader in understanding the invention and is not admitted to describe or constitute prior art to the present invention.

The clinical presentation of certain diseases can often be strikingly similar, even though the underlying diseases, and the appropriate treatments to be given to one suffering from the various diseases, can be completely distinct. For example, subjects may present in an urgent care facility exhibiting a deceptively simple constellation of apparent symptoms (e.g., fever, shortness of breath, dizzyness, headache) that may be characteristic of a variety of unrelated conditions. Diagnostic methods often involve the comparison of symptoms and/or diagnostic test results known to be associated with one or more diseases that exhibit a similar clinical presentation to the symptoms and/or diagnostic results exhibited by the subject, in order to identify the underlying disease or condition present in the subject.

The acuteness or severity of the symptoms often dictates how rapidly a diagnosis must be established and treatment initiated. For example, immediate diagnosis and care of a patient experiencing a variety of acute conditions can be critical. See, e.g., Harris, Aust. Fam. Physician 31: 802-06 (2002) (asthma); Goldhaber, Eur. Respir. J. Suppl. 35:22s-27s (2002) (pulmonary embolism); Lundergan et al., Am. Heart J. 144: 456-62 (2002) (myocardial infarction). However, even in cases where the apparent symptoms appear relatively stable, rapid diagnosis, and the rapid initiation of treatment, can provide both relief from immediate discomfort and advantageous improvement in prognosis.

Recently, workers seeking to provide rapid diagnostic methods for various diseases or conditions have sought to identify “markers” for diseases; that is, molecules that are present in a sample obtained from a subject suffering from a disease of interest in an amount that differs from the amount present in a sample from a “normal,” non-diseased subject.

Diagnoses of many diseases or conditions, such as cardiovascular disease and stroke, for example, are performed by measurement of the levels of particular markers in a patient. Often, however, a single marker is generally incapable of providing clinical utility because its value does not provide a means of confidently distinguishing between a diseased patient and a non-diseased patient.

As an example, FIG. 1 illustrates that the levels of a particular marker expressed in a diseased and a non-diseased population. As shown in the figure, the marker levels in these two populations may be distributed over broad ranges in a distribution pattern. Although the diseased population in this example generally may exhibits higher or lower levels for the marker than the non-diseased population, substantial portions of each population fall within a region of overlapping values. Thus, definitive or confident diagnosis of a disease or a condition based on the measurement of this single marker may be impossible. Traditionally one chooses a cutoff value in the overlap region. The cutoff is chosen to optimize the number of false positive versus the number of false negatives. In practice physicians often treat a patient based on where they fall relative to the cutoff. They often do not consider how close the patient is to the cutoff.

The effectiveness of a test having such an overlap is often expressed using a ROC (Receiver Operating Characteristic) curve. Other measures, such as positive predictive value (PPV) and negative predictive value (NPV) may also be used as a measure of the effectiveness of the test. ROC curves are well known to those skilled in the art. Thus, the details pertaining to ROC curves are beyond the scope of this document, however there is a brief description below. Further, reference may be made to Zweig, MH. & Campbell, C. C., Clin Chem 39, 561-577 (1993) and Hendrson, A. R., Ann. Clin. Biochem 30, 521-539 (1993).

FIG. 3 illustrates an example of a ROC curve for the marker level distributions of FIG. 1. The ROC curve shows the trade off between the sensitivity and specificity of a marker. The sensitivity is a measure of the ability of the marker to detect the disease, and the specificity is a measure of the ability of the marker to detect the absence of the disease. The horizontal axis of the ROC curve represents (1-specificity), which increases with the rate of false positives. The vertical axis of the curve represents sensitivity, which increases with the rate of true positives. Thus, for a particular cutoff selected, the values of specificity and sensitivity may be determined. The right hand end of the curve is the minimum cutoff, the left hand end of the curve is the maximum cutoff. As the cutoff is changed to increase specificity, sensitivity usually is reduced and vice versa. The area under the ROC curve is a measure of the utility of the measured marker level in the correct identification of one or more diseases or conditions. Thus, the area under the ROC curve can be used to determine the effectiveness of the test. Note the area is independent of the cutoff value.

Panels of multiple markers may improve the likelihood of an accurate diagnosis. The multiple marker “panel” for a particular disease is preferably selected such that a particular “profile” of marker levels is specific for that disease and capable of clearly distinguishing disease from non-disease. However, methods for identifying such panels, and the particular “profiles” that provide clinical utility, are typically empirical in nature, relying on trial-and-error. Furthermore, because the computational complexity involved in identifying suitable diagnostic thresholds and/or profiles increases as the number of markers in a potential panel increase, marker panels typically involve only a few markers. Searching for an effective panel from among a large number of markers can become the computational equivalent of finding a needle in a haystack. For example, often one might look for elevation of 4 of 6 markers, or more generally n of m markers, to define a positive state. In this example the cutoff values for each marker are chosen, then the data analyzed to see how effective the test is. This is repeated for different number of elevated markers, cutoffs and markers. In this example, all markers are treated with equal importance, there is no method to adjust the relative importance.

### BRIEF SUMMARY OF THE INVENTION

The method disclosed in this document provides a means to systematically find the optimal markers and panels of markers to distinguish (compare) non-disease from disease, and it also optimizes the way in which the marker values are used. A first step to simplify the problem of defining a marker or a panel of markers is defining an ‘objective function’. An objective function is a scalar function, and will represent the effectiveness of the test for diagnosis of non-disease from disease. For example, rather than requiring n elevated markers to define a positive state and then quantifying the effectiveness of this algorithm, one can generate a ROC curve from the number of elevated markers, and use the area under the ROC curve (“the ROC curve area”) to define the effectiveness of the test. By using the ROC curve area as the effectiveness of the test, the optimization problem has been simplified. This is because the search space has been reduced since there is no need to calculate the effectiveness associated with each of the m values for n elevated markers. In this example, the number of elevated markers can be thought of as a concentration for the ROC curve, but as described above, the selection of the cutoff concentration is not required to determine if a test will be effective. Another step to simplify the problem of defining a marker or a panel of markers may be to define a systematic way to find the best way to use the markers. Without this it is very difficult to find the best markers because one needs to distinguish the markers and how to use them. A systematic method to find the best way to use the markers is to combine all the values into one result, the “panel response”. Functional forms of the panel response can be selected. Once this is done search routines can be employed to find the panel response function to maximize or minimize the objective function for a set of markers.

The method may also includes a technique for determining the relative importance of the markers in the set, and subsequently determine the optimum markers to use, for example, in a panel of n markers.

In addition to measured marker levels, other information including a patient's history, sex, age, race, and other factors may also require consideration. In this regard, embodiments of the disclosed method may accommodate such factors as markers.

Specifically, certain disclosed embodiments of the present invention relate to the identification and use of diagnostic markers for cardiac diseases and stroke and cerebral injury. Generally, the methods and systems described herein can meet the need in the art for the development of an effective panel of markers for the accurate diagnosis of a selected disease or condition. More generally, the disclosed methods and systems may be used to develop criteria for distinguishing members of two or more groups for whom the distribution of certain characteristics are known.

In a first aspect, the invention discloses a method of identifying a panel of markers for diagnosis of a disease or a condition. The method includes calculating a panel response for each patient in a set of diseased patients and in a set of non-diseased patients. The panel response is a function of value of each of a plurality of markers in a panel of markers.

The term “panel” as used herein refers to a set of markers. The panel may include any practical number of markers appropriate for use with the diagnosis of the particular one or more diseases or conditions.

The term “marker” as used herein refers to proteins, polypeptides, nucleic acids, bacteria, viruses, prions, small molecules and the like, to be used as targets for screening test samples obtained from subjects. “Proteins, polypeptides, or small molecules” used as markers in the present invention are contemplated to include any fragments thereof, in particular, immunologically detectable fragments. “Marker”, as used herein, may include derived markers as defined below, and may also include such characteristics as patient's history, age, sex and race, for example. Certain markers are also known in the field as “analytes”. A marker is said to be a specific marker of the disease if only the presence or absence of the target disease condition influences its value. A marker is said to be a nonspecific marker of the disease if many disease conditions influence its value. An example of a specific marker is TnI, which, when elevated above about 1 ng/ml is specific to myocardial infarction. An example of a non specific marker is CRP, which is elevated in conditions that promote the inflamatory repsonse.

The phrase “diagnosis” as used herein refers to methods by which the skilled artisan can estimate and/or determine whether or not a patient is suffering from a given disease or condition. The skilled artisan often makes a diagnosis on the basis of one or more diagnostic markers, the presence, absence, or amount of which may be indicative of the presence, severity, or absence of the condition. In addition to markers, other tests, such as ECG, Echo, and MRI, and other factors, such as patient's history, sex, age, and race, may also be used in making the diagnosis. As used herein, the term “markers” also includes these other tests and other factors.

The term “panel response” as used herein refers to a scalar function or its value, which is a function of the marker values of the panel. Most generally, the panel response is a function of the marker values (M₁₋ₙ), written as PR=f (M₁₋ₙ). In a preferred embodiment the panel response is a summation over indicator values (I) of each marker. The indicator value is generally a function of the marker value. This can be represented as

$$PR = {\sum }_{Marker s} {I}_{i} ( {M}_{i} ) \cdot {W}_{i} ,$$

where Iᵢ is a function of the marker value Mᵢ, Wᵢ is a weighting coefficient that scales the indicator function. For definitive purposes, in this document it will be assumed that the panel response is scaled such that all values are between 0 and 1, but other increments can apply.

The set of diseased patients and set of non-diseased patients' may include patients whose state, whether diseased or non-diseased, has been confirmed and for whom marker levels are available for one or more markers.

The term “marker value” as used herein refers to a numeric value, such as a value representing the result of an assay of the marker. For example, the marker value may be expressed in units of concentration or number. When the marker represents characteristics such as a patient's history, then the value may be a numeric representation, or mapping, of the history information.

The term “derived marker” as used herein refers to a value that is a function of one or more measured markers. For example, derived markers may be related to the change over a time interval in one or more measured marker values, may be related to a ratio of measured marker values, may be a marker value at a different measurement time, or may be a complex function such as a panel response function.

The method further comprises calculating a value for an objective function, the objective function being indicative of an effectiveness of the panel.

The term “objective function” as used herein refers to a scalar function or its value, which may be a function of the plurality of panel responses and known disease states or diagnoses of a collection of patient samples. The objective function is a measure of the clinical effectiveness of the test, or the ability to distinguish disease from non-disease. An example of an objective function is the area under the ROC curve. The objective function may be related to the amount of overlap between the diseased and non-diseased panel response values. The objective function is a scalar value, which is indicative of the effectiveness of the panel. The objective function may be defined by a user as a function of various outputs, such as ROC curve features defined below, of the panel responses for the groups of patients.

The method of the first aspect of the invention also comprises iterating the calculating a panel response for each patient and calculating a value for an objective function by varying at least one of parameters relating to the panel response function and a sense of each marker to facilitate optimization of the objective function.

“Iterating” may include repeating the steps with variations in the inputs, where the variations may be dependant on the outputs of the previous iteration. “Varying” may include tweaking a parameter by either a predetermined amount, an amount dependant on an output of the previous iteration or a random amount.

The term “sense” as used herein refers to the direction of the response of a marker with disease state. If a marker value is elevated in diseased patients relative to non-diseased patients, then the marker is said to have a positive sense. If the marker value is lower in diseased patients relative to non-diseased patients then the marker is said to have a negative sense. If the probability of a finding the marker value near some specific value is elevated in diseased patients relative to non-diseased patients, the sense is said to be positive. If the probability of a finding a marker value near some specific value is reduced in diseased patients relative to non-diseased patients, the sense is said to be negative. One skilled in the art will recognize that it is trivial to invert functions or map the marker value such that a negative sense marker can be analyzed in the same way as a positive sense marker. Throughout this document the marker sense is described as positive. This is for conciseness only, all concepts and claims can apply to both negative and positive sense markers, and both positive and negative senses are implicitly included.

The term “parameters” as used herein refers to coefficients, powers, etc. of a function that may be varied to modify the functional value. For example, if the function is a ramp function, the low threshold and the high threshold, may be two parameters that are varied. If the function is a Gaussian the width and location may be two parameters that are varied. The optimization process will modify one or more of the parameters of the panel response function, which in one embodiment may include all of the parameters of the used indicator functions and weighting coefficients.

According to another aspect of the invention, a system for identifying a panel of markers for diagnosis of a disease or a condition includes means for calculating a panel response for each patient in a set of diseased patients and in a set of non-diseased patients. In one embodiment the panel response is a function of a value of each of a plurality of markers in a panel of markers. The means for calculating may be a central processing unit (CPU), as may be available on a desktop computer, a laptop computer, a workstation or a mainframe, for example.

The system further includes means for calculating a value for an objective function. The objective function is indicative of the effectiveness of the panel. In certain embodiments, an objective function may be a measure of overlap of panel responses of diseased patients and panel responses of non-diseased patients.

Further, the system includes means for iteratively activating the means for calculating a panel response and the means for calculating a value for an objective function, by varying at least one of the following parameters to facilitate optimization of said objective function: parameters relating to the panel response function and a sense of each marker.

In another aspect of the invention, a program product includes machine readable program code for causing a machine to perform certain method steps. The method steps include calculating a panel response for each patient in a set of diseased patients and in a set of non-diseased patients. The panel response is a function of value of each of a plurality of markers in a panel of markers.

The method steps further include calculating a value for an objective function. The objective function is indicative of the effectiveness of the panel. Further, the method steps include iterating the steps of calculating a panel response for each patient and calculating a value for an objective function by varying at least one of the following parameters to facilitate optimization of said objective function: parameters relating to the panel response function and a sense of each marker.

In a preferred embodiment, the program product includes machine readable code embedded in a portable meter. The term “portable meter,” as used herein, may include any number of devices having the ability to execute coded instructions. In a further preferred embodiment, the portable meter is a fluorometer. In an alternate embodiment, the portable meter is a reflectometer.

In a preferred embodiment, the program product includes machine readable code embedded in a computer. In a further preferred embodiment, the computer is a portable computer. In another preferred embodiment, the computer is adapted to be accessed through a network, such as a public network like the Internet.

In another preferred embodiment, the computer is adapted to be coupled to an analyzer. In a further preferred embodiment, the analyzer is an immunoassay analyzer. In an alternate embodiment, the analyzer is a single nucleotide polymorphism detector. In another embodiment, the analyzer is adapted to sort and count similar and different particles and cells.

In a preferred embodiment, the panel response is a function of the value of an indicator for each of a plurality of markers in a panel of markers and a weighting coefficient for each marker. The indicator is a mapping, for each of the plurality of markers, of marker levels. The mapping is according to an indicator function. The iterating includes varying at least one of the weighting coefficients, parameters relating to the indicator function, and a sense of each marker to facilitate optimization of the objective function.

The term “indicator function” as used herein refers to a scalar function or its value, which is a function of a marker value. The mapping is in accordance with a indicator function. The indicator function may be any function providing a value dependent on the marker level. The indicator function may be a mapping of marker values into values that may be more closely related to the probability of diseased state at that marker value. The indicator function may be scaled such that all values are between 0 and 1. In this document it will be assumed that the indicator function is scaled such that all values are between 0 and 1. This scaling does not influence the result of the method, however in practice it does simplify some formulations. For example, to change a positive indicator function (PIF) to work with a negative sense marker the negative indicator function (NIF) may be defined as NIF=1−PIF.

The term “mapping” as used herein refers to a relation between a value in one domain to a value in another domain. The mapping relation may be a one-to-one relationship or a one-to-many relationship.

The term “elevation indicator function” as used herein refers to a scalar function that has a high and monotonic rate of change between low and high threshold values, and a smaller rate of change elsewhere. Examples of this type of function include step, ramp, ‘S’ or sigmoid functions. One skilled in the art will recognize that there are many such functions.

The term “localization indicator function” as used herein refers to a scalar function that is peaked near some expected value, and decreases when the marker value is further away from the expected value. Examples of this type of function include triangle, square, trapezoid, or Gaussian functions. One skilled in the art will recognize that there are many such functions.

The term “contribution” as used herein refers to the relative amount that a marker contributes to the objective function. The contribution may be related to the importance of a marker.

The term “test” as used herein refers to a method performed which yields an output related to a clinical outcome. A test may comprise values of 1 or more markers. A test may also be a procedure used in the determination of a panel response. Commonly, a test is also an immunoassay.

In the method the marker values may be combined into one value, the panel response. As described above, in a preferred embodiment the panel response is represented as

$$PR = {\sum }_{Marker s} {I}_{i} ( {M}_{i} ) \cdot {W}_{i} .$$

Choosing different functional forms for the indicator I changes the way a marker is used. For example, when several nonspecific markers are used, then combined elevations of the markers may indicate a diseased state. The appropriate indicator functions could be elevation indicator functions as defined above. In this example, when the marker value is below a low threshold then there is little or no change in the indicator function with marker value, and when above a high threshold than again there is little or no change in indicator value. Between these thresholds the indicator value increases or decreases with marker value. One skilled in the art will recognize there are many functions that have this property. Physically one can associate the thresholds with the lower and upper end of the overlap region as illustrated in FIG. 4.

In another embodiment the indicator function is chosen to localize the marker value. For example if a certain pattern of marker levels is associated with a disease state then the indicator function could be a localization indicator function as defined above. These functions give a high response when the marker is near the optimal value. One skilled in the art will recognize there are many functions that have this localization property. In an example using these functions, certain disease states such as unstable angina, may be an intermediate disease. A marker such as TnI is elevated by ischemia associated with unstable angina, but is elevated still further by necrosis associated with myocardial infarction. Other markers may be elevated with unstable angina, but not elevated with myocardial infarction. The indicator function of each analyte can be different so panels can consist of markers of both types, as needed in the example above. A panel response may be a numerical value for each patient. The range of values of the panel response may be set to any desired range. For example, the values of the panel response may be scaled to fall between zero and one.

In a preferred embodiment the method includes utilizing a search engine to find optimal parameters for the panel response function. The search engine is able to efficiently vary parameters of the panel response until it finds a set that results in a local maximization of the objective function. Because the objective function is a measure of the effectiveness of the test, the optimized panel response may provide an improved diagnostic value.

In a preferred embodiment the method includes calculating a contribution for each marker. In another preferred embodiment the contributions of all markers are ranked, and markers with low values may be removed from the panel. The entire process can be repeated with the reduced number of markers until the desired panel size and performance are achieved.

Another embodiment of the invention measures multiple markers from a patient and combines the values into a single panel response. The panel response function could be determined by the method described above. The panel value would be compared to a cutoff value, providing an effective tool to aid in the diagnosis of disease states.

In a preferred embodiment, an objective function is a measure of overlap of panel responses of diseased patients and panel responses of non-diseased patients.

According to a preferred embodiment, the calculating of a value for an objective function includes generating a receiver operating characteristic (ROC) curve for the panel response. The ROC curve is indicative of a sensitivity of the panel response as a function of one minus a specificity of the panel response. ROC curves are well-known to those skilled in the art and are further described below.

In various aspects, multiple determination of the marker panels described herein can be made, and a temporal change in the markers can be used to rule in or out one or more diagnoses or prognoses. For example, one or more markers may be determined at an initial time, and again at a second time. In such embodiments, an increase in the marker from the initial time to the second time may be diagnostic of a particular disease, or indicate a particular prognosis. Likewise, a decrease in the marker from the initial time to the second time may be indicative of a particular disease, or of a particular prognosis.

In yet other embodiments, multiple determinations of marker panels can be made, and a temporal change in the marker can be used to monitor the efficacy appropriate therapies. In such an embodiment, one might expect to see a decrease or an increase in the marker(s) over time during the course of effective therapy.

In yet a further aspect, the invention relates to devices for analyzing the marker panels described herein. Such devices preferably contain a plurality of discrete, independently addressable locations, or “diagnostic zones,” each of which is related to a particular marker of interest. Following reaction of a sample with the devices, a signal is generated from the diagnostic zone(s), which may then be correlated to the presence or amount of the markers of interest. In preferred embodiments, one or more of the diagnostic zones comprise an antibody that binds for detection the particular marker to be detected at that particular addressable location.

The term “discrete” as used herein refers to areas of a surface that are non-contiguous. That is, two areas are discrete from one another if a border that is not part of either area completely surrounds each of the two areas.

The term “independently addressable” as used herein refers to discrete areas of a surface from which a specific signal may be obtained.

The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3^{rd} Edition, W. E. Paul, ed., Raven Press, N. Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

### BRIEF DESCRIPTION OF THE DRAWINGS

In the following, the invention will be explained in further detail with reference to the drawings, in which:

FIG. 1 is a chart illustrating an exemplary distribution of levels of a particular marker among a set of diseased patients and a set of non-diseased patients;

FIG. 2 is a chart illustrating an exemplary scatter distribution of levels of a particular marker among a set of diseased patients and a set of non-diseased patients;

FIG. 3 is an exemplary receiver operating characteristic (ROC) curve for the marker level distributions illustrated in FIG. 2;

FIG. 4 is illustrates the chart of FIG. 1 with the marker values being mapped to an indicator value;

FIG. 5 is a chart illustrating an exemplary scatter distribution of panel responses for the set of diseased patients and the set of non-diseased patients;

FIG. 6 illustrates a ROC curve for the panel response distributions of FIG. 5 with the knee of the ROC curve labeled;

FIG. 7 illustrates the progression of ROC curves through an optimization process;

FIG. 8 is a chart illustrating the relative contributions of each marker in a panel;

FIG. 9 shows the individual ROC curves and areas for each of 5 markers comprising the panel for FIGS. 6 and 16;

FIG. 10 shows the initial and final ROC curves for an optimization of 38 markers;

FIG. 11 shows the ranking and relative average contributions of 38 markers after 50 optimizations;

FIG. 12 shows the initial and final ROC curves for an optimization of 19 markers;

FIG. 13 shows the ranking and relative average contributions of 19 markers after 50 optimizations;

FIG. 14 shows the initial and final ROC curves for an optimization of 10 markers;

FIG. 15 shows the ranking and relative average contributions of 10 markers after 50 optimizations;

FIG. 16 shows the initial and final ROC curves for an optimization of 5 markers;

FIG. 17 shows the ranking and relative average contributions of 5 markers after 50 optimizations;

FIG. 18 shows the optimized ROC curves of 6, 3, and 2 measured and derived markers and 3 measured markers used to diagnose AMI; and

FIG. 19 shows the relative contributions of all 6 of the measured and derived markers for AMI.

### DETAILED DESCRIPTION OF THE INVENTION

In accordance with the present invention, there are provided methods and systems for the identification and use of a panel of markers for the diagnosis of one or more conditions or diseases, such as cardiovascular diseases and strokes, in a subject.

Method for Defining Panels of Markers

In practice, data may be obtained from a group of subjects. The subjects may be patients who have been tested for the presence or level of certain markers. Such markers are well known to those skilled in the art. A particular set of markers may be relevant to a particular condition or disease. The method is not dependent on the actual markers. The markers discussed in this document are included only for illustration and are not intended to limit the scope of the invention. Examples of such markers and panels of markers are described in pending U.S. patent application Ser. No. 10/139,086, entitled “DIAGNOSTIC MARKERS OF ACUTE CORONARY SYNDROMES AND METHODS OF USE THEREOF,” and U.S. patent application Ser. No. 10/225,082, entitled “DIAGNOSTIC MARKERS OF STROKE AND CEREBRAL INJURY AND METHODS OF USE THEREOF,” each of which is assigned to the assignee of the present application and is incorporated herein by reference. In accordance with the disclosed embodiments of the present invention, “markers” may also include factors such as a patient's history, sex, age and race, for example.

The group of subjects is divided into at least two sets. The first set includes subjects who have been confirmed as having a disease or, more generally, being in a first condition state. For example, this first set of patients may be those that have recently had a stroke. The confirmation of this condition state may be made through more rigorous and/or expensive testing. For purposes of this document, it will be assumed that this testing is able to confirm the condition state. Hereinafter, subjects in this first set will be referred to as “diseased”.

The second set of subjects are selected from those who do not fall within the first set. This set may include all remaining subjects, or only those subjects being in a second condition state. Subjects in this second set will hereinafter be referred to as “non-diseased”. Preferably, the first set and the second set each have an approximately equal number of subjects. The first and second sets of data are said to be a group of data. Multiple groups of data may be defined by repeating the steps above for different disease states, condition states, or any other selection criteria.

The data obtained from subjects in these sets includes levels of a plurality of markers. Preferably, data for the same set of markers is available for each patient. This set of markers may include all candidate markers, which may be suspected as being relevant to the detection of a particular disease or condition. Actual known relevance is not required. Embodiments of the methods and systems described herein may be used to determine which of the candidate markers are most relevant to the diagnosis of the disease or condition.

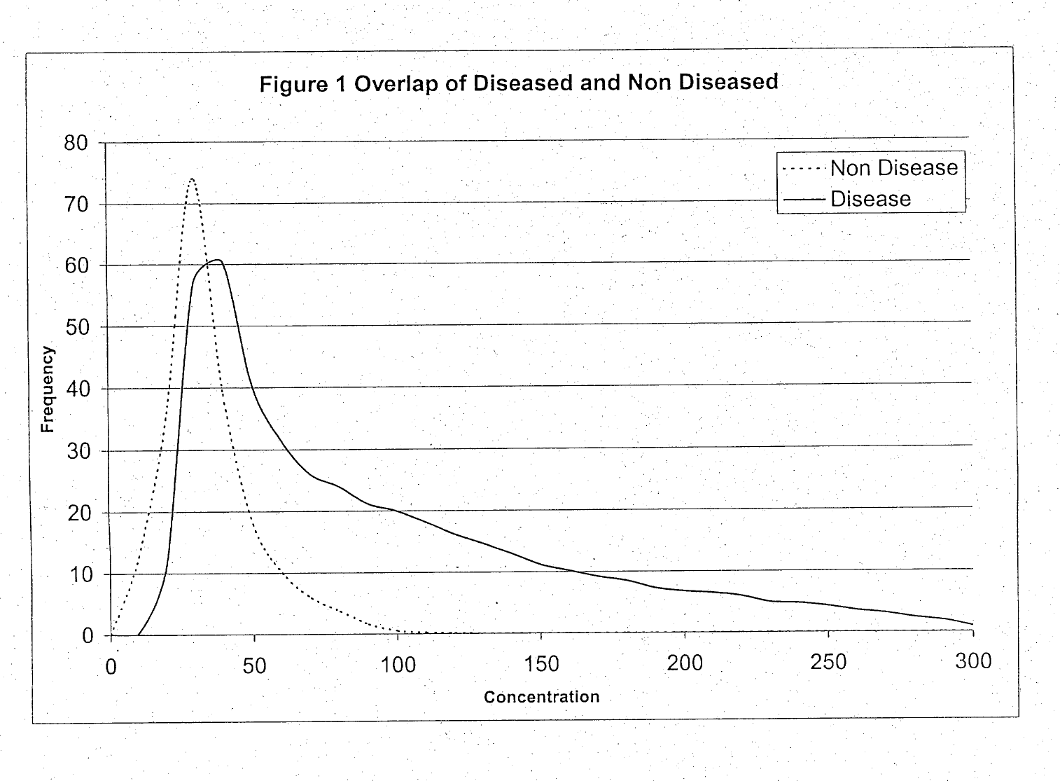

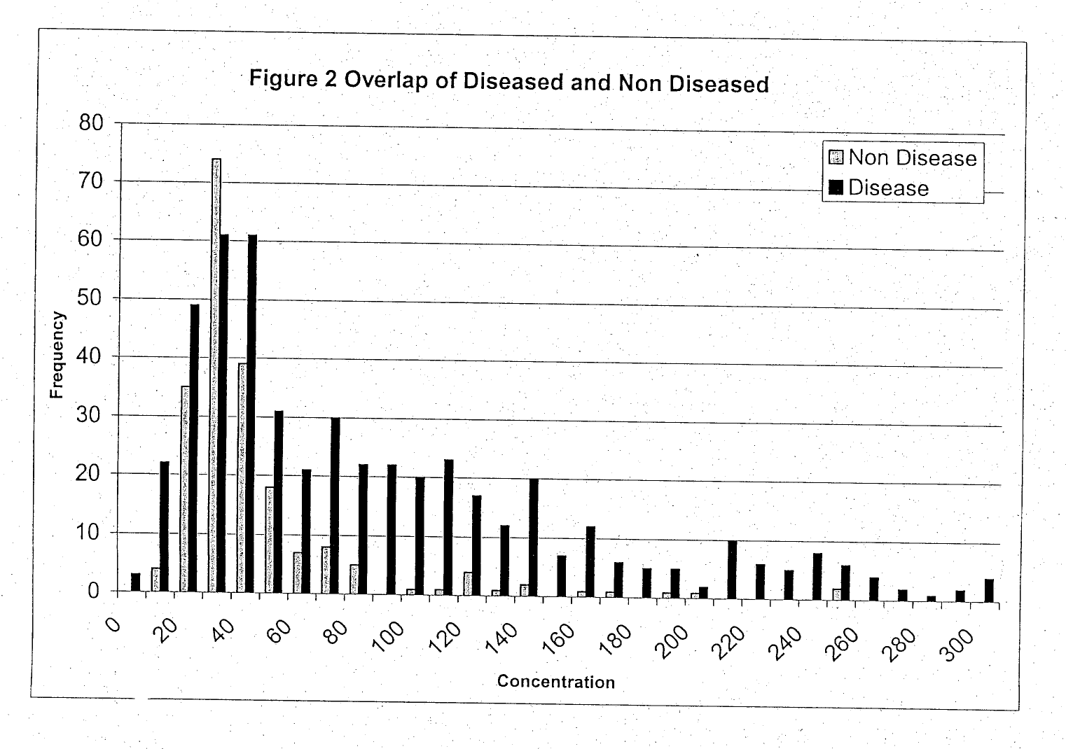

The levels of each marker in the two sets of subjects may be distributed across a broad range, as illustrated in FIG. 1. Further, although FIG. 1 illustrates a distribution for the marker levels of the two sets, data for the two sets may simply be available as data points for each patient, as illustrated in FIG. 2. No specific distribution fit is required.

As noted above and as illustrated clearly in FIGS. 1 and 2, a marker often is incapable of effectively identifying a patient as either diseased or non-diseased. For example, if a patient is measured as having a marker level that falls within the overlapping region, the results of the test may not be clinically relevant.

A cutoff may be used to distinguish between a positive and a negative test result for the detection of the disease or condition. Changing the cutoff trades off between the number of false positives and the number of false negatives resulting from the use of the single marker, or in the method described herein, the panel response.

The effectiveness of a test having such an overlap is often expressed using a ROC (Receiver Operating Characteristic) curve. Other measures, such as positive predictive value (PPV) and negative predictive value (NPV) may also be used as a measure of the effectiveness of the test. ROC curves are well known to those skilled in the art. For further details, see Zweig, MH. & Campbell, C. C., Clin Chem 39, 561-577 (1993) and Hendrson, A. R., Ann. Clin. Biochem 30, 521-539 (1993).

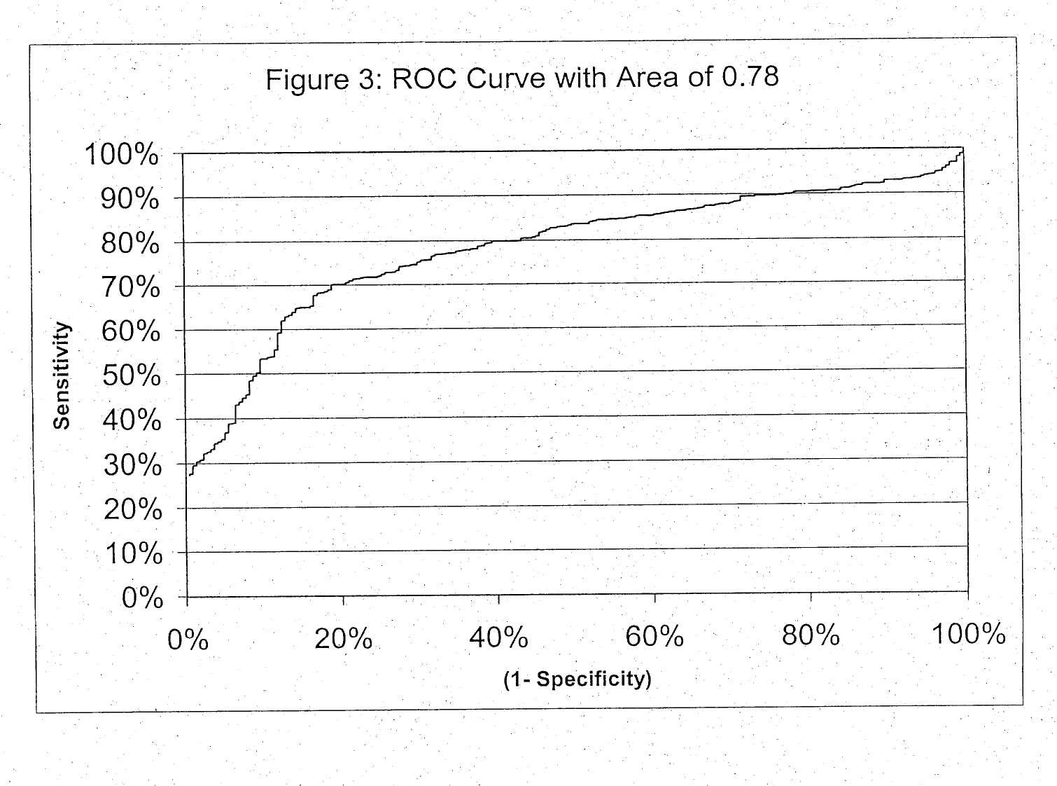

FIG. 3 illustrates an example of a ROC curve for the marker level distributions of FIG. 1. The horizontal axis of the curve represents (1-specificity), which increases with the rate of false positives. The vertical axis of the curve represents sensitivity, which increases with the rate of true positives. Thus, for a particular cutoff selected, the values of specificity and sensitivity may be determined. The area under the ROC curve is a measure of the utility of the measured marker level in the correct identification of one or more diseases or conditions. Thus, the area under the ROC curve can be used to determine the effectiveness of the test.

As discussed above, the measurement of the level of a single marker may have limited usefulness. The measurement of additional markers provides additional information, but the difficulty lies in properly combining the levels of two potentially unrelated measurements.

In the methods and systems according to embodiments of the present invention, data relating to levels of various markers for the sets of diseased and non-diseased patients may be used to develop a panel of markers to provide a useful panel response. The data may be provided in a database such as Microsoft Access, Oracle, other SQL databases or simply in a data file. The database or data file may contain, for example, a patient identifier such as a name or number, the levels of the various markers present, and whether the patient is diseased or non-diseased. Thus, a chart similar to FIG. 2 may be generated for each marker of interest. In practice, the generation of the chart is generally not required since the data may be directly accessible through the database or the data file.

In a preferred embodiment, one or more ‘derived markers’, which are a function of one or more measured markers, may be incorporated into the set of markers being studied. For example, derived markers may be related to the change in one or more measured marker values, or may be related to a ratio of two measured marker values. In many diseases there will be rapid change in marker value some time after an event. For example, following an acute myocardial infarction, (AMI), myoglobin may rise rapidly and peak about 3 hours from the event. It may then decay back to its nominal value. Looking for changes in markers can be powerful diagnostic tool. Thus, the change in myoglobin over a period of an hour, for example, may be used as a “marker” in the panel.

In practice diagnosis of a disease state from multiple markers can be confusing. Often the individual marker values may seem to contradict one another. In panels where the individual markers are not very effective, it is extremely difficult to understand their meaning. In a preferred embodiment, a function that combines the marker values into a scalar value that increases with increasing likelihood of disease is defined. In this manner, the information from multiple markers may be presented in a useable form. This defined function is referred to herein as the panel response (PR), and is a function of the marker values (M₀₋ₙ), written as PR=f (M₀₋ₙ). The panel response may be scaled such that all values are between 0 and 1. Because the effectiveness of the test may not depend on a scaling of the panel response, scaling may not influence the result of the method. However forcing the panel response to be a given scale may remove an unneeded redundancy, as panel response functions that differ only by a scaling factor may in fact represent the same solution. The panel response may also be a general function of several parameters including the marker levels and other factors including, for example, a patient's history, age, race and gender of the patient.

In a preferred embodiment, the panel response (PR) for each subject is expressed as:

$$PR = {\sum }_{Marker s} {I}_{i} ( {M}_{i} ) \cdot {W}_{i} ,$$

where i is the marker index, Wᵢ is the weighting coefficient for the marker i, Mᵢ is the marker value for marker i, I is an indicator function for marker i, and Σ is the summation over all candidate markers. The weighting factors scale the indicator functions and may allow for more important or specific markers to have a greater impact on the final panel response. The indicator function maps the marker value into a functional form appropriate to the marker's pathology. The indicator functions can be complex and should be chosen to match the marker. This will be illustrated in the embodiments described below. The indicator function may be a different functional form for each marker. In one example, the indicator function can map the marker value into a probability of the disease state. This mapping may not be a simple function of the marker value. In this example the said indicator from each marker can be summed to determine a relative index which is related to the probability of the patient being diseased. In a preferred embodiment the sum of all the weighting coefficients is constrained to a particular value, such as 1.0. In a preferred embodiment the indicator function is constrained to values between 0 and 1.

In a further preferred embodiment, both of the above constraints are satisfied, thus, the panel response is also constrained to a value between 0 and 1.

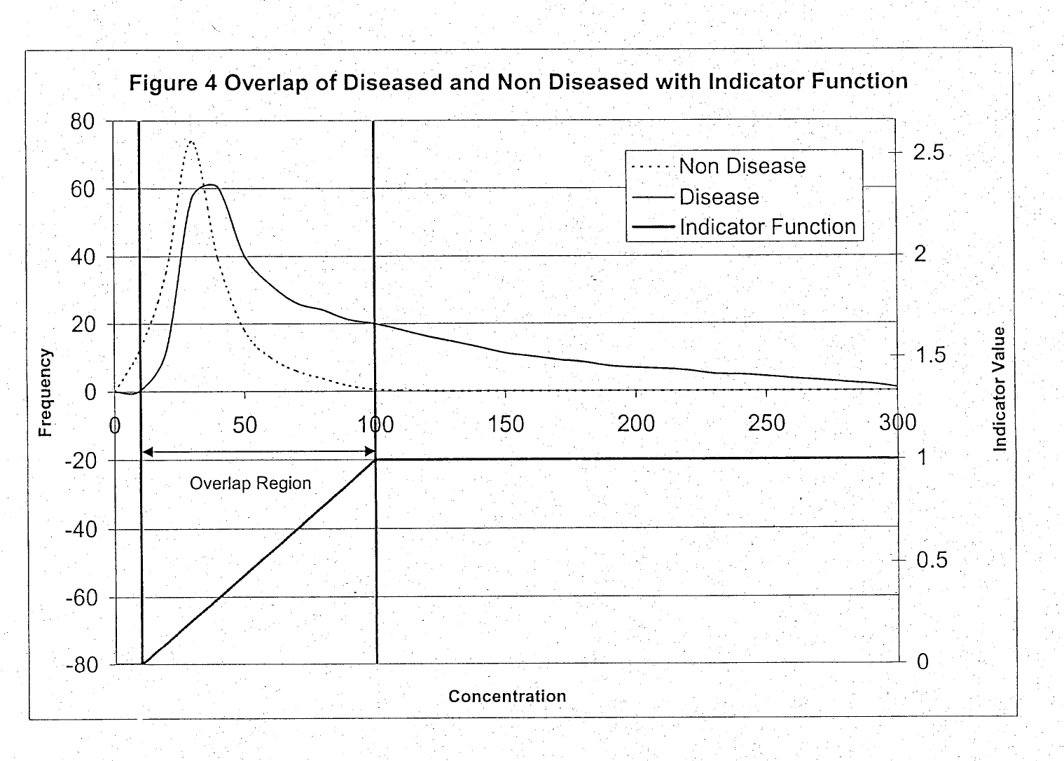

In many disease states such as stroke, nonspecific markers associated with that state are elevated. But above a certain threshold, higher values of the marker may not relate to a higher probability of disease state. Below a certain threshold, lower marker values may not relate to a lower probability of disease state. In this situation the indicator function may not increase linearly with the marker value. A preferred embodiment is an indicator function that is a function that has a high and monotonic rate of change between the thresholds, and a small rate of change elsewhere. Examples of this type of function are the ramp, step, or sigmoid functions. One may associate the lower threshold with the start of an overlap region (or cutoff region), and the upper threshold with the end of the overlap region, as shown in FIG. 4. Below the lower threshold the probability of disease is substantially 0, while above the upper threshold the probability of disease is 1. Note that in the case where the indicator function is a step function and the weighting value is 1 for each marker, then the panel response is simply the number of markers above the cutoff. This case is identical to the example used above where one is searching for the best panel with n of m markers above their cutoff. Allowing the indicator to vary continuously near the threshold enables the panel response to be sensitive to a marker just under the cutoff. This information is not lost as it is in the n of m marker example or the step function example, where the indicator value is not continuous. Another common approach of summing over M*W forces the linear relation with M. But as discussed above the most appropriate indicator function may not increase linearly with the marker value. In a further preferred embodiment the ramp function is used as an elevation indicator function. As illustrated in FIG. 4, the indicator values between the threshold regions may vary linearly from a value of zero at one end to a value of one at the other end. In other embodiments, non-linear variations of the indicator value may be used. The ramp function has the advantage of simplicity, and may be good approximation to other function in this class. With proper choices of parameters, the ramp function can be equivalent to the step function or can increase linearly with the marker value.

In some disease states, for example unstable angina, a specific marker such as the cardiac troponins (including isoforms of cardiac troponin, comprising troponin I and T and complexes of troponin I, T and C) may be elevated above the normal population, but further elevation indicates an acute condition, in this case a myocardial infarction. Unstable angina is an ischemic condition that leads to minor necrosis of cardiac tissue. During a myocardial infarction, there is major necrosis of cardiac tissue. Cardiac troponin, which is specific to cardiac necrosis, is elevated in both conditions, but the amount of elevation is related to the amount of necrosis. The best indicator function of cardiac troponin in diagnosing unstable angina may not be an elevation indicator function. In a preferred embodiment the indicator function may be a function that is peaked near the expected values of unstable angina, and decreases when the marker value is above or below the expected value. Examples of this type of function include a Gaussian, triangle, trapezoid, or square function. These functions tend to localize the marker value of interest around a specific value. Another example of use for such an indicator function is in cases where a pattern of markers values indicates a disease state. For example, a disease condition may be indicated when one or more markers are within a range of values. When desired, the use of this type of indicator may allow for recognition of patterns of marker values.

It is possible that one of the markers in the panel is specific to the disease or condition being diagnosed. An example of such a marker is cardiac specific cardiac troponin when used in the diagnosis of acute myocardial infarction. The role of TnI is described above. The panel response can be coded for markers that are specific, and the information may be used during the optimization of the panel response parameters. Typically the cutoff of such markers is known, so the cutoff values may not be included as a search parameter. When such a marker is present at above or below a certain threshold, the panel response may be set to return a “positive” test result, regardless of the levels of non-specific markers. When the threshold is not satisfied, however, the level of the specific marker may nevertheless be used as possible contributor to the objective function, along with the remaining markers on the panel.

In an example where the panel is being chosen based on n of m markers being elevated, the effectiveness of the panel is dependent on the choice of n. This extra dimensionality can be eliminated by using an objective function. The reduction of dimensionality may simplify the search process, and the objective function provides a scalar value that is optimized during the search process. The objective function should generally be indicative of the effectiveness of the panel, as may be expressed by, for example, overlap of the panel responses of the diseased set of subjects and the panel responses of the non-diseased set of subjects. In this manner, the objective function may be optimized to maximize the effectiveness of the panel by, for example, minimizing the overlap. In a preferred embodiment, the ROC curve representing the panel responses of the sets of subjects may be used to define the objective function. A ROC curve with a high value for the ROC curve area indicates a test with a good ability to discriminate between diseased and non-diseased. So, continuing with the n of m example above, there should exist a value of n which yields a clinically relevant test. The objective function is the scalar response that is maximized by the search algorithm. Other measures of effectiveness may include, for example, a positive predictive value (PPV) and a negative predictive value (NPV) of the panel. The PPV and NPV are well known to those skilled in the art. One skilled in the art will recognize there other measures of the effectiveness of the test. See The Immunoassay Handbook, Second Edition, David Wild, 2001 for measures of effectiveness. Many common measures of effectiveness require the selection of a cutoff value. These functions may still be used, and the cutoff value may also be included as a search parameter. In a preferred embodiment objective functions are chosen that do not require the selection of a cutoff value. The measure that is most appropriate for defining an effective test may vary.

In a preferred embodiment, the area under the ROC curve representing the panel responses of the sets of subjects may be used to define the objective function. Those skilled in the art will recognize that the area of the ROC curve is a measure of the effectiveness of the test. An area of 1 corresponds to a perfect test, and an area of 0.5 corresponds to a random test.

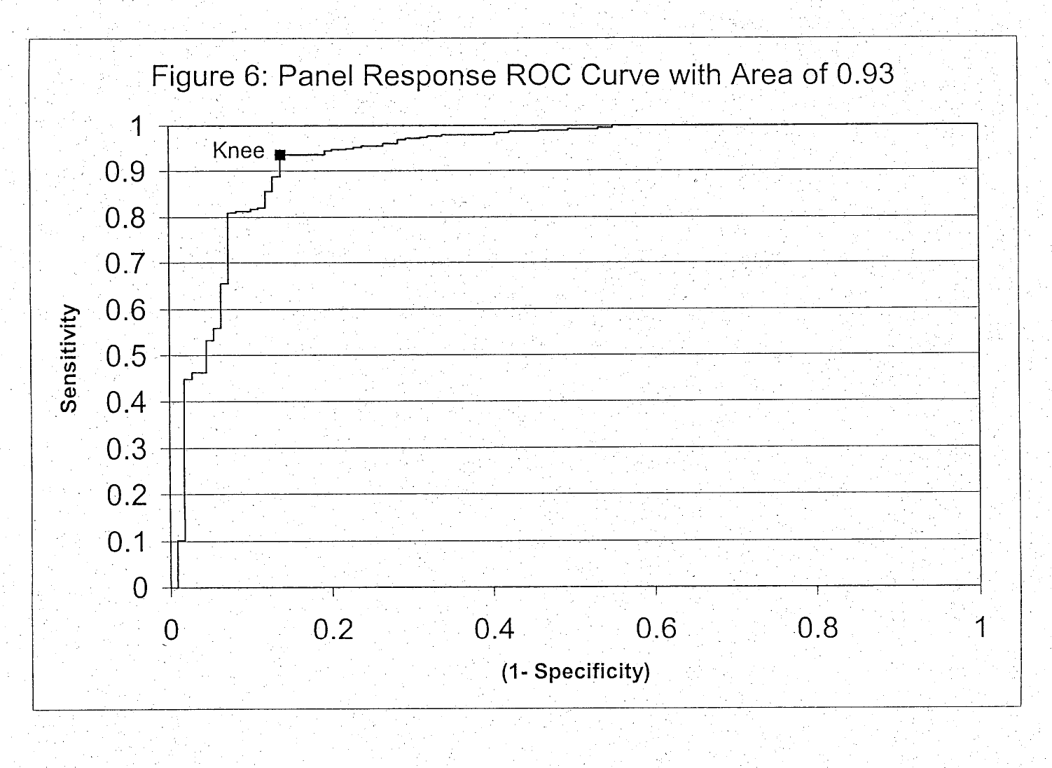

In another embodiment, the knee of the ROC curve is used for the objective function. The knee of the ROC curve is the point illustrated in FIG. 6, and the value is represented as the product of the specificity and sensitivity at the knee. In one embodiment the knee is found by maximizing the product of Specificity and Sensitivity. Higher knee values may indicate squarer ROC curves.

In another embodiment the objective function is the specificity at a prescribed sensitivity. If one requires that a test have only a certain sensitivity (ability to detected diseased patients) then maximizing the specificity, which may reduce the number of false positives, may improve the clinical effectiveness of the test.

In another embodiment the objective function is the sensitivity at a prescribed specificity. If one requires that a test have only a certain specificity (the number of false positives), then maximizing the sensitivity, which may increase the ability to detect diseased patients, may improve the clinical effectiveness of the test.

In a preferred embodiment, the objective function is the product of two or more characteristics of the ROC curve. An example of this is to use the product of the ROC curve area, knee, sensitivity at a prescribed specificity, and specificity at a prescribed sensitivity. Any one characteristic alone may not result in a desired solution. By using the product of two or more of these, a more desirable solution may be achieved.

Variations in the values of markers over some time interval within a patient may be a powerful tool in the diagnosis of disease states or condition or the progression of disease states or conditions. The panel response can be thought of as a new marker, where the panel response value is thought of as the marker value. Changes in the panel response value over some time interval within a patient may be a powerful tool in the diagnosis of disease states or conditions or the progression of disease states or conditions. The change in the panel response can be used as a derived marker. One can apply all of the ideas and methods discussed in this document to the case where a derived marker is the change in the panel response. Calling the change in the panel response a derived marker may be equivalent to defining a new panel response that is the change in the panel response over some time interval. The new panel response function is a function of the marker values at two time points. All methods and ideas discussed in this document can apply to the new panel response.

Searching for the best panel can be accomplished by trying all the different combinations of parameters of the panel response function. But with panels of 40 markers, and just one degree of freedom per marker, taking 10% steps in the parameter values will require 10⁴⁰ iterations. The age of the universe is estimated to be about 20 billion years or about 6.3×10¹⁷ seconds. Clearly this approach is not practical, and the problem requires the use of a search engine. Optimization algorithms are well-known to those skilled in the art and include several commonly available minimizing or maximizing functions including the Simplex method and other constrained optimization techniques. It is understood by those skilled in the art that some minimization functions are better than others at searching for global minimums, rather than local minimums. Many of these exist, and detailed descriptions can be found in the literature. For more information on minimization and maximization functions, reference may be made to Numerical Recipes in C, The Art of Scientific Computing, Second Edition, W. Press, et al., Cambridge University Press, 1992, which is hereby incorporated by reference. The panel response and the objective function have helped enable the use of search routines. The objective function value is the response that the search routine will maximize, and the parameters of the panel response function form the n-dimensional space to be searched. While the objective function does not need to be continuos, i.e. it may have discrete values, panel response functions that are continuos may reduce the granularity of the objective function. This may help the algorithm find better solutions. While many search routines will in fact look for minima, the problem may be inverted by minimizing (−1)*Objective Function.

In a preferred embodiment the search engine uses the Downhill Simplex Method in Multidimensions. This method is described in Numerical Recipes in C, The Art of Scientific Computing, Second Edition, W. Press, et al., Cambridge University Press, 1992. The simplex has n+1 vertices, where n is the number of dimensions or degrees of freedom. The routine ‘walks’ the simplex along the n dimensional surface, moving one vertex at a time. The scale of the simplex can change so it can both quickly walk in downhill directions and crawl through tight crevices. The routine may not find a global minimum because it can become trapped in a local minimum. The simplex will search all real space. The parameters of the panel response are often valid only within some range, defining the bounds of the system. The simplex must be constrained to only search in this space, and there must be no degeneracy introduced when approaching such a constraint. One skilled in the art will recognize that there are many ways to address this constraint. An effective method is to assess a penalty when a vertex moves out of bounds. This penalty creates steep canyon wall around the bounds of the system, effectively constraining the simplex within the bounds of the system.

A well-known limitation of search engines is their tendency to find only a local minimum, typically not the global minimum. Several techniques are known to improve the ability to seek out the global minimum. In a preferred embodiment, the technique of simulated annealing is used. This method is also described in Numerical Recipes in C. The Art of Scientific Computing, Second Edition, W. Press, et al., Cambridge University Press, 1992. Simulated annealing adds a random error to each decision of the search engine. This random error gives the search engine the ability to move out of a shallow local minimum, so it can seek out a deeper one. The random error is systematically reduced until a minimum is found. The random error is similar to the effect of temperature in annealing processes. The scale of the random error is said to be the temperature. The annealing process may improve the chances of finding a global, rather than local, minimum. The annealing process may result in a more stable solution since the random variation may move the simplex out of a narrow, unstable region. The optimization process may be terminated when the difference in the objective function between two consecutive iterations is below a predetermined threshold, thereby indicating that the optimization algorithm has reached a region of a local minimum. The number of iterations may also be limited in the optimization process.

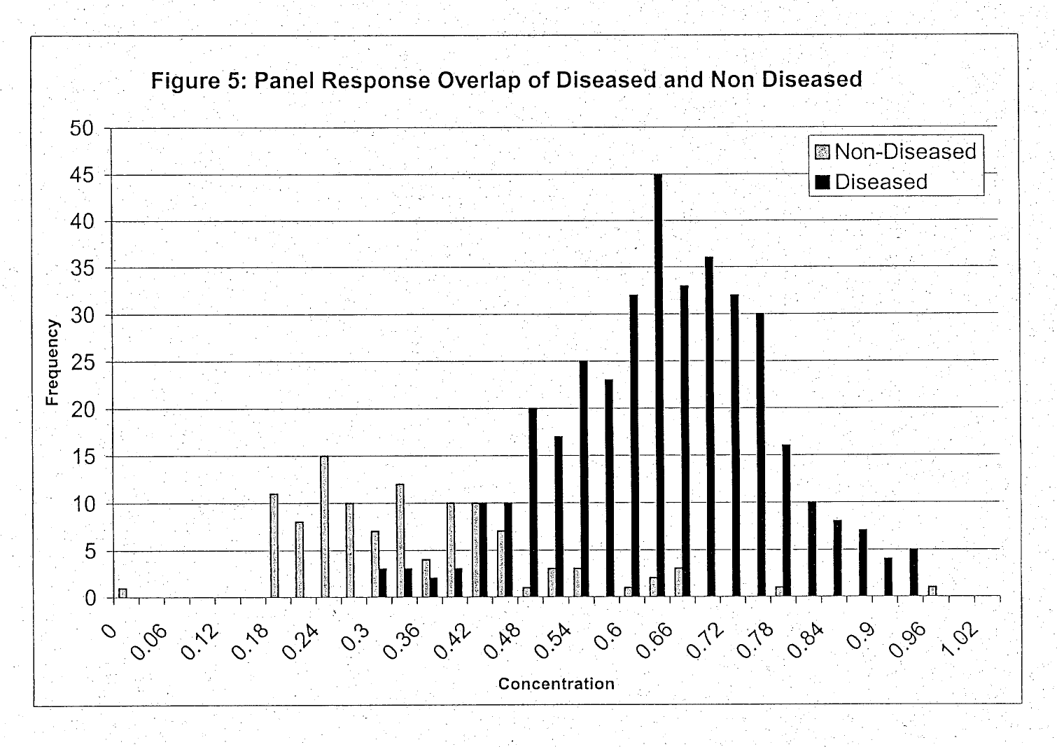

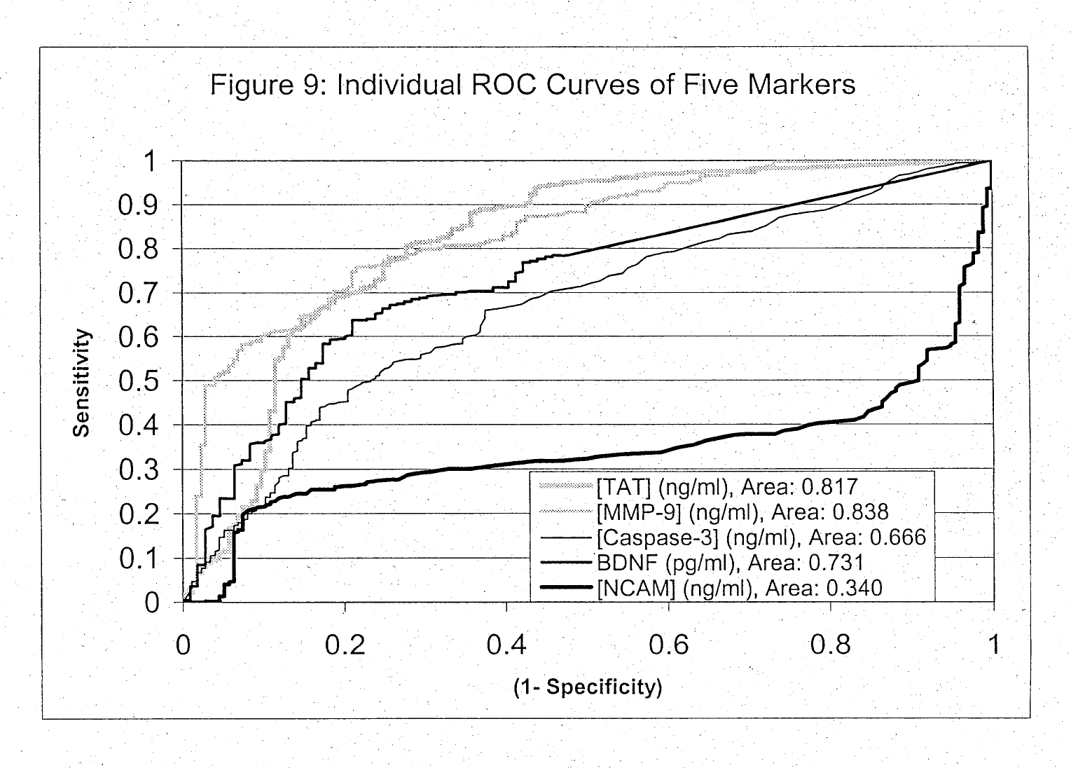

The selection of the initial conditions, for example the initial simplex value, may affect the optimization process. So, generally good selections of the initial parameters are sought. In the example of a search using a simplex, all vertices of the simplex must be initialized. If only one good vertex is defined, the other vertices can be assigned by applying a random deviation to each parameter. The scale of this random deviation sets the scale of the initial simplex. For example when elevation indicator functions are used, the location of the cutoff region may initially be selected at any point. But, selection near a suspected optimal location may facilitate faster convergence of the optimizer. In a preferred method, the cutoff region is initially centered about the center of the overlap region of the sets of patients. In one embodiment, the cutoff region may simply be a cutoff point. In other embodiments, the cutoff region may have a length of greater than zero. In this regard, the cutoff region may be defined by a center value and a magnitude of length. In practice, the initial selection of the limits of the cutoff region may be determined according to a pre-selected percentile of each set of subjects. For example, a point above which a pre-selected percentile of diseased patients are measured may be used as the right (upper) end of the cutoff region. In another embodiment the weighting factors may initially be all set to one. In a preferred embodiment, the initial weighting coefficient for each marker may be associated with the effectiveness of that marker by itself. For example, a ROC curve may be generated for the single marker, and the area under the ROC curve may be used as the initial weighting coefficient for that marker. This gives more weight to markers with better univariate utility. Having selected parameters for the panel response function, the panel responses for each subject in each set of subjects, and the distribution of the panel responses for each set may now be analyzed. FIG. 9 shows the ROC curves and area of several markers that have a poor diagnostic utility. The markers data are used to generate FIG. 5. When the poor markers are combined and the panel response determined, the results show that the panel now has enhanced utility. FIG. 5 illustrates an exemplary distribution of the panel responses for diseased and non-diseased subjects. Based on these distributions, a ROC curve may be generated, as illustrated in FIG. 6. The ROC curve illustrated in FIG. 6 reflects optimized values for the weighting coefficients and the thresholds for a ramp indicator function.

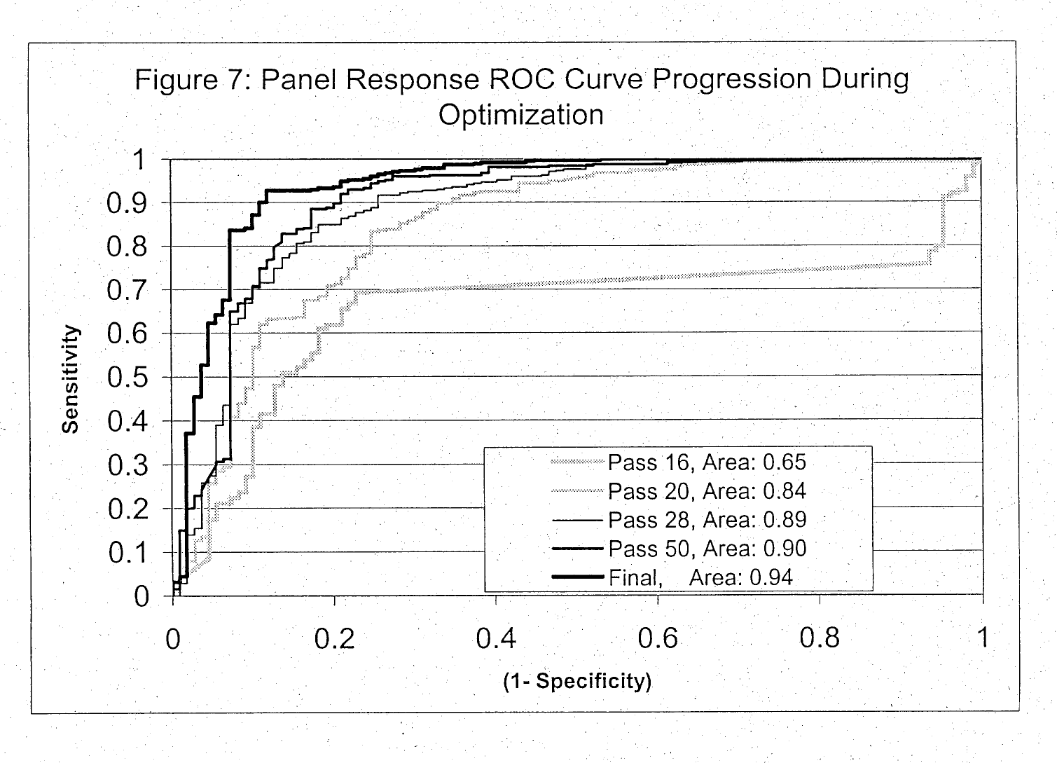

FIG. 7 illustrates an exemplary progression of a ROC curve through a plurality of iterations of an optimization process in which the objective function is defined as the area under the ROC curve. As illustrated in FIG. 7, as the number of iterations increases, the area under the curve may progressively increases. Thus, the optimization process may provide a panel response function for the markers. In this example, the indicator function is a ramp function. The optimization routine found values of the weighting coefficients and high and low threshold values which are represented as a cutoff value and linear range. Table 1 illustrates a panel of 38 candidate markers with weighting coefficients and cutoff regions resulting from the optimization process. The 38 markers are listed generically as Analyte 1 through Analyte 38. The sense of each marker, as described above, is also indicated in Table 1, with “Incr” representing a positive sense and “Decr” representing a negative sense. The cutoff location indicated in Table 1 refers to the marker level value around which the cutoff region is centered, while the length of the cutoff indicates the range of marker level values covered by the cutoff region. In this manner, any number of markers may be used to develop a highly effective panel response function that can be used for the diagnosis of a disease or condition.

The result of any given search is likely not to be the global minimum. It may be any local minimum that the search engine settled in. In a product to be used for clinic diagnosis, it is preferable to find a very stable solution. Inaccuracy associated with the measurement of the marker values should not significantly influence the effectiveness of the test. Also, the defining data may not be inclusive of all patients; it may be only a small sample, and the remaining population may deviate from the defining population. The desired characteristics of the minimum may include a wide width and shallow side walls. In a three-dimensional analogy, we would prefer a minimum like a crater as opposed to a mine shaft. One method to seek out these types of solutions is to search multiple times. If a statistically significant number of optimizations is performed, then the better solutions will be the largest group of similar results. This is because, using the example above, it is more likely to find the crater than the mine shaft.

As discussed above, not every minimum found may be desirable to use. Generally stable parameters are desired, meaning that variations in the marker values or parameters do not adversely impact the effectiveness of the test. There are several examples of methods that may quantify the quality of a set of parameters. A first example is to vary the marker values by some random percentage. By doing this one can simulate all the variations expected due to assay imprecision, biological variations, and any other source of uncertainty. For example, variations in marker values may relate to the relative imprecision of the test that was used to generate the data. One skilled in the are will recognize that there are limits to the analytical precision of a test. For example, in the immunoassay art, it is common to encounter 5-20% coefficients of variations of the tests. Therefore, when considering the imprecision of the testing methodology, the parameters remain stable relative to the imprecision of the methodology. The randomized data set can be reanalyzed to generate the new panel response ROC curve and objective function value. An acceptable deterioration may indicate the parameters give a solution that is stable to variations in marker values and may also verify that the solution does not simply fit the noise in the data. A second example would be to vary one or more of the parameters in the panel definition some amount. The change in the objective function value may be a measure of the quality of the solution. Each parameter could be varied independently to determine the stability of each parameter. The width and depth of the minimum may also be measures of the stability of the solution. In a third example, a seed simplex is generated with a given length scale about the known minimum. The length scale of the seed simplex can be systematically increased until re-optimizations lead to a different minimum, i.e. the solution is no longer recovered. The length scale, which results in finding new minimums, may be related to the width of the minimum. In a fourth example, using the final simplex of the optimization, the temperature can be systematically increased until re-optimizations lead to a different minimum, i.e. the solution is no longer recovered. The temperature, which results in finding new minimums, may be a measure of the depth of the solution. In a fifth example, most common solutions from the multitude of optimizations, may represent the most stable solution. The common solutions can be grouped based on their similarity. Correlation techniques and clustering techniques can be used to group the solutions, and are well known to one skilled in the art. From the teaching above, it is now clear that other approaches exist for quantifying the quality of a set of parameters, and the examples above are not intended to limit the invention.

The use of the term “non-diseased” does not mean that the particular subject is disease-free, only that the subject is free from the one or more diseases or conditions being evaluated. In practice, a pre-filtering of subjects may be performed on the basis of any particular characteristic of the subjects, including the existence of other diseases. For example, the method and systems described may be applied to first divide a group of subjects into “diseased” and “non-diseased” for Disease A, and then divide the group into “diseased” and “non-diseased” for Disease B. A panel of markers for each disease may then be determined. In another embodiment, the same panel of markers may be used for both diseases with a different set of parameters, such as weighting coefficients, for each disease. In another embodiment subjects with disease A can be defined as non-diseased, and subjects with disease B can be defined as diseased. In this embodiment the described techniques can be employed to determine a panel that differentiates between diseases A and B.

The search routine will optimize the objective function or functions selected on the specified data set. But, often times it is important to constrain or optimize a second group of data simultaneously. This is accomplished by pre-filtering the source data to get the two or more groups of data of interest. Different objective functions can be selected for each group of data, and the search engine can find the minimum of the product of objective functions. The objective function of one of the groups of data can also be constrained to be at least some value. When the objective function is greater than or equal to this constraint, the value returned to the search engine is the constraint value. When the objective function is below the constraint value the objective function value is returned. The search routine will look for solutions that satisfy the constraint condition, but the best solution may fall outside the constraint condition. The iterations of the optimization algorithm generally vary the independent parameters to satisfy the constraints while maximizing the objective function. An example of this usage is stroke data that contains norm health donors and stroke mimics. We would like to find a panel response function that will distinguish stroke from stoke mimics, but that will also have a low false positive rate for normal healthy donors (NHD). Since the number in each sample set is not equal, simply combining the data and analyzing will not give a satisfactory result. Results will be skewed to the data set with larger n, in our case NHD. However, if the objective functions of the two groups of data are individually calculated and combined, then the groups of data are given equal weight. In another example we want to ensure that patients presenting soon after the onset of symptoms will be properly diagnosed, but we still want to ensure that patients presenting at longer times are also properly diagnosed. Again, the population numbers will be different. So, to give equal weighting, they need to be simultaneously analyzed as two groups of data. Other constraints may include limitations on one group of samples while optimizing for an objective function for a second group. For example, a panel may be optimized for one disease while the same panel may be constrained to provide at least an acceptable minimum value for the area under a ROC curve for a second disease.

Within the teachings of this document we have used for simplicity markers that are elevated in patients with the disease or positive sense markers. However this is not always the case, and often, particularly with poor univariate markers, it is not clear from univariate analysis whether the marker when used in conjunction with the other markers in the panel, is best utilized in a positive or negative sense. If the sense of a marker is inverted, then it is straightforward to invert the indicator function for that marker. If the sense is not known, then the search engine may include this as a degree of freedom. For example, in one embodiment, the sense may be a truly separate independent variable, which may be flipped between positive and negative by the optimization process. For optimal performance, the sense should map smoothly from improper to proper, and there should be pressure that allows the search engine to move toward the proper sense. In a preferred embodiment the sense is switched by allowing the weighting coefficient of the analyte to go negative. If the wrong sense is selected, the weighting coefficient will be driven towards zero since inclusion of the marker in the panel response negatively impacts the objective function. The search engine will be able to drive the weighting coefficient across zero to the proper sense.

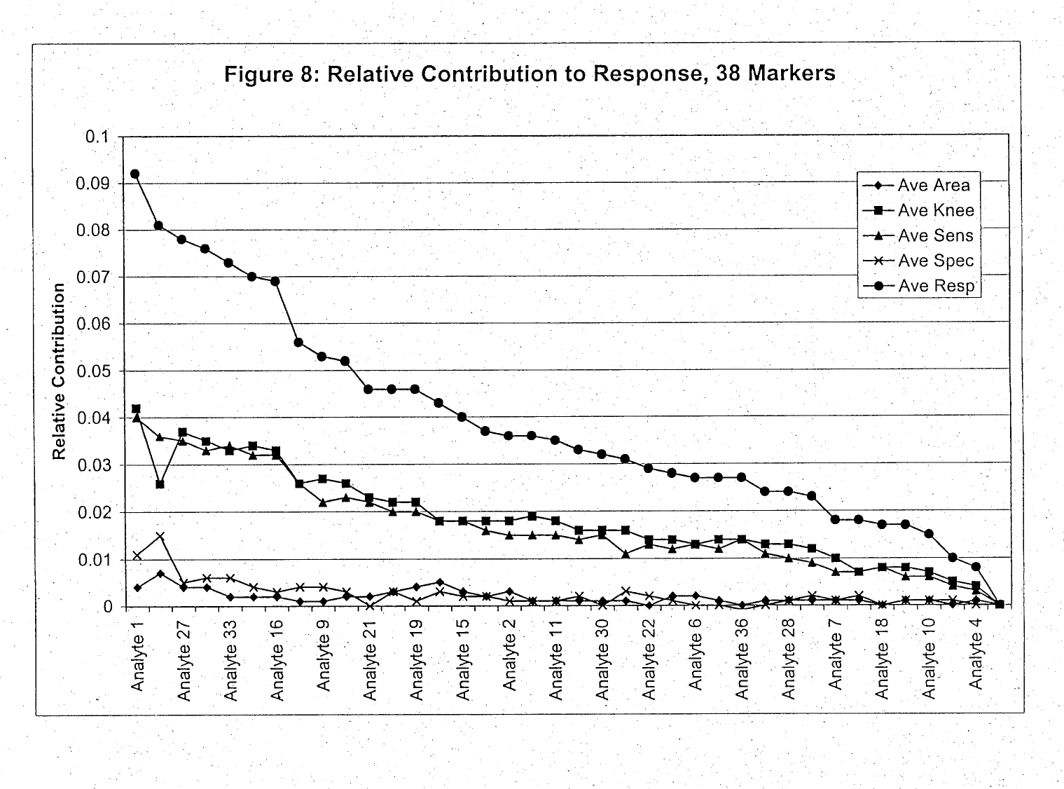

In order to determine the best panel, which for practical reasons may often mean 10 or less markers, one must find a way to systematically remove markers that do not significantly contribute to the overall result. This is accomplished by calculating the contribution from each marker. A method to accomplish this is to remove an analyte from the panel, and recalculate the objective function. The change in the objective function is related to the contribution of the marker. This method for identifying the relative importance of each marker is illustrated in FIG. 8. The resulting changes in the objective function are noted for each marker and plotted, as shown in FIG. 8. FIG. 8 illustrates the effect each marker has on the various features of the ROC curve corresponding to the panel responses for the two sets of subjects. The various ROC-curve features noted in FIG. 8 include the area under the ROC curve, the location of the knee of the ROC curve, the sensitivity at a predetermined specificity, and the specificity at a predetermined sensitivity. The markers may then be arranged in order of decreasing contribution, as illustrated in FIG. 8. The vertical axis in FIG. 8 indicates the relative change in the values of the various ROC-curve features. In embodiments where a weighting coefficient is applied to each analyte, the weight for the analyte can be set to zero to remove the analyte from the panel. In embodiments where a weighting coefficient is applied to each analyte, one can not simply use the weights as the contribution. An example of why this does not give the proper result is the case where a marker has zero impact on the test. In this case, the weight it is given by the search program can be any value, so it is possible that its weight will be the highest.

In order to develop lower-cost panels, which require the measurement of fewer marker levels, certain markers may be eliminated from the panel. In this regard, the effective contribution of each marker in the panel may be determined to identify the relative importance of the markers. Once the relative contributions are calculated then one can rank them from largest to lowest. The markers with the largest changes in objective function may be the ones with most contribution. The ones with the least change in objective function may be the ones with the least contribution. If two markers are perfectly correlated, then the combined contribution from both may be equivalent to the contribution of just one if the second one is removed. The partitioning of the contributions is not necessarily equal. So an important marker may not have a high contribution. This problem can be avoided by first looking at the correlation between markers, or by removing only one marker or more with the lowest contribution.

From the discussion above, it is noted that it may not be prudent to just select the top 3 markers from a panel of 40. Depending on the number of target markers being searched and the size of the target panel, one may want to eliminate only the marker with the lowest contribution or the lowest markers, and repeat the process until the target panel size is reached. With properly defined panel responses, markers of no importance may not adversely impact the objective function. This is because a) the search routine may chose parameters such that the marker is not used, and b) in general a random marker will not change the objective function. So, starting with a large panel and reducing it to the desired size will lead to the optimum panel. But the objective function may degrade as markers are eliminated. One may have to trade off panel effectiveness with the number of markers. For example, in order to obtain a panel of ten markers, the ten highest-rated markers, i.e. those on the left side in FIG. 8, may be selected. For example, Analytes 38, 1, 16, 33, 27, 12 and 8 may be selected in a final panel of markers. In a preferred embodiment, only a few of the markers on the right side may be eliminated, and the remaining markers in the panel may be optimized. For example, Analytes 31, 24, 25, 4 and 10 may be eliminated in a first round, and the optimization and ranking procedures may be repeated with the remaining 33 markers. This results in a chart similar to that shown in FIG. 8, but with fewer markers. This process may be repeated until a desired number of markers remains in the panel.

It is possible that one of the markers in the panel is specific to the disease or condition being diagnosed. An example of such a marker is cardiac specific TnI when used in the diagnosis of acute myocardial infarction. The role of TnI is described above. The panel response can be coded for markers that are specific, and the information is used during the optimization of the panel response parameters. Typically the cutoff of such markers is known, so the cutoff values may not be included as a search parameter. When such a marker is present at above or below a certain threshold, the panel response may be set to return a “positive” test result, regardless of the levels of non-specific markers. When the threshold is not satisfied, however, the level of the specific marker may nevertheless be used as possible contributor to the objective function, along with the remaining markers on the panel.

In a preferred embodiment the panel will include markers derived from the rate of change of markers measured by the panel. In a further preferred embodiment the panel will have two panel response functions, one that utilizes the derived markers when present, and when not present one that does not utilize the derived markers. The two panel response functions may use different parameters. These parameters may be obtained by optimizing the data with and without utilizing the derived marker or markers. For example, a patient may be measured when first arriving at the hospital for a particular set of markers. Since there is only one sample time for the patient a panel response function which does not include marker changes is used. The patient would be diagnosed as diseased or non-diseased based on the results of the test. The same patient may be measured again an hour later. Now there are two points, and so a second panel response function which utilizes marker changes is used. The use of this response function is important when a marker or panel of markers of disease indicates non-disease, but the change (usually increase) in the value of one or more markers represents the start of disease.

It is possible for a panel of markers to contain enough information to diagnose a multitude of conditions. In the simplest case, the markers used in the diagnosis of condition A are different from the markers used in the diagnosis of condition B. In a preferred embodiment, the markers used in the diagnosis of condition A contains at least one of the markers used in the diagnosis of condition B. In a future preferred embodiment there is a high degree of overlap in the markers used to diagnose a multitude of conditions.

The method described above may be implemented in a variety of manners. In a preferred embodiment, the method is implemented as a program product, such as a software package. The program product may be implemented on a computer, such as a personal computer, a mainframe or a handheld device. It will be apparent to those skilled in the art that the program product may be implemented on a device in any number of ways including software, firmware, etc. In one embodiment, the program product is implemented on a meter which may be capable of directly measuring levels of one or more markers. For example, the program product may be implemented on a fluorometer or a reflectometer. Such devices are well known to those skilled in the art.

In a most preferred mode, patient types, disease types, and time frames are selected to provide two data sets, diseased and non-diseased, which have the characteristics to be evaluated. Multiple groups of data can be selected, each set consisting of a set of diseased and as a set of non-diseased samples. The values for any derived marker values of interest are calculated for each record in the selected groups of data. This may include calculating the change in marker value from the initial value. Based on the disease and marker pathology, a functional type for the indicator function is chosen for each marker to be included in the panel. The teachings in this document should enable one skilled in the art of the disease and marker to make the appropriate choice. Once the indicator functions have been defined, then the initial parameters are chosen from the univariate marker analysis. These initial parameters define one vertex of the initial simplex. The number of vertexes constituting the simplex is the number of search parameters in the panel response plus one. Each remaining vertex is populated by varying each parameter by a random amount. The scale of this random amount can be fixed to be a percentage of the parameter value. This spreads the simplex out around the initial point, and gives the simplex a size scale. The objective function for each group of data is defined by selecting any combination of the ROC curve area, the ROC knee, the ROC sensitivity, and the ROC specificity, but typically all four are selected. The objective function for each group of data can be chosen to be optimized or to maintain a minimum target value. Thus the optimization of one group of data can be constrained such that a second group of data has at least a minimum objective function value. The parameters are then optimized to maximize the chosen objective function utilizing the downhill simplex method with simulated annealing. At the end of the optimization the relative contribution for each marker is calculated by setting the weight of that marker to zero and recalculating the panel ROC curve. When the analyte is so removed from the panel response, the new ROC curve is calculated with the identical data and no other parameters in the panel response are changed. The process of optimizing and calculating marker contributions is repeated n (˜100) times. After n optimizations, the average contribution of each marker over the n optimizations is calculated, and the markers are ranked based on its average contribution. The poorest markers, typically the poorest half or less, are removed from the panel and the entire process is repeated as many times as required to reduce the panel to the desired size.

Using optimal analytes and parameters for the panel response function found via the search method described above, the ROC curve of the panel response from clinical data is calculated. Based upon the panel response ROC curve an appropriate cutoff is chosen. The choice may be influenced by factors such as clinical factors, treatment methods, and cost considerations, which one skilled in the art will recognize. The panel response is calculated from the measured marker values of the patient for whom it is desired to determine the presence or absence of the target disease. Using the chosen cutoff, assign a diagnosis for the patient.

Using optimal analytes and parameters for the panel response function found via the search method described above, for panel response functions which include and exclude markers derived from the change in a measured marker, the ROC curve of the panel response from clinical data is calculated. Based upon the panel response ROC curves appropriate cutoffs are chosen for each. The choice may be influenced by factors such as clinical factors, treatment methods, and cost considerations, which one skilled in the art will recognize. Upon measurement of the initial sample, the panel response is calculated from the measured marker values of the patient for whom it is desired to determine the presence or absence of the target disease. Using the chosen cutoff, assign a diagnosis for the patient. A second or more measurement may be required to further clarify the diagnosis. At the appropriate time interval, draw more sample from the patient and measure the marker values. Using the panel response function that includes derived markers, calculate the panel response value and determine a diagnosis by comparing the panel response value to the chosen cutoff value. The panel response of the first measurement can also be compared to panel responses determined from subsequent measurements. One skilled in the art will recognize that serial blood draws can yield critical information of the presence and progression of diseases, particularly acute diseases. If more measurements are required for proper patient treatment, continue taking samples at the desired intervals.

### EXAMPLES

### Example 1

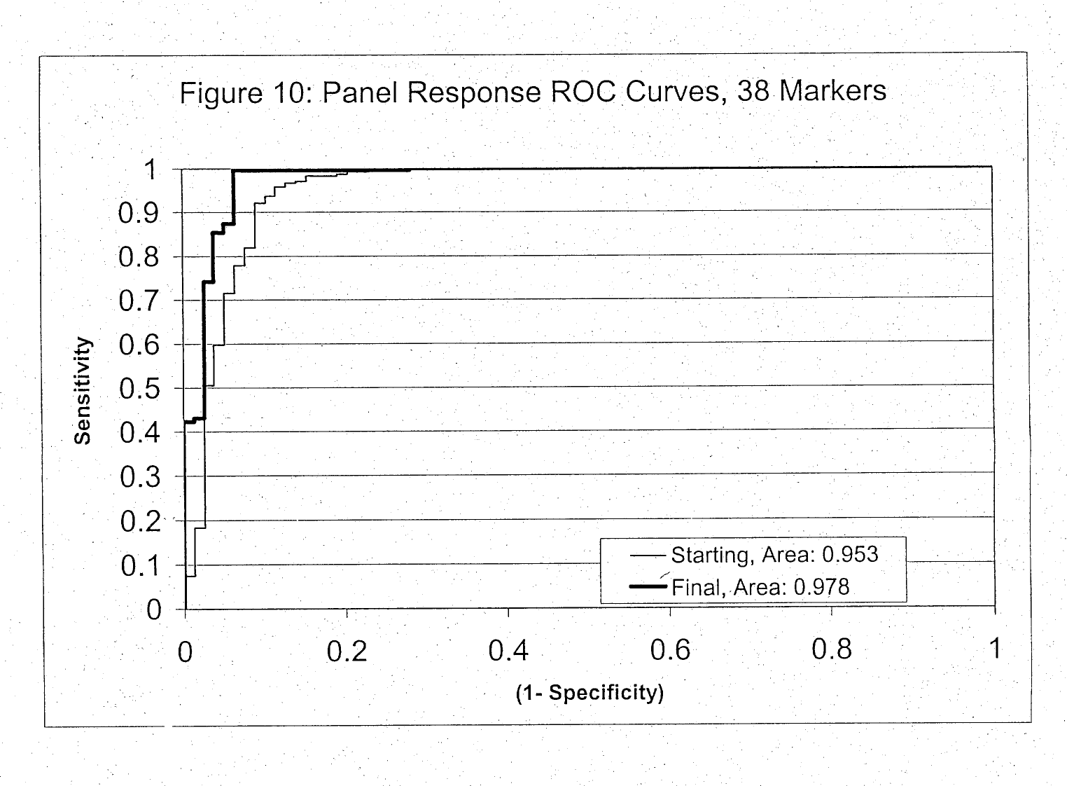

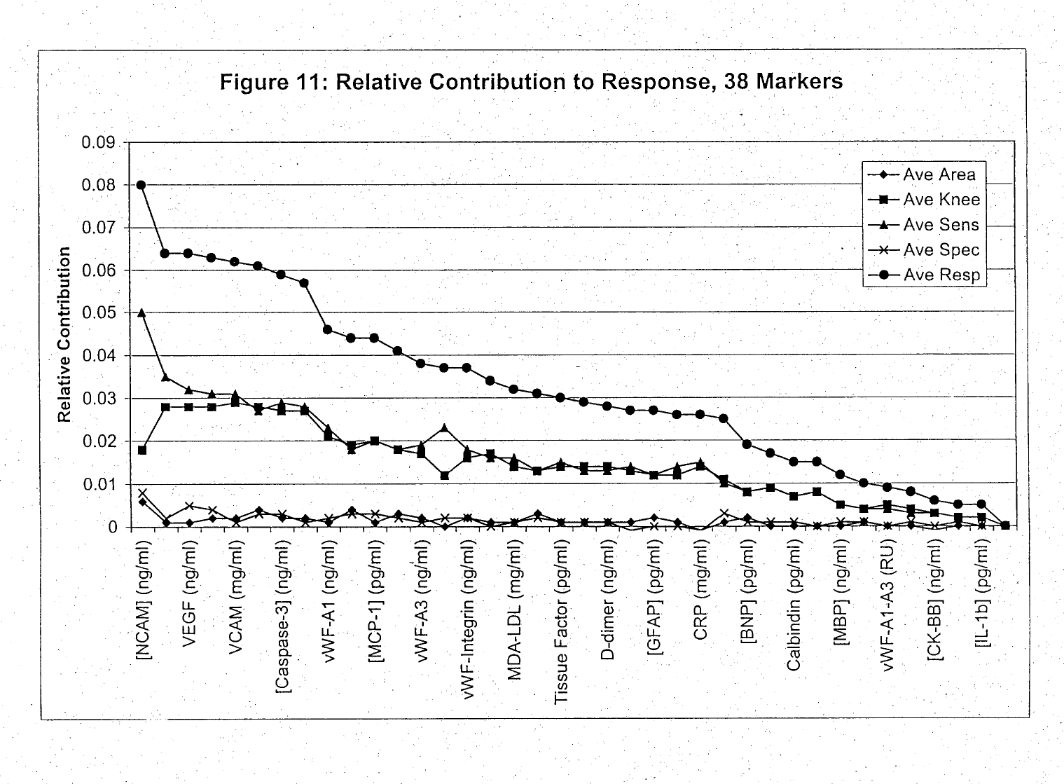

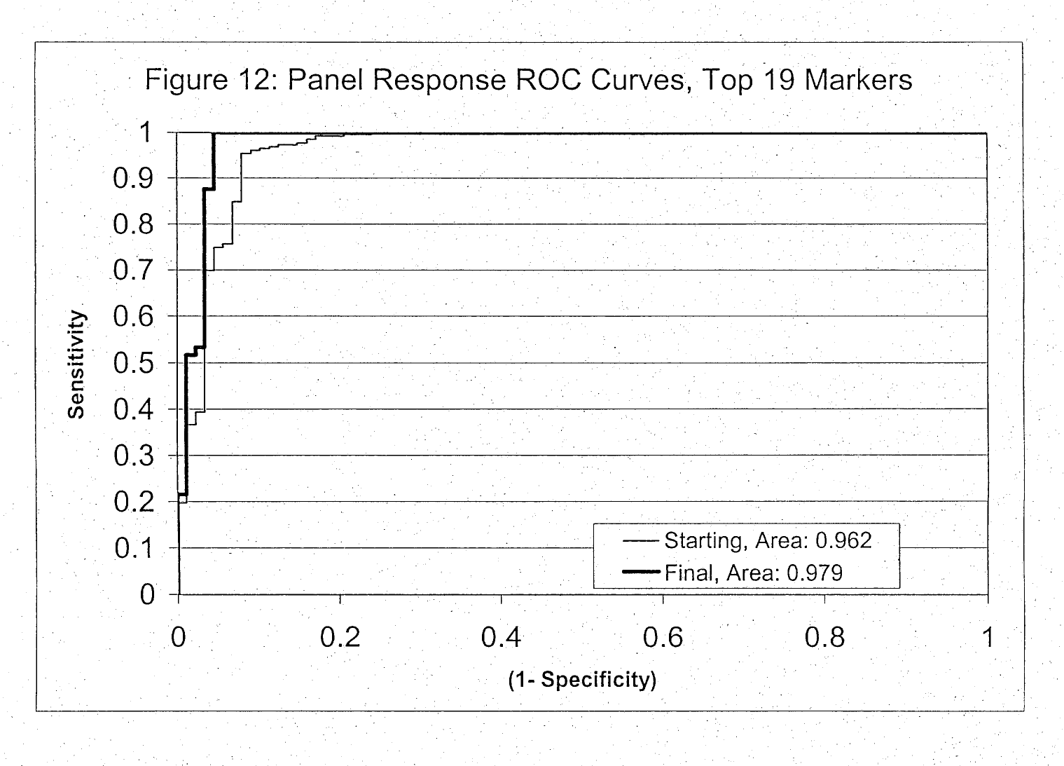

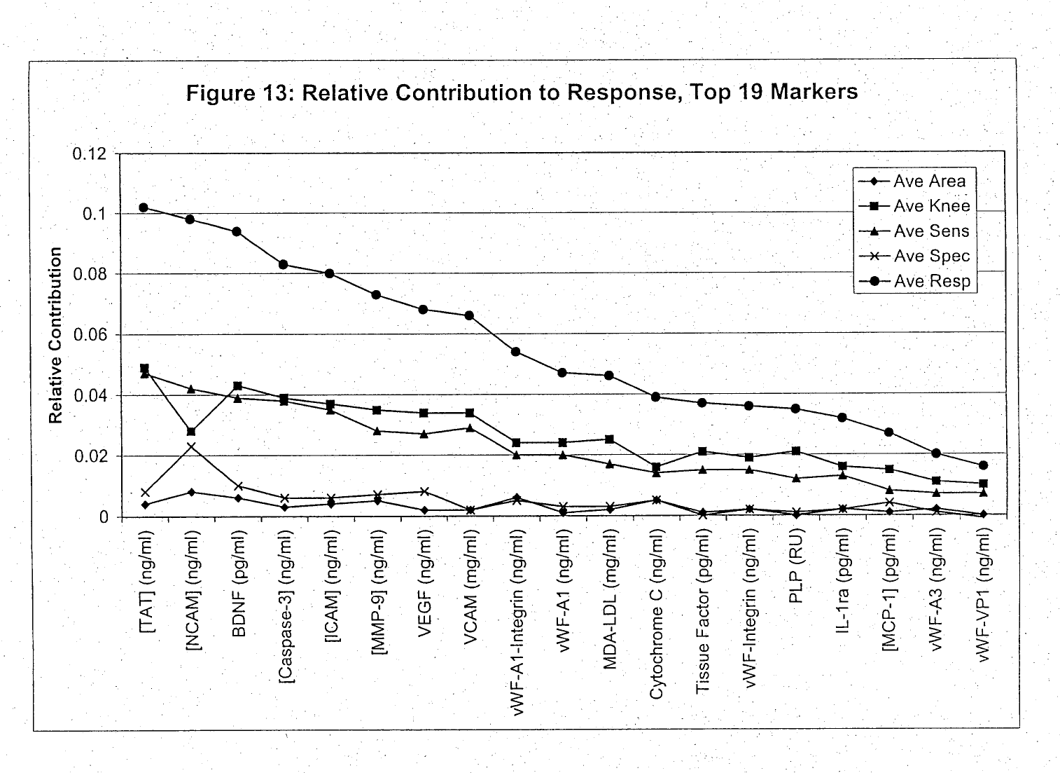

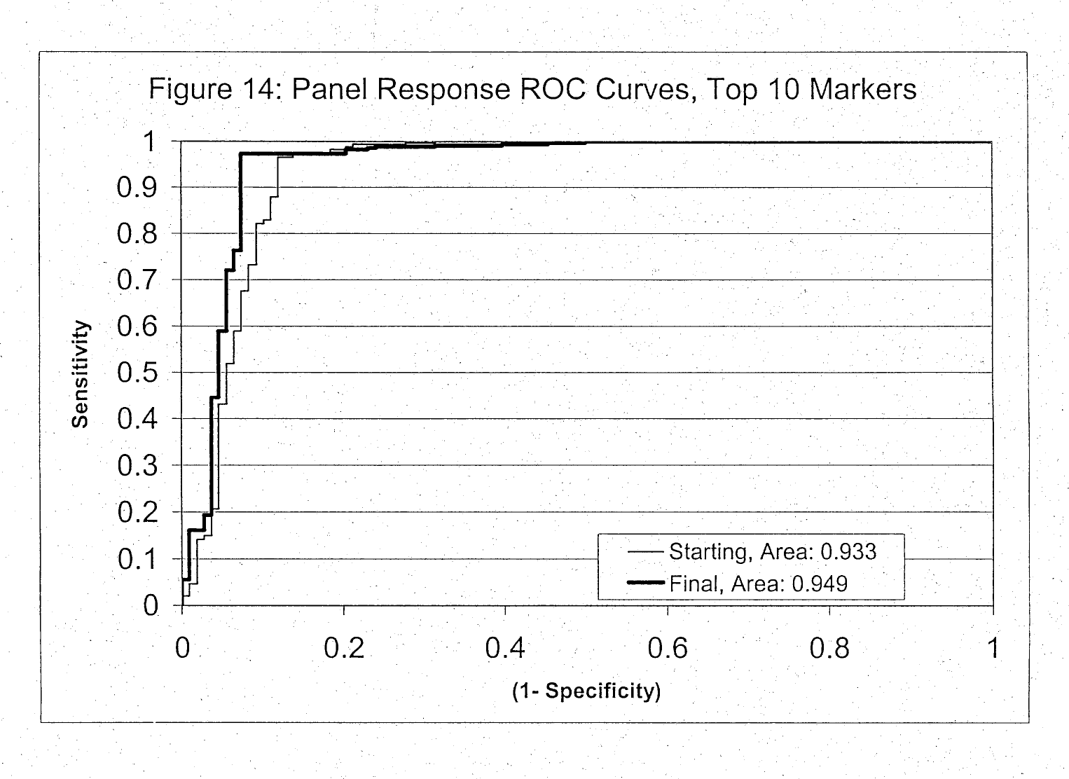

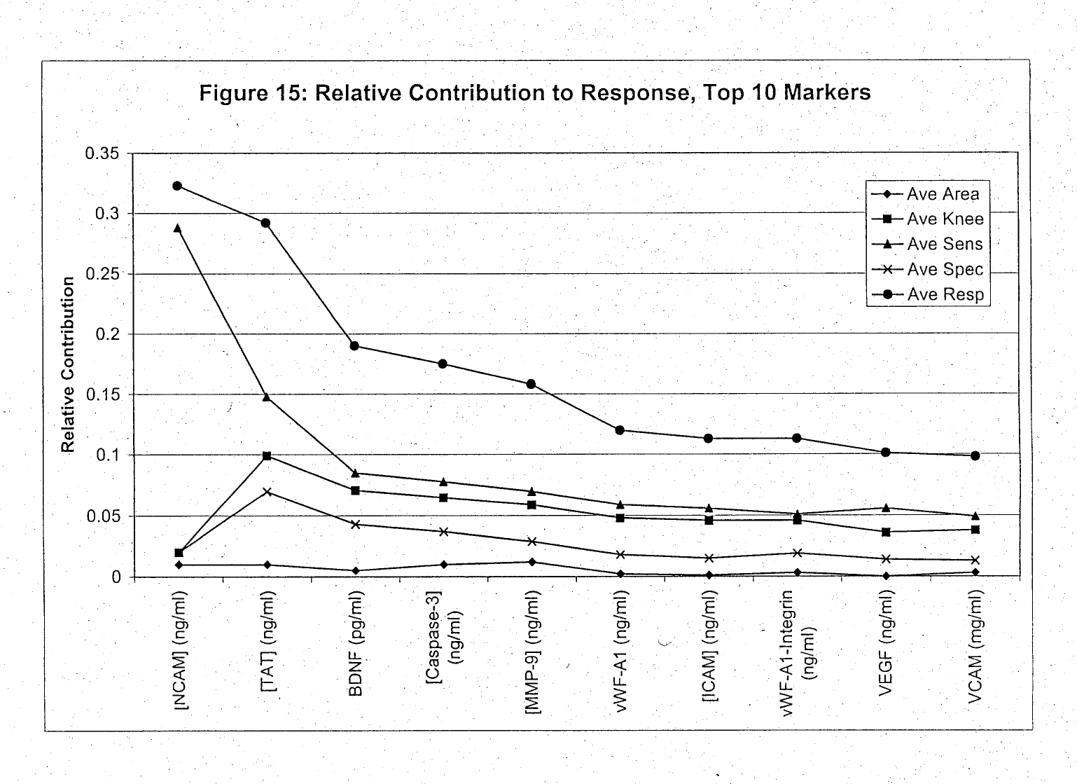

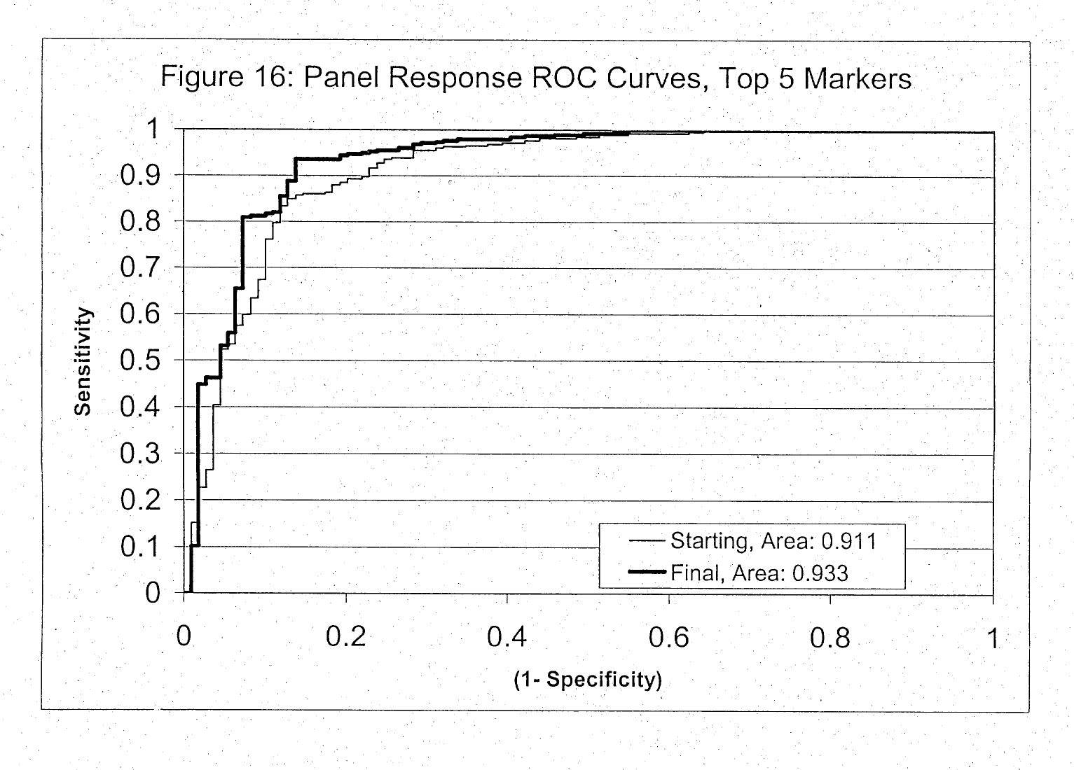

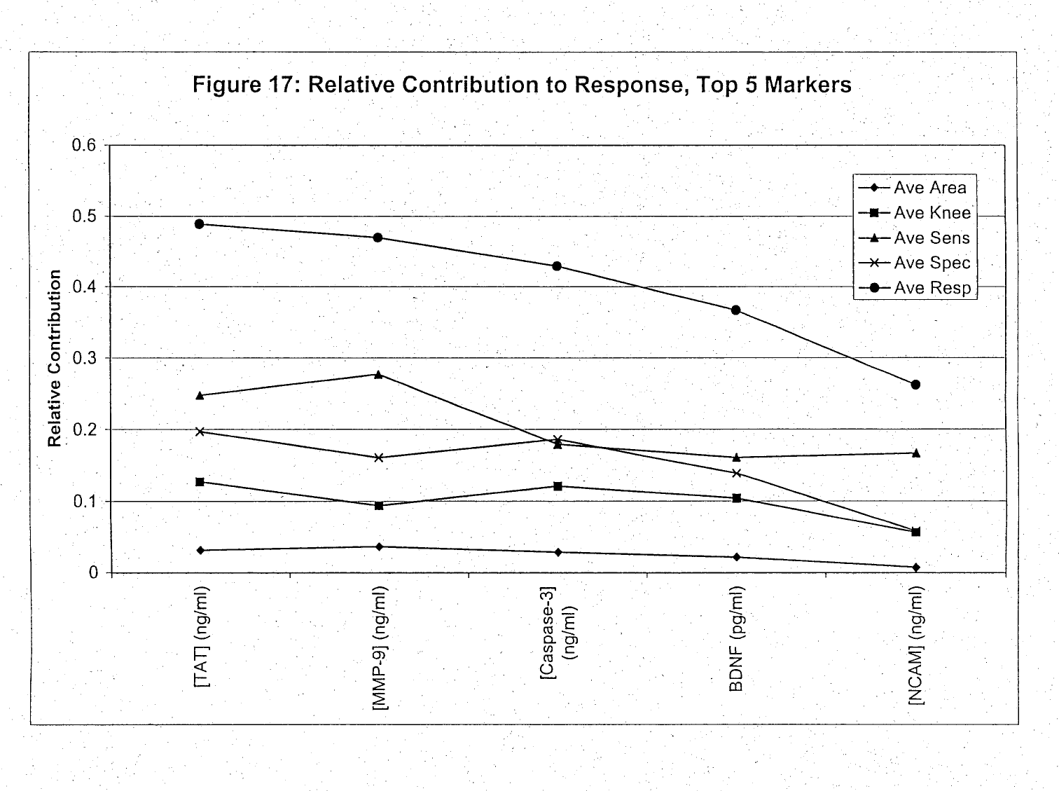

Selection of Markers for a Stroke Panel. A set of samples from patients diagnosed with stroke and normal healthy donors were assayed for several markers of potential utility. No individual marker has sufficient clinical utility to diagnose stroke. The methods described above were used to determine the optimum markers for use in a panel of markers. The data was separated into diseased and non-diseased groups. The indicator functions were selected to be ramp functions for all markers. The objective function was chosen to be the product of the area, the knee, the specificity at 92.5% sensitivity and the sensitivity at 92.5% specificity. The initial simplex was randomly distributed about a vertex derived from the univariate analysis. Using the downhill simplex method with simulated annealing a local minimum was found that maximized the objective function. For contribution for each analyte was calculated by setting the weighting parameter to zero and calculating the change in the objective function. This process was repeated 50 times. The markers were ranked by their average contribution over the 50 optimizations. The ROC curves for the initial vertex and an optimization are shown in FIG. 10. The ranking of the marker contributions is shown in FIG. 11. The lowest half of the markers were removed from the panel and the process was repeated. FIGS. 12 and 13 show the same information as in FIGS. 10 and 11 but for the 19 marker panel. The lowest 9 markers were removed from the panel and the process was repeated. FIGS. 14 and 15 show the same information as in FIGS. 10 and 11 but for the 10 marker panel. The lowest 5 markers were removed from the panel and the process was repeated a final time. FIGS. 16 and 17 show the same information as in FIGS. 10 and 11 but for the 5 marker panel. The individual ROC curves of the final 5 markers are shown in FIG. 9. The order of the contribution does not match the order of the area of the individual ROC curves. A marker with poorer univariate utility may have greater utility when used in a panel. The area of the ROC curve decreases with decreasing panel size.

### Example 2

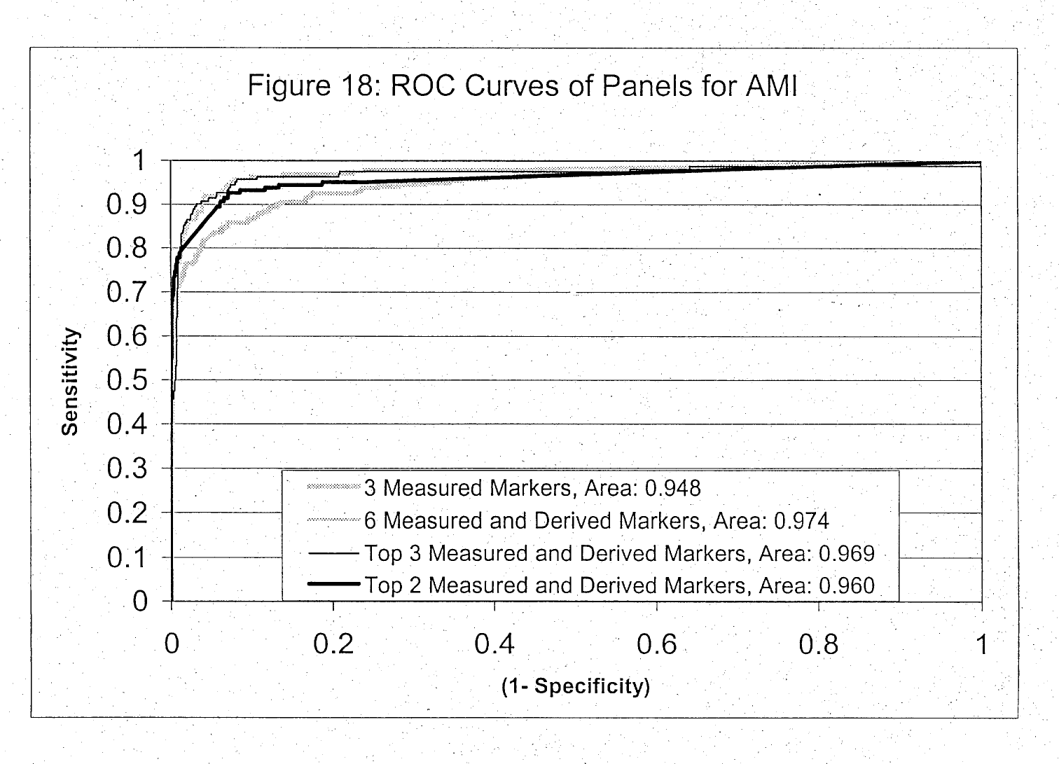

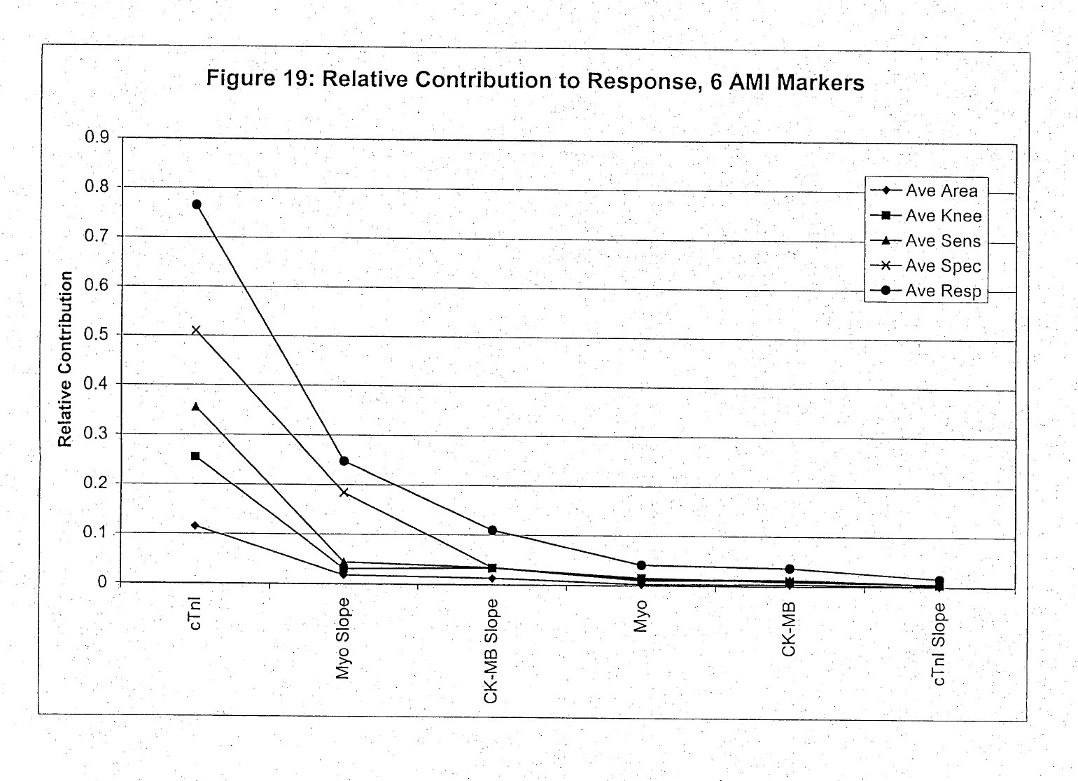

Improvement in diagnosis of AMI Utilizing Changes in Marker Levels. Data from a clinical study from patients presenting with chest pain with serial draws from each patient was analyzed using the methods described in this document. The data was first analyzed without using derived markers. The data was again analyzed utilizing derived markers that were related to the change in marker value from the initial value. The ROC curves from both optimized panel responses are shown in FIG. 18. The data clearly illustrates the utility of the change in markers to improve the diagnostic ability of panels in acute disease states. The method was also applied to determine the best 3 and 2 marker panels, and the results are also shown in FIG. 18. FIG. 19 shows the contributions of the six AMI markers. Myoglobin, while not a specific marker for AMI is a small molecule and the first marker of the three to elevate after AMI. TnI is a specific marker for AMI, but is released more slowly. The method was not aware of this but still chose TnI value and change in Myoglobin.

### Example 3

Simultaneous Optimization of Two Criteria. In this example known stroke samples are analyzed with both stroke mimics and NHD samples in the non-diseased set. There are about 50 mimics and about 500 NHD samples, so the weighting is heavily in favor of optimizing results for NHD samples. After optimization the panel response is applied to a test set stroke vs. mimics and stroke vs. NHD. Similarly the data was optimized on stroke vs mimics, and the panel response was applied to as test set stroke vs. NHD and stroke vs NHD and mimics. Table 2 shows the average results of sample runs applied to the optimization sets and then to the test sets. The effectiveness of the test is poor with respect to mimics. Two more optimizations were made as before, but this time a second group of data is simultaneously optimized. The second group consists of the stroke samples and the mimics. Table 2 also shows the average results of sample runs applied to the optimization set and when the panel response is applied to the two test sets. The effectiveness of the test with respect to mimics is now improved.

Exemplary Symptom-Based Marker Panels

Patients presenting for medical treatment often exhibit one or a few primary observable changes in bodily characteristics or functions that are indicative of disease. Often, these “symptoms” are nonspecific, in that a number of potential diseases can present the same observable symptom or symptoms. A typical list of nonspecific symptoms might include one or more of the following: shortness of breath (or dyspnea), chest pain, fever, dizziness, and headache. These symptoms can be quite common, and the number of diseases that must be considered by the clinician can be astoundingly broad.

Taking shortness of breath (referred to clinically as “dyspnea”) as an example, this symptom considered in isolation may be indicative of conditions as diverse as asthma, chronic obstructive pulmonary disease (“COPD”), tracheal stenosis, obstructive endobroncheal tumor, pulmonary fibrosis, pneumoconiosis, lymphangitic carcinomatosis, kyphoscoliosis, pleural effusion, amyotrophic lateral sclerosis, congestive heart failure, coronary artery disease, myocardial infarction, cardiomyopathy, valvular dysfunction, left ventricle hypertrophy, pericarditis, arrhythmia, pulmonary embolism, metabolic acidosis, chronic bronchitis, pneumonia, anxiety, sepsis, aneurismic dissection, etc. See, e.g., Kelley's Textbook of Internal Medicine, 4^{th} Ed., Lippincott Williams & Wilkins, Philadelphia, Pa., 2000, pp. 2349-2354, “Approach to the Patient With Dyspnea”; Mulrow et al., J. Gen. Int. Med. 8: 383-92 (1993).

Similarly, chest pain, when considered in isolation, may be indicative of stable angina, unstable angina, myocardial infarction, musculoskeletal injury, cholecystitis, gastroesophageal reflux, pulmonary embolism, pericarditis, aortic dissection, pneumonia, anxiety, etc. Moreover, the classification of chest pain as stable or unstable angina (or even mild myocardial infarction) in cases other than definitive myocardial infarction is completely subjective. The diagnosis, and in this case the distinction, is made not by angiography, which may quantify the degree of arterial occlusion, but rather by a physician's interpretation of clinical symptoms.

Differential diagnosis refers to methods for diagnosing the particular disease(s) underlying the symptoms in a particular subject, based on a comparison of the characteristic features observable from the subject to the characteristic features of those potential diseases. Depending on the breadth of diseases that must be considered in the differential diagnosis, the types and number of tests that must be ordered by a clinician can be quite large. In the case of dyspnea for example, the clinician may order tests from a group that includes radiography, electrocardiogram, exercise treadmill testing, blood chemistry analysis, echocardiography, bronchoprovocation testing, spirometry, pulse oximetry, esophageal pH monitoring, laryngoscopy, computed tomography, histology, cytology, magnetic resonance imaging, etc. See, e.g., Morgan and Hodge, Am. Fam. Physician 57: 711-16 (1998). The clinician must then integrate information obtained from a battery of tests, leading to a clinical diagnosis that most closely represents the range of symptoms and/or diagnostic test results obtained for the subject.

A first step in the identification of suitable markers for symptom-bases differential diagnosis requires a consideration of the possible diagnoses that may be causative of the non-specific symptom observed. Taking dyspnea as an example, the potential causes are myriad. The following discussion considers three potential diagnoses: congestive heart failure, pulmonary embolism, and myocardial infarction; and three potential markers for inclusion in a differential diagnosis panel for these potential diagnoses: BNP, D-dimer, and cardiac troponin.

BNP

B-type natriuretic peptide (BNP), also called brain-type natriuretic peptide is a 32 amino acid, 4 kDa peptide that is involved in the natriuresis system to regulate blood pressure and fluid balance. Bonow, R. O., Circulation 93:1946-1950 (1996). The precursor to BNP is synthesized as a 108-amino acid molecule, referred to as “pre pro BNP,” that is proteolytically processed into a 76-amino acid N-terminal peptide (amino acids 1-76), referred to as “NT pro BNP” and the 32-amino acid mature hormone, referred to as BNP or BNP 32 (amino acids 77-108). It has been suggested that each of these species—NT pro-BNP, BNP-32, and the pre pro BNP—can circulate in human plasma. Tateyama et al., Biochem. Biophys. Res. Commun. 185: 760-7 (1992); Hunt et al., Biochem. Biophys. Res. Commun. 214: 1175-83 (1995). The 2 forms, pre pro BNP and NT pro BNP, and peptides which are derived from BNP, pre pro BNP and NT pro BNP and which are present in the blood as a result of proteolyses of BNP, NT pro BNP and pre pro BNP, are collectively described as markers related to or associated with BNP.

The term “BNP” as used herein refers to the mature 32-amino acid BNP molecule itself. As the skilled artisan will recognize, however, because of its relationship to BNP, the concentration of NT pro-BNP molecule can also provide diagnostic or prognostic information in patients. The phrase “marker related to BNP or BNP related peptide” refers to any polypeptide that originates from the pre pro-BNP molecule, other than the 32-amino acid BNP molecule itself. Proteolytic degradation of BNP and of peptides related to BNP have also been described in the literature and these proteolytic fragments are also encompassed it the term “BNP related peptides.”

BNP and BNP-related peptides are predominantly found in the secretory granules of the cardiac ventricles, and are released from the heart in response to both ventricular volume expansion and pressure overload. Wilkins, M. et al., Lancet 349: 1307-10 (1997). Elevations of BNP are associated with raised atrial and pulmonary wedge pressures, reduced ventricular systolic and diastolic function, left ventricular hypertrophy, and myocardial infarction. Sagnella, G. A., Clinical Science 95: 519-29 (1998). Furthermore, there are numerous reports of elevated BNP concentration associated with congestive heart failure and renal failure. Thus, BNP levels in a patient may be indicative of several possible underlying causes of dyspnea.

D-dimer

D-dimer is a crosslinked fibrin degradation product with an approximate molecular mass of 200 kDa. The normal plasma concentration of D-dimer is <150 ng/ml (750 pM). The plasma concentration of D-dimer is elevated in patients with acute myocardial infarction and unstable angina, but not stable angina. Hoffineister, H. M. et al., Circulation 91: 2520-27 (1995); Bayes-Genis, A. et al., Thromb. Haemost. 81: 865-68 (1999); Gurfinkel, E. et al., Br. Heart J. 71: 151-55 (1994); Kruskal, J. B. et al., N. Engl. J. Med. 317: 1361-65 (1987); Tanaka, M. and Suzuki, A., Thromb. Res. 76: 289-98 (1994).

The plasma concentration of D-dimer also will be elevated during any condition associated with coagulation and fibrinolysis activation, including stroke, surgery, atherosclerosis, trauma, and thrombotic thrombocytopenic purpura. D-dimer is released into the bloodstream immediately following proteolytic clot dissolution by plasmin. The plasma concentration of D-dimer can exceed 2 μg/ml in patients with unstable angina. Gurfinkel, E. et al., Br. Heart J. 71: 151-55 (1994). Plasma D-dimer is a specific marker of fibrinolysis and indicates the presence of a prothrombotic state associated with acute myocardial infarction and unstable angina. The plasma concentration of D-dimer is also nearly always elevated in patients with acute pulmonary embolism; thus, normal levels of D-dimer may allow the exclusion of pulmonary embolism. Egermayer et al., Thorax 53: 830-34 (1998).

Cardiac Troponin

Troponin I (TnI) is a 25 kDa inhibitory element of the troponin complex, found in muscle tissue. TnI binds to actin in the absence of Ca²⁺, inhibiting the ATPase activity of actomyosin. A TnI isoform that is found in cardiac tissue (cTnI) is 40% divergent from skeletal muscle TnI, allowing both isoforms to be immunologically distinguished. The normal plasma concentration of cTnI is <0.1 ng/ml (4 pM). cTnI is released into the bloodstream following cardiac cell death; thus, the plasma cTnI concentration is elevated in patients with acute myocardial infarction. Investigations into changes in the plasma cTnI concentration in patients with unstable angina have yielded mixed results, but cTnI is not elevated in the plasma of individuals with stable angina. Benamer, H. et al., Am. J. Cardiol. 82: 845-50 (1998); Bertinchant, J. P. et al., Clin. Biochem. 29: 587-94 (1996); Tanasijevic, M. J. et al., Clin. Cardiol. 22: 13-16 (1999); Musso, P. et al., J. Ital. Cardiol. 26: 1013-23 (1996); Holvoet, P. et al., JAMA 281: 1718-21 (1999); Holvoet, P. et al., Circulation 98: 1487-94 (1998).

The plasma concentration of cTnI in patients with acute myocardial infarction is significantly elevated 4-6 hours after onset, peaks between 12-16 hours, and can remain elevated for one week. The release kinetics of cTnI associated with unstable angina may be similar. The measurement of specific forms of cardiac troponin, including free cardiac troponin I and complexes of cardiac troponin I with troponin C and/or T may provide the user with the ability to identify various stages of ACS. Free and complexed cardiac-troponin T may be used in a manner analogous to that described for cardiac troponin I. Cardiac troponin T complex may be useful either alone or when expressed as a ratio with total cardiac troponin I to provide information related to the presence of progressing myocardial damage. Ongoing ischemia may result in the release of the cardiac troponin TIC complex, indicating that higher ratios of cardiac troponin TIC:total cardiac troponin I may be indicative of continual damage caused by unresolved ischemia. See, U.S. Pat. Nos. 6,147,688, 6,156,521, 5,947,124, and 5,795,725.

Based on the foregoing discussion, the skilled artisan will recognize that, for example, increased BNP is indicative of congestive heart failure, but may also be indicative of other cardiac-related conditions such as myocardial infarction. Thus, the inclusion of a marker related to myocardial injury such as cardiac troponin I and/or cardiac troponin T can permit further discrimination of the disease underlying the observed dyspnea and the increased BNP level. In this case, an increased level of cardiac troponin may be used to rule in myocardial infarction.

Similarly, BNP may also be indicative of pulmonary embolism. The inclusion of a marker related to coagulation and hemostasis such as D-dimer can permit further discrimination of the disease underlying the observed dyspnea and the increased BNP level. In this case, a normal level of D-dimer may be used to rule out pulmonary embolism.

The skilled artisan will readily acknowledge that other markers may be substituted in or added to this marker panel to further discriminate the causes of dyspnea. Additional suitable markers are described in the following sections.

(i) Markers Related To Myocardial Injury

Annexin V, also called lipocortin V, endonexin II, calphobindin I, calcium binding protein 33, placental anticoagulant protein I, thromboplastin inhibitor, vascular anticoagulant-α, and anchorin CII, is a 33 kDa calcium-binding protein that is an indirect inhibitor and regulator of tissue factor. Annexin V is composed of four homologous repeats with a consensus sequence common to all annexin family members, binds calcium and phosphatidyl serine, and is expressed in a wide variety of tissues, including heart, skeletal muscle, liver, and endothelial cells (Giambanco, I. et al., J. Histochem. Cytochem. 39:P1189-1198, 1991; Doubell, A. F. et al., Cardiovasc. Res. 27:1359-1367, 1993). The normal plasma concentration of annexin V is <2 ng/ml (Kaneko, N. et al., Clin. Chim. Acta 251:65-80, 1996). The plasma concentration of annexin V is elevated in individuals with acute myocardial infarction (Kaneko, N. et al., Clin. Chim. Acta 251:65-80, 1996). Due to its wide tissue distribution, elevation of the plasma concentration of annexin V may be associated with any condition involving non-cardiac tissue injury. However, one study has found that plasma annexin V concentrations were not significantly elevated in patients with old myocardial infarction, chest pain syndrome, valvular heart disease, lung disease, and kidney disease (Kaneko, N. et al., Clin. Chim. Acta 251:65-80, 1996). Annexin V is released into the bloodstream soon after acute myocardial infarction onset. The annexin V concentration in the plasma of acute myocardial infarction patients decreased from initial (admission) values, suggesting that it is rapidly cleared from the bloodstream (Kaneko, N. et al. Clin. Chim. Acta 251:65-80, 1996).

Enolase is a 78 kDa homo- or heterodimeric cytosolic protein produced from α, β, and γ subunits. Enolase catalyzes the interconversion of 2-phosphoglycerate and phosphoenolpyruvate in the glycolytic pathway. Enolase is present as αα, αβ, ββ, αγ, and γγ isoforms. The α subunit is found in most tissues, the β subunit is found in cardiac and skeletal muscle, and the γ subunit is found primarily in neuronal and neuroendocrine tissues. β-enolase is composed of αβ and ββ enolase, and is specific for muscle. The normal plasma concentration of β-enolase is <10 ng/ml (120 pM). β-enolase is elevated in the serum of individuals with acute myocardial infarction, but not in individuals with angina (Nomura, M. et al., Br. Heart J. 58:29-33, 1987; Herraez-Dominguez, M. V. et al., Clin. Chim. Acta 64:307-315, 1975). Further investigations into possible changes in plasma β-enolase concentration associated with unstable and stable angina need to be performed. The plasma concentration of β-enolase is elevated during heart surgery, muscular dystrophy, and skeletal muscle injury (Usui, A. et al., Cardiovasc. Res. 23:737-740, 1989; Kato, K. et al., Clin. Chim. Acta 131:75-85, 1983; Matsuda, H. et al., Forensic Sci. Int. 99:197-208, 1999). β-enolase is released into the bloodstream immediately following cardiac or skeletal muscle injury. The plasma β-enolase concentration was elevated to more than 150 ng/ml in the perioperative stage of cardiac surgery, and remained elevated for 1 week. Serum β-enolase concentrations peaked approximately 12-14 hours after the onset of chest pain and acute myocardial infarction and approached baseline after 1 week had elapsed from onset, with maximum levels approaching 1 μg/ml (Kato, K. et al., Clin. Chim. Acta 131:75-85, 1983; Nomura, M. et al., Br. Heart J. 58:29-33, 1987).

Creatine kinase (CK) is a 85 kDa cytosolic enzyme that catalyzes the reversible formation ADP and phosphocreatine from ATP and creatine. CK is a homo- or heterodimer composed of M and B chains. CK-MB is the isoform that is most specific for cardiac tissue, but it is also present in skeletal muscle and other tissues. The normal plasma concentration of CK-MB is <5 ng/ml. The plasma CK-MB concentration is significantly elevated in patients with acute myocardial infarction. Plasma CK-MB is not elevated in patients with stable angina, and investigation into plasma CK-MB concentration elevations in patients with unstable angina have yielded mixed results (Thygesen, K. et al., Eur. J. Clin. Invest. 16:1-4, 1986; Koukkunen, H. et al., Ann. Med. 30:488-496, 1998; Bertinchant, J. P. et al., Clin. Biochem. 29:587-594, 1996; Benamer, H. et al., Am. J. Cardiol. 82:845-850, 1998; Norregaard-Hansen, K. et al., Eur. Heart J. 13:188-193, 1992). The mixed results associated with unstable angina suggest that CK-MB may be useful in determining the severity of unstable angina because the extent of myocardial ischemia is directly proportional to unstable angina severity. Elevations of the plasma CK-MB concentration are associated with skeletal muscle injury and renal disease. CK-MB is released into the bloodstream following cardiac cell death. The plasma concentration of CK-MB in patients with acute myocardial infarction is significantly elevated 4-6 hours after onset, peaks between 12-24 hours, and returns to baseline after 3 days. The release kinetics of CK-MB associated with unstable angina may be similar.

Glycogen phosphorylase (GP) is a 188 kDa intracellular allosteric enzyme that catalyzes the removal of glucose (liberated as glucose-I-phosphate) from the nonreducing ends of glycogen in the presence of inorganic phosphate during glycogenolysis. GP is present as a homodimer, which associates with another homodimer to form a tetrameric enzymatically active phosphorylase A. There are three isoforms of GP that can be immunologically distinguished. The BB isoform is found in brain and cardiac tissue, the MM isoform is found in skeletal muscle and cardiac tissue, and the LL isoform is predominantly found in liver (Mair, J. et al., Br. Heart J. 72:125-127, 1994). GP-BB is normally associated with the sarcoplasmic reticulum glycogenolysis complex, and this association is dependent upon the metabolic state of the myocardium (Mair, J., Clin. Chim. Acta 272:79-86, 1998). At the onset of hypoxia, glycogen is broken down, and GP-BB is converted from a bound form to a free cytoplasmic form (Krause, E. G. et al. Mol. Cell Biochem. 160-161:289-295, 1996). The normal plasma GP-BB concentration is <7 ng/ml (36 pM). The plasma GP-BB concentration is significantly elevated in patients with acute myocardial infarction and unstable angina with transient ST-T elevations, but not stable angina (Mair, J. et al., Br. Heart J. 72:125-127, 1994; Mair, J., Clin. Chim. Acta 272:79-86, 1998; Rabitzsch, G. et al., Clin. Chem. 41:966-978, 1995; Rabitzsch, G. et al., Lancet 341:1032-1033, 1993). Furthermore, GP-BB also can be used to detect perioperative acute myocardial infarction and myocardial ischemia in patients undergoing coronary artery bypass surgery (Rabitzsch, G. et al., Biomed. Biochim. Acta 46:S584-S588, 1987; Mair, P. et al., Eur. J. Clin. Chem. Clin. Biochem. 32:543-547, 1994). GP-BB has been demonstrated to be a more sensitive marker of unstable angina and acute myocardial infarction early after onset than CK-MB, cardiac tropopnin T, and myoglobin (Rabitzsch, G. et al., Clin. Chem. 41:966-978, 1995). Because it is also found in the brain, the plasma GP-BB concentration also may be elevated during ischemic cerebral injury. GP-BB is released into the bloodstream under ischemic conditions that also involve an increase in the permeability of the cell membrane, usually a result of cellular necrosis. GP-BB is significantly elevated within 4 hours of chest pain onset in individuals with unstable angina and transient ST-T ECG alterations, and is significantly elevated while myoglobin, CK-MB, and cardiac troponin T are still within normal levels (Mair, J. et al., Br. Heart J. 72:125-127, 1994). Furthermore, GP-BB can be significantly elevated 1-2 hours after chest pain onset in patients with acute myocardial infarction (Rabitzsch, G. et al., Lancet 341:1032-1033, 1993). The plasma GP-BB concentration in patients with unstable angina and acute myocardial infarction can exceed 50 ng/ml (250 pM) (Mair, J. et al., Br. Heart J. 72:125-127, 1994; Mair, J., Clin. Chim. Acta 272:79-86, 1998; Krause, E. G. et al., Mol. Cell Biochem. 160-161:289-295, 1996; Rabitzsch, G. et al., Clin. Chem. 41:966-978, 1995; Rabitzsch, G. et al., Lancet 341:1032-1033, 1993). GP-BB appears to be a very sensitive marker of myocardial ischemia, with specificity similar to that of CK-BB. GP-BB plasma concentrations are elevated within the first 4 hours after acute myocardial infarction onset, which suggests that it may be a very useful early marker of myocardial damage. Furthermore, GP-BB is not only a more specific marker of cardiac tissue damage, but also ischemia, since it is released to an unbound form during cardiac ischemia and would not normally be released upon traumatic injury. This is best illustrated by the usefulness of GP-BB in detecting myocardial ischemia during cardiac surgery. GP-BB may be a very useful marker of early myocardial ischemia during acute myocardial infarction and severe unstable angina.

Heart-type fatty acid binding protein (H-FABP) is a cytosolic 15 kDa lipid-binding protein involved in lipid metabolism. Heart-type FABP antigen is found not only in heart tissue, but also in kidney, skeletal muscle, aorta, adrenals, placenta, and brain (Veerkamp, J. H. and Maatman, R. G., Prog. Lipid Res. 34:17-52, 1995; Yoshimoto, K. et al., Heart Vessels 10:304-309, 1995). Furthermore, heart-type FABP mRNA can be found in testes, ovary, lung, mammary gland, and stomach (Veerkamp, J. H. and Maatman, R. G., Prog. Lipid Res. 34:17-52, 1995). The normal plasma concentration of FABP is <6 ng/ml (400 pM). The plasma H-FABP concentration is elevated in patients with acute myocardial infarction and unstable angina (Ishii, J. et al., Clin. Chem. 43:1372-1378, 1997; Tsuji, R. et al., Int. J. Cardiol. 41:209-217, 1993). Furthermore, H-FABP may be useful in estimating infarct size in patients with acute myocardial infarction (Glatz, J. F. et al., Br. Heart J. 71:135-140, 1994). Myocardial tissue as a source of H-FABP can be confirmed by determining the ratio of myoglobin/FABP (grams/grams). A ratio of approximately 5 indicates that FABP is of myocardial origin, while a higher ratio indicates skeletal muscle sources (Van Nieuwenhoven, F. A. et al., Circulation 92:2848-2854, 1995). Because of the presence of H-FABP in skeletal muscle, kidney and brain, elevations in the plasma H-FABP concentration may be associated with skeletal muscle injury, renal disease, or stroke. H-FABP is released into the bloodstream following cardiac tissue necrosis. The plasma H-FABP concentration can be significantly elevated 1-2 hours after the onset of chest pain, earlier than CK-MB and myoglobin (Tsuji, R. et al., Int. J. Cardiol. 41:209-217, 1993; Van Nieuwenhoven, F. A. et al., Circulation 92:2848-2854, 1995; Tanaka, T. et al., Clin. Biochem. 24:195-201, 1991). Additionally, H-FABP is rapidly cleared from the bloodstream, and plasma concentrations return to baseline after 24 hours after acute myocardial infarction onset (Glatz, J. F. et al., Br. Heart J. 71:135-140, 1994; Tanaka, T. et al., Clin. Biochem. 24:195-201, 1991).

Phosphoglyceric acid mutase (PGAM) is a 57 kDa homo- or heterodimeric intracellular glycolytic enzyme composed of 29 kDa M or B subunits that catalyzes the interconversion of 3-phosphoglycerate to 2-phosphoglycerate in the presence of magnesium. Cardiac tissue contains isozymes MM, MB, and BB, skeletal muscle contains primarily PGAM-MM, and most other tissues contain PGAM-BB (Durany, N. and Carreras, J., Comp. Biochem. Physiol. B. Biochem. Mol. Biol. 114:217-223, 1996). Thus, PGAM-MB is the most specific isozyme for cardiac tissue. PGAM is elevated in the plasma of patients with acute myocardial infarction, but further studies need to be performed to determine changes in the plasma PGAM concentration associated with acute myocardial infarction, unstable angina and stable angina (Mair, J., Crit. Rev. Clin. Lab. Sci. 34:1-66, 1997). Plasma PGAM-MB concentration elevations may be associated with unrelated myocardial or possibly skeletal tissue damage. PGAM-MB is most likely released into the circulation following cellular necrosis. PGAM has a half-life of less than 2 hours in the bloodstream of rats (Grisolia, S. et al., Physiol. Chem. Phys. 8:37-52, 1976).

S-100 is a 21 kDa homo- or heterodimeric cytosolic Ca²⁺-binding protein produced from α and β subunits. It is thought to participate in the activation of cellular processes along the Ca²⁺-dependent signal transduction pathway (Bonfrer, J. M. et al., Br. J. Cancer 77:2210-2214, 1998). S-100ao (αα isoform) is found in striated muscles, heart and kidney, S-100a (αβ isoform) is found in glial cells, but not in Schwann cells, and S-100b (ββ isoform) is found in high concentrations in glial cells and Schwann cells, where it is a major cytosolic component (Kato, K. and Kimura, S., Biochim. Biophys. Acta 842:146-150, 1985; Hasegawa, S. et al., Eur. Urol. 24:393-396, 1993). The normal serum concentration of S-100ao is <0.25 ng/ml (12 pM), and its concentration may be influenced by age and sex, with higher concentrations in males and older individuals (Kikuchi, T. et al., Hinyokika Kiyo 36:1117-1123, 1990; Morita, T. et al., Nippon Hinyokika Gakkai Zasshi 81:1162-1167, 1990; Usui, A. et al., Clin. Chem. 36:639-641, 1990). The serum concentration of S-100ao is elevated in patients with acute myocardial infarction, but not in patients with angina pectoris with suspected acute myocardial infarction (Usui, A. et al., Clin. Chem. 36:639-641, 1990). Further investigation is needed to determine changes in the plasma concentration of S-100ao associated with unstable and stable angina. Serum S-100ao is elevated in the serum of patients with renal cell carcinoma, bladder tumor, renal failure, and prostate cancer, as well as in patients undergoing open heart surgery (Hasegawa, S. et al., Eur. Urol. 24:393-396, 1993; Kikuchi, T. et al., Hinyokika Kiyo 36:1117-1123, 1990; Morita, T. et al., Nippon Hinyokika Gakkai Zasshi 81:1162-1167, 1990; Usui, A. et al., Clin. Chem. 35:1942-1944, 1989). S-100ao is a cytosolic protein that will be released into the extracellular space following cell death. The serum concentration of S-100ao is significantly elevated on admission in patients with acute myocardial infarction, increases to peak levels 8 hours after admission, decreases and returns to baseline one week later (Usui, A. et al., Clin. Chem. 36:639-641, 1990). Furthermore, S-100ao appears to be significantly elevated earlier after acute myocardial infarction onset than CK-MB (Usui, A. et al., Clin. Chem. 36:639-641, 1990). The maximum serum S-100ao concentration can exceed 100 ng/ml. S-100ao may be rapidly cleared from the bloodstream by the kidney, as suggested by the rapid decrease of the serum S-100ao concentration of heart surgery patients following reperfusion and its increased urine concentration. S-100ao is found in high concentration in cardiac tissue and appears to be a sensitive marker of cardiac injury. Major sources of non-specificity of this marker include skeletal muscle and renal tissue injury. S-100ao may be significantly elevated soon after acute myocardial infarction onset, and it may allow for the discrimination of acute myocardial infarction from unstable angina. Patients with angina pectoris and suspected acute myocardial infarction, indicating that they were suffering chest pain associated with an ischemic episode, did not have a significantly elevated S-100ao concentration.

(ii) Additional Markers Related to Coagulation and Hemostasis

Plasmin is a 78 kDa serine proteinase that proteolytically digests crosslinked fibrin, resulting in clot dissolution. The 70 kDa serine proteinase inhibitor α2-antiplasmin (α2AP) regulates plasmin activity by forming a covalent 1:1 stoichiometric complex with plasmin. The resulting ˜150 kDa plasmin-α2AP complex (PAP), also called plasmin inhibitory complex (PIC) is formed immediately after α2AP comes in contact with plasmin that is activated during fibrinolysis. The normal serum concentration of PAP is <1 μg/ml (6.9 nM). Elevations in the serum concentration of PAP can be attributed to the activation of fibrinolysis. Elevations in the serum concentration of PAP may be associated with clot presence, or any condition that causes or is a result of fibrinolysis activation. These conditions can include atherosclerosis, disseminated intravascular coagulation, acute myocardial infarction, surgery, trauma, unstable angina, stroke, and thrombotic thrombocytopenic purpura. PAP is formed immediately following proteolytic activation of plasmin. PAP is a specific marker for fibrinolysis activation and the presence of a recent or continual hypercoagulable state.

β-thromboglobulin (βTG) is a 36 kDa platelet α granule component that is released upon platelet activation. The normal plasma concentration of βTG is <40 ng/ml (1.1 nM). Plasma levels of β-TG appear to be elevated in patients with unstable angina and acute myocardial infarction, but not stable angina (De Caterina, R. et al., Eur. Heart J. 9:913-922, 1988; Bazzan, M. et al., Cardiologia 34, 217-220, 1989). Plasma β-TG elevations also seem to be correlated with episodes of ischemia in patients with unstable angina (Sobel, M. et al., Circulation 63:300-306, 1981). Elevations in the plasma concentration of PTG may be associated with clot presence, or any condition that causes platelet activation. These conditions can include atherosclerosis, disseminated intravascular coagulation, surgery, trauma, and thrombotic thrombocytopenic purpura, and stroke (Landi, G. et al., Neurology 37:1667-1671, 1987). PTG is released into the circulation immediately after platelet activation and aggregation. It has a biphasic half-life of 10 minutes, followed by an extended 1 hour half-life in plasma (Switalska, H. I. et al., J. Lab. Clin. Med. 106:690-700, 1985). Plasma βTG concentration is reportedly elevated dring unstable angina and acute myocardial infarction. Special precautions must be taken to avoid platelet activation during the blood sampling process. Platelet activation is common during regular blood sampling, and could lead to artificial elevations of plasma βPTG concentration. In addition, the amount of βPTG released into the bloodstream is dependent on the platelet count of the individual, which can be quite variable. Plasma concentrations of PTG associated with ACS can approach 70 ng/ml (2 nM), but this value may be influenced by platelet activation during the sampling procedure.

Platelet factor 4 (PF4) is a 40 kDa platelet α granule component that is released upon platelet activation. PF4 is a marker of platelet activation and has the ability to bind and neutralize heparin. The normal plasma concentration of PF4 is <7 ng/ml (175 pM). The plasma concentration of PF4 appears to be elevated in patients with acute myocardial infarction and unstable angina, but not stable angina (Gallino, A. et al., Am. Heart J. 112:285-290, 1986; Sakata, K. et al., Jpn. Circ. J. 60:277-284, 1996; Bazzan, M. et al., Cardiologia 34:217-220, 1989). Plasma PF4 elevations also seem to be correlated with episodes of ischemia in patients with unstable angina (Sobel, M. et al., Circulation 63:300-306, 1981). Elevations in the plasma concentration of PF4 may be associated with clot presence, or any condition that causes platelet activation. These conditions can include atherosclerosis, disseminated intravascular coagulation, surgery, trauma, thrombotic thrombocytopenic purpura, and acute stroke (Carter, A. M. et al., Arterioscler. Thromb. Vasc. Biol. 18:1124-1131, 1998). PF4 is released into the circulation immediately after platelet activation and aggregation. It has a biphasic half-life of 1 minute, followed by an extended 20 minute half-life in plasma. The half-life of PF4 in plasma can be extended to 20-40 minutes by the presence of heparin (Rucinski, B. et al., Am. J. Physiol. 251:H800-H807, 1986). Plasma PF4 concentration is reportedly elevated during unstable angina and acute myocardial infarction, but these studies may not be completely reliable. Special precautions must be taken to avoid platelet activation during the blood sampling process. Platelet activation is common during regular blood sampling, and could lead to artificial elevations of plasma PF4 concentration. In addition, the amount of PF4 released into the bloodstream is dependent on the platelet count of the individual, which can be quite variable. Plasma concentrations of PF4 associated with disease can exceed 100 ng/ml (2.5 nM), but it is likely that this value may be influenced by platelet activation during the sampling procedure.

Fibrinopeptide A (FPA) is a 16 amino acid, 1.5 kDa peptide that is liberated from amino terminus of fibrinogen by the action of thrombin. Fibrinogen is synthesized and secreted by the liver. The normal plasma concentration of FPA is <5 ng/ml (3.3 nM). The plasma FPA concentration is elevated in patients with acute myocardial infarction, unstable angina, and variant angina, but not stable angina (Gensini, G. F. et al., Thromb. Res. 50:517-525, 1988; Gallino, A. et al., Am. Heart J. 112:285-290, 1986; Sakata, K. et al., Jpn. Circ. J. 60:277-284, 1996; Theroux, P. et al., Circulation 75:156-162, 1987; Merlini, P. A. et al., Circulation 90:61-68, 1994; Manten, A. et al., Cardiovasc. Res. 40:389-395, 1998). Furthermore, plasma FPA may indicate the severity of angina (Gensini, G. F. et al., Thromb. Res. 50:517-525, 1988). Elevations in the plasma concentration of FPA are associated with any condition that involves activation of the coagulation pathway, including stroke, surgery, cancer, disseminated intravascular coagulation, nephrosis, and thrombotic thrombocytopenic purpura. FPA is released into the circulation following thrombin activation and cleavage of fibrinogen. Because FPA is a small polypeptide, it is likely cleared from the bloodstream rapidly. FPA has been demonstrated to be elevated for more than one month following clot formation, and maximum plasma FPA concentrations can exceed 40 ng/ml in active angina (Gensini, G. F. et al., Thromb. Res. 50:517-525, 1988; Tohgi, H. et al., Stroke 21:1663-1667, 1990).

Platelet-derived growth factor (PDGF) is a 28 kDa secreted homo- or heterodimeric protein composed of the homologous subunits A and/or B (Mahadevan, D. et al., J. Biol. Chem. 270:27595-27600, 1995). PDGF is a potent mitogen for mesenchymal cells, and has been implicated in the pathogenesis of atherosclerosis. PDGF is released by aggregating platelets and monocytes near sites of vascular injury. The normal plasma concentration of PDGF is <0.4 ng/ml (15 pM). Plasma PDGF concentrations are higher in individuals with acute myocardial infarction and unstable angina than in healthy controls or individuals with stable angina (Ogawa, H. et al., Am. J. Cardiol. 69:453-456, 1992; Wallace, J. M. et al., Ann. Clin. Biochem. 35:236-241, 1998; Ogawa, H. et al., Coron. Artery Dis. 4:437-442, 1993). Changes in the plasma PDGF concentration in these individuals is most likely due to increased platelet and monocyte activation. Plasma PDGF is elevated in individuals with brain tumors, breast cancer, and hypertension (Kurimoto, M. et al., Acta Neurochir. (Wien) 137:182-187, 1995; Seymour, L. et al., Breast Cancer Res. Treat. 26:247-252, 1993; Rossi, E. et al., Am. J. Hypertens. 11:1239-1243, 1998). Plasma PDGF may also be elevated in any pro-inflammatory condition or any condition that causes platelet activation including surgery, trauma, disseminated intravascular coagulation, and thrombotic thrombocytopenic purpura. PDGF is released from the secretory granules of platelets and monocytes upon activation. PDGF has a biphasic half-life of approximately 5 minutes and 1 hour in animals (Cohen, A. M. et al., J. Surg. Res. 49:447-452, 1990; Bowen-Pope, D. F. et al., Blood 64:458-469, 1984). The plasma PDGF concentration in ACS can exceed 0.6 ng/ml (22 pM) (Ogawa, H. et al., Am. J. Cardiol. 69:453-456, 1992). PDGF may be a sensitive and specific marker of platelet activation. In addition, it may be a sensitive marker of vascular injury, and the accompanying monocyte and platelet activation.

Prothrombin fragment 1+2 is a 32 kDa polypeptide that is liberated from the amino terminus of thrombin during thrombin activation. The normal plasma concentration of F1+2 is <32 ng/ml (1 nM). The plasma concentration of F1+2 is reportedly elevated in patients with acute myocardial infarction and unstable angina, but not stable angina, but the changes were not robust (Merlini, P. A. et al., Circulation 90:61-68, 1994). Other reports have indicated that there is no significant change in the plasma F1+2 concentration in cardiovascular disease (Biasucci, L. M. et al., Circulation 93:2121-2127, 1996; Manten, A. et al., Cardiovasc. Res. 40:389-395, 1998). The concentration of F1+2 in plasma can be elevated during any condition associated with coagulation activation, including stroke, surgery, trauma, thrombotic thrombocytopenic purpura, and disseminated intravascular coagulation. F 1+2 is released into the bloodstream immediately upon thrombin activation. F1+2 has a half-life of approximately 90 minutes in plasma, and it has been suggested that this long half-life may mask bursts of thrombin formation (Biasucci, L. M. et al., Circulation 93:2121-2127, 1996).

P-selectin, also called granule membrane protein-140, GMP-140, PADGEM, and CD-62P, is a ˜140 kDa adhesion molecule expressed in platelets and endothelial cells. P-selectin is stored in the alpha granules of platelets and in the Weibel-Palade bodies of endothelial cells. Upon activation, P-selectin is rapidly translocated to the surface of endothelial cells and platelets to facilitate the “rolling” cell surface interaction with neutrophils and monocytes. Membrane-bound and soluble forms of P-selectin have been identified. Soluble P-selectin may be produced by shedding of membrane-bound P-selectin, either by proteolysis of the extracellular P-selectin molecule, or by proteolysis of components of the intracellular cytoskeleton in close proximity to the surface-bound P-selectin molecule (Fox, J. E., Blood Coagul. Fibrinolysis 5:291-304, 1994). Additionally, soluble P-selectin may be translated from mRNA that does not encode the N-terminal transmembrane domain (Dunlop, L. C. et al., J. Exp. Med. 175:1147-1150, 1992; Johnston, G. I. et al., J. Biol. Chem. 265:21381-21385, 1990). Activated platelets can shed membrane-bound P-selectin and remain in the circulation, and the shedding of P-selectin can elevate the plasma P-selectin concentration by approximately 70 ng/ml (Michelson, A. D. et al., Proc. Natl. Acad. Sci. U.S. A. 93:11877-11882, 1996). Soluble P-selectin may also adopt a different conformation than membrane-bound P-selectin. Soluble P-selectin has a monomeric rod-like structure with a globular domain at one end, and the membrane-bound molecule forms rosette structures with the globular domain facing outward (Ushiyama, S. et al., J. Biol. Chem. 268:15229-15237, 1993). Soluble P-selectin may play an important role in regulating inflammation and thrombosis by blocking interactions between leukocytes and activated platelets and endothelial cells (Gamble, J. R. et al., Science 249:414-417, 1990). The normal plasma concentration of soluble P-selectin is <200 ng/ml. Blood is normally collected using citrate as an anticoagulant, but some studies have used EDTA plasma with additives such as prostaglandin E to prevent platelet activation. EDTA may be a suitable anticoagulant that will yield results comparable to those obtained using citrate. Furthermore, the plasma concentration of soluble P-selectin may not be affected by potential platelet activation during the sampling procedure. The plasma soluble P-selectin concentration was significantly elevated in patients with acute myocardial infarction and unstable angina, but not stable angina, even following an exercise stress test (Ikeda, H. et al., Circulation 92:1693-1696, 1995; Tomoda, H. and Aoki, N., Angiology 49:807-813, 1998; Hollander, J. E. et al., J. Am. Coil. Cardiol. 34:95-105, 1999; Kaikita, K. et al., Circulation 92:1726-1730, 1995; Ikeda, H. et al., Coron. Artery Dis. 5:515-518, 1994). The sensitivity and specificity of membrane-bound P-selectin versus soluble P-selectin for acute myocardial infarction is 71% versus 76% and 32% versus 45% (Hollander, J. E. et al., J. Am. Coll. Cardiol. 34:95-105, 1999). The sensitivity and specificity of membrane-bound P-selectin versus soluble P-selectin for unstable angina+acute myocardial infarction is 71% versus 79% and 30% versus 35% (Hollander, J. E. et al., J. Am. Coll. Cardiol. 34:95-105, 1999). P-selectin expression is greater in coronary atherectomy specimens from individuals with unstable angina than stable angina (Tenaglia, A. N. et al., Am. J. Cardiol. 79:742-747, 1997). Furthermore, plasma soluble P-selectin may be elevated to a greater degree in patients with acute myocardial infarction than in patients with unstable angina. Plasma soluble and membrane-bound P-selectin also is elevated in individuals with non-insulin dependent diabetes mellitus and congestive heart failure (Nomura, S. et al., Thromb. Haemost. 80:388-392, 1998; O'Connor, C. M. et al., Am. J. Cardiol. 83:1345-1349, 1999). Soluble P-selectin concentration is elevated in the plasma of individuals with idiopathic thrombocytopenic purpura, rheumatoid arthritis, hypercholesterolemia, acute stroke, atherosclerosis, hypertension, acute lung injury, connective tissue disease, thrombotic thrombocytopenic purpura, hemolytic uremic syndrome, disseminated intravascular coagulation, and chronic renal failure (Katayama, M. et al., Br. J. Haematol. 84:702-710, 1993; Haznedaroglu, I. C. et al., Acta Haematol. 101:16-20, 1999; Ertenli, I. et al., J. Rheumatol. 25:1054-1058, 1998; Davi, G. et al., Circulation 97:953-957, 1998; Frijns, C. J. et al., Stroke 28:2214-2218, 1997; Blann, A. D. et al., Thromb. Haemost. 77:1077-1080, 1997; Blann, A. D. et al., J. Hum. Hypertens. 11:607-609, 1997; Sakamaki, F. et al., A. J. Respir. Crit. Care Med. 151:1821-1826, 1995; Takeda, I. et al., Int. Arch. Allergy Immunol. 105:128-134, 1994; Chong, B. H. et al., Blood 83:1535-1541, 1994; Bonomini, M. et al., Nephron 79:399-407, 1998). Additionally, any condition that involves platelet activation can potentially be a source of plasma elevations in P-selectin. P-selectin is rapidly presented on the cell surface following platelet of endothelial cell activation. Soluble P-selectin that has been translated from an alternative mRNA lacking a transmembrane domain is also released into the extracellular space following this activation. Soluble P-selectin can also be formed by proteolysis involving membrane-bound P-selectin, either directly or indirectly. Plasma soluble P-selectin is elevated on admission in patients with acute myocardial infarction treated with tPA or coronary angioplasty, with a peak elevation occurring 4 hours after onset (Shimomura, H. et al., Am. J. Cardiol. 81:397-400, 1998). Plasma soluble P-selectin was elevated less than one hour following an anginal attack in patients with unstable angina, and the concentration decreased with time, approaching baseline more than 5 hours after attack onset (Ikeda, H. et al., Circulation 92:1693-1696, 1995). The plasma concentration of soluble P-selectin can approach 1 μg/ml in ACS (Ikeda, H. et al., Coron. Artery Dis. 5:515-518, 1994). Further investigation into the release of soluble P-selectin into and its removal from the bloodstream need to be conducted. P-selectin may be a sensitive and specific marker of platelet and endothelial cell activation, conditions that support thrombus formation and inflammation. It is not, however, a specific marker of ACS. When used with another marker that is specific for cardiac tissue injury, P-selectin may be useful in the discrimination of unstable angina and acute myocardial infarction from stable angina. Furthermore, soluble P-selectin may be elevated to a greater degree in acute myocardial infarction than in unstable angina. P-selectin normally exists in two forms, membrane-bound and soluble. Published investigations note that a soluble form of P-selectin is produced by platelets and endothelial cells, and by shedding of membrane-bound P-selectin, potentially through a proteolytic mechanism. Soluble P-selectin may prove to be the most useful currently identified marker of platelet activation, since its plasma concentration may not be as influenced by the blood sampling procedure as other markers of platelet activation, such as PF4 and β-TG.

Thrombin is a 37 kDa serine proteinase that proteolytically cleaves fibrinogen to form fibrin, which is ultimately integrated into a crosslinked network during clot formation. Antithrombin III (ATIII) is a 65 kDa serine proteinase inhibitor that is a physiological regulator of thrombin, factor XIa, factor XIIa, and factor IXa proteolytic activity. The inhibitory activity of ATIII is dependent upon the binding of heparin. Heparin enhances the inhibitory activity of ATIII by 2-3 orders of magnitude, resulting in almost instantaneous inactivation of proteinases inhibited by ATIII. ATIII inhibits its target proteinases through the formation of a covalent 1:1 stoichiometric complex. The normal plasma concentration of the approximately 100 kDa thrombin-ATIII complex (TAT) is <5 ng/ml (50 pM). TAT concentration is elevated in patients with acute myocardial infarction and unstable angina, especially during spontaneous ischemic episodes (Biasucci, L. M. et al., Am. J. Cardiol. 77:85-87, 1996; Kienast, J. et al., Thromb. Haemost. 70:550-553, 1993). Furthermore, TAT may be elevated in the plasma of individuals with stable angina (Manten, A. et al., Cardiovasc. Res. 40:389-395, 1998). Other published reports have found no significant differences in the concentration of TAT in the plasma of patients with ACS (Manten, A. et al., Cardiovasc. Res. 40:389-395, 1998; Hoffineister, H. M. et al., Atherosclerosis 144:151-157, 1999). Further investigation is needed to determine plasma TAT concentration changes associated with ACS. Elevation of the plasma TAT concentration is associated with any condition associated with coagulation activation, including stroke, surgery, trauma, disseminated intravascular coagulation, and thrombotic thrombocytopenic purpura. TAT is formed immediately following thrombin activation in the presence of heparin, which is the limiting factor in this interaction. TAT has a half-life of approximately 5 minutes in the bloodstream (Biasucci, L. M. et al., Am. J. Cardiol. 77:85-87, 1996). TAT concentration is elevated in, exhibits a sharp drop after 15 minutes, and returns to baseline less than 1 hour following coagulation activation. The plasma concentration of TAT can approach 50 ng/ml in ACS (Biasucci, L. M. et al., Circulation 93:2121-2127, 1996). TAT is a specific marker of coagulation activation, specifically, thrombin activation.

von Willebrand factor (vWF) is a plasma protein produced by platelets, megakaryocytes, and endothelial cells composed of 220 kDa monomers that associate to form a series of high molecular weight multimers. These multimers normally range in molecular weight from 600-20,000 kDa. vWF participates in the coagulation process by stabilizing circulating coagulation factor VIII and by mediating platelet adhesion to exposed subendothelium, as well as to other platelets. The A1 domain of vWF binds to the platelet glycoprotein Ib-IX-V complex and non-fibrillar collagen type VI, and the A3 domain binds fibrillar collagen types I and III (Emsley, J. et al., J. Biol. Chem. 273:10396-10401, 1998). Other domains present in the vWF molecule include the integrin binding domain, which mediates platelet-platelet interactions, the the protease cleavage domain, which appears to be relevant to the pathogenesis of type 11A von Willebrand disease. The interaction of vWF with platelets is tightly regulated to avoid interactions between vWF and platelets in normal physiologic conditions. vWF normally exists in a globular state, and it undergoes a conformation transition to an extended chain structure under conditions of high sheer stress, commonly found at sites of vascular injury. This conformational change exposes intramolecular domains of the molecule and allows vWF to interact with platelets. Furthermore, shear stress may cause vWF release from endothelial cells, making a larger number of vWF molecules available for interactions with platelets. The conformational change in vWF can be induced in vitro by the addition of non-physiological modulators like ristocetin and botrocetin (Miyata, S. et al., J. Biol. Chem. 271:9046-9053, 1996). At sites of vascular injury, vWF rapidly associates with collagen in the subendothelial matrix, and virtually irreversibly binds platelets, effectively forminig a bridge between platelets and the vascular subendothelium at the site of injury. Evidence also suggests that a conformational change in vWF may not be required for its interaction with the subendothelial matrix (Sixma, J. J. and de Groot, P. G., Mayo Clin. Proc. 66:628-633, 1991). This suggests that vWF may bind to the exposed subendothelial matrix at sites of vascular injury, undergo a conformational change because of the high localized shear stress, and rapidly bind circulating platelets, which will be integrated into the newly formed thrombus. Measurement of the total amount of vWF would allow one who is skilled in the art to identify changes in total vWF concentration associated with stroke or cardiovascular disease. This measurement could be performed through the measurement of various forms of the vWF molecule. Measurement of the A1 domain would allow the measurement of active vWF in the circulation, indicating that a pro-coagulant state exists because the A1 domain is accessible for platelet binding. In this regard, an assay that specifically measures vWF molecules with both the exposed A1 domain and either the integrin binding domain or the A3 domain would also allow for the identification of active vWF that would be available for mediating platelet-platelet interactions or mediate crosslinking of platelets to vascular subendothelium, respectively. Measurement of any of these vWF forms, when used in an assay that employs antibodies specific for the protease cleavage domain may allow assays to be used to determine the circulating concentration of various vWF forms in any individual, regardless of the presence of von Willebrand disease. The normal plasma concentration of vWF is 5-10 μg/ml, or 60-110% activity, as measured by platelet aggregation. The measurement of specific forms of vWF may be of importance in any type of vascular disease, including stroke and cardiovascular disease. The plasma vWF concentration is reportedly elevated in individuals with acute myocardial infarction and unstable angina, but not stable angina (Goto, S. et al., Circulation 99:608-613, 1999; Tousoulis, D.et al., Int. J. Cardiol. 56:259-262, 1996; Yazdani, S. et al., J Am Coll Cardiol 30:1284-1287, 1997; Montalescot, G. et al., Circulation 98:294-299). Furthermore, elevations of the plasma vWF concentration may be a predictor of adverse clinical outcome in patients with unstable angina (Montalescot, G. et al., Circulation 98:294-299). vWF concentrations also have been demonstrated to be elevated in patients with stroke and subarachnoid hemorrhage, and also appear to be useful in assessing risk of mortality following stroke (Blann, A. et al., Blood Coagul. Fibrinolysis 10:277-284, 1999; Hirashima, Y. et al. Neurochem Res. 22:1249-1255, 1997; Catto, A. J. et al., Thromb. Hemost. 77:1104-1108, 1997). The plasma concentration of vWF may be elevated in conjunction with any event that is associated with endothelial cell damage or platelet activation. vWF is present at high concentration in the bloodstream, and it is released from platelets and endothelial cells upon activation. vWF would likely have the greatest utility as a marker of platelet activation or, specifically, conditions that favor platelet activation and adhesion to sites of vascular injury. The conformation of VWF is also known to be altered by high shear stress, as would be associated with a partially stenosed blood vessel. As the blood flows past a stenosed vessel, it is subjected to shear stress considerably higher than is encountered in the circulation of an undiseased individual.

Tissue factor (TF) is a 45 kDa cell surface protein expressed in brain, kidney, and heart, and in a transcriptionally regulated manner on perivascular cells and monocytes. TF forms a complex with factor VIIa in the presence of Ca²⁺ ions, and it is physiologically active when it is membrane bound. This complex proteolytically cleaves factor X to form factor Xa. It is normally sequestered from the bloodstream. Tissue factor can be detected in the bloodstream in a soluble form, bound to factor VIIa, or in a complex with factor VIIa, and tissue factor pathway inhibitor that can also include factor Xa. TF also is expressed on the surface of macrophages, which are commonly found in atherosclerotic plaques. The normal serum concentration of TF is <0.2 ng/ml (4.5 pM). The plasma TF concentration is elevated in patients with ischemic heart disease (Falciani, M. et al., Thromb. Haemost. 79:495-499, 1998). TF is elevated in patients with unstable angina and acute myocardial infarction, but not in patients with stable angina (Falciani, M. et al., Thromb. Haemost. 79:495-499, 1998; Suefuji, H. et al., Am. Heart J. 134:253-259, 1997; Misumi, K. et al., Am. J. Cardiol. 81:22-26, 1998). Furthermore, TF expression on macrophages and TF activity in atherosclerotic plaques is more common in unstable angina than stable angina (Soejima, H. et al., Circulation 99:2908-2913, 1999; Kaikita, K. et al., Arterioscler. Thromb. Vasc. Biol. 17:2232-2237, 1997; Ardissino, D. et al., Lancet 349:769-771, 1997). The differences in plasma TF concentration in stable versus unstable angina may not be of statistical significance. Elevations in the serum concentration of TF are associated with any condition that causes or is a result of coagulation activation through the extrinsic pathway. These conditions can include subarachnoid hemorrhage, disseminated intravascular coagulation, renal failure, vasculitis, and sickle cell disease (Hirashima, Y. et al., Stroke 28:1666-1670, 1997; Takahashi, H. et al., Am. J. Hematol. 46:333-337, 1994; Koyama, T. et al., Br. J. Haematol. 87:343-347, 1994). TF is released immediately when vascular injury is coupled with extravascular cell injury. TF levels in ischemic heart disease patients can exceed 800 pg/ml within 2 days of onset (Falciani, M. et al., Thromb. Haemost. 79:495-499, 1998. TF levels were decreased in the chronic phase of acute myocardial infarction, as compared with the chronic phase (Suefuji, H. et al., Am. Heart J. 134:253-259, 1997). TF is a specific marker for activation of the extrinsic coagulation pathway and the presence of a general hypercoagulable state. It may be a sensitive marker of vascular injury resulting from plaque rupture

The coagulation cascade can be activated through either the extrinsic or intrinsic pathways. These enzymatic pathways share one final common pathway. The first step of the common pathway involves the proteolytic cleavage of prothrombin by the factor Xa/factor Va prothrombinase complex to yield active thrombin. Thrombin is a serine proteinase that proteolytically cleaves fibrinogen. Thrombin first removes fibrinopeptide A from fibrinogen, yielding desAA fibrin monomer, which can form complexes with all other fibrinogen-derived proteins, including fibrin degradation products, fibrinogen degradation products, desAA fibrin, and fibrinogen. The desAA fibrin monomer is generically referred to as soluble fibrin, as it is the first product of fibrinogen cleavage, but it is not yet crosslinked via factor XIIIa into an insoluble fibrin clot. DesAA fibrin monomer also can undergo further proteolytic cleavage by thrombin to remove fibrinopeptide B, yielding desAABB fibrin monomer. This monomer can polymerize with other desAABB fibrin monomers to form soluble desAABB fibrin polymer, also referred to as soluble fibrin or thrombus precursor protein (TpP™). TpP™ is the immediate precursor to insoluble fibrin, which forms a “mesh-like” structure to provide structural rigidity to the newly formed thrombus. In this regard, measurement of TPP™ in plasma is a direct measurement of active clot formation. The normal plasma concentration of TPP™ is <6 ng/ml (Laurino, J. P. et al., Ann. Clin. Lab. Sci. 27:338-345, 1997). American Biogenetic Sciences has developed an assay for TPP™ (U.S. Pat. Nos. 5,453,359 and 5,843,690) and states that its TpP™ assay can assist in the early diagnosis of acute myocardial infarction, the ruling out of acute myocardial infarction in chest pain patients, and the identification of patients with unstable angina that will progress to acute myocardial infarction. Other studies have confirmed that TpP™ is elevated in patients with acute myocardial infarction, most often within 6 hours of onset (Laurino, J. P. et al., Ann. Clin. Lab. Sci. 27:338-345, 1997; Carville, D. G. et al., Clin. Chem. 42:1537-1541, 1996). The plasma concentration of TPP™ is also elevated in patients with unstable angina, but these elevations may be indicative of the severity of angina and the eventual progression to acute myocardial infarction (Laurino, J. P. et al., Ann. Clin. Lab. Sci. 27:338-345, 1997). The concentration of TpP™ in plasma will theoretically be elevated during any condition that causes or is a result of coagulation activation, including disseminated intravascular coagulation, deep venous thrombosis, congestive heart failure, surgery, cancer, gastroenteritis, and cocaine overdose (Laurino, J. P. et al., Ann. Clin. Lab. Sci. 27:338-345, 1997). TpP™ is released into the bloodstream immediately following thrombin activation. TpP™ likely has a short half-life in the bloodstream because it will be rapidly converted to insoluble fibrin at the site of clot formation. Plasma TPP™ concentrations peak within 3 hours of acute myocardial infarction onset, returning to normal after 12 hours from onset. The plasma concentration of TpP™ can exceed 30 ng/ml in CVD (Laurino, J. P. et al., Ann. Clin. Lab. Sci. 27:338-345, 1997). TPP™ is a sensitive and specific marker of coagulation activation. It has been demonstrated that TPP™ is useful in the diagnosis of acute myocardial infarction, but only when it is used in conjunction with a specific marker of cardiac tissue injury.

(iii) Markers Related to Atherosclerotic Plaque Rupture

The appearance of markers related to atherosclerotic plaque rupture may preceed specific markers of myocardial injury. Potential markers of atherosclerotic plaque rupture include human neutrophil elastase, inducible nitric oxide synthase, lysophosphatidic acid, malondialdehyde-modified low density lipoprotein, and various members of the matrix metalloproteinase (MMP) family, including MMP −1, −2, −3, and −9.

Human neutrophil elastase (HNE) is a 30 kDa serine proteinase that is normally contained within the azurophilic granules of neutrophils. HNE is released upon neutrophil activation, and its activity is regulated by circulating α₁-proteinase inhibitor. Activated neutrophils are commonly found in atherosclerotic plaques, and rupture of these plaques may result in the release of HNE. The plasma HNE concentration is usually measured by detecting HNE-α₁-PI complexes. The normal concentration of these complexes is 50 ng/ml, which indicates a normal concentration of approximately 25 ng/ml (0.8 nM) for HNE. HNE release also can be measured through the specific detection of fibrinopeptide Bβ₃₀₋₄₃, a specific HNE-derived fibrinopeptide, in plasma. Plasma HNE is elevated in patients with coronary stenosis, and its elevation is greater in patients with complex plaques than those with simple plaques (Kosar, F. et al., Angiology 49:193-201, 1998; Amaro, A. et al., Eur. Heart J. 16:615-622, 1995). Plasma HNE is not significantly elevated in patients with stable angina, but is elevated inpatients with unstable angina and acute myocardial infarction, as determined by measuring fibrinopeptide Bβ₃₀₋₄₃, with concentrations in unstable angina being 2.5-fold higher than those associated with acute myocardial infarction (Dinerman, J. L. et al., J. Am. Coll. Cardiol. 15:1559-1563, 1990; Mehta, J. et al., Circulation 79:549-556, 1989). Serum NE is elevated in cardiac surgery, exercise-induced muscle damage, giant cell arteritis, acute respiratory distress syndrome, appendicitis, pancreatitis, sepsis, smoking-associated emphysema, and cystic fibrosis (Genereau, T. et al., J. Rheumatol. 25:710-713, 1998; Mooser, V. et al., Arterioscler. Thromb. Vasc. Biol. 19:1060-1065, 1999; Gleeson, M. et al. Eur. J. Appl. Physiol. 77:543-546, 1998; Gando, S. et al., J. Trauma 42:1068-1072, 1997; Eriksson, S. et al., Eur. J. Surg. 161:901-905, 1995; Liras, G. et al., Rev. Esp. Enferm. Dig. 87:641-652, 1995; Endo, S. et al., J. Inflamm. 45:136-142, 1995; Janoff, A., Annu Rev Med 36:207-216, 1985). HNE may also be released during blood coagulation (Plow, E. F. and Plescia, J., Thromb. Haemost. 59:360-363, 1988; Plow, E. F., J. Clin. Invest. 69:564-572, 1982). Serum elevations of HNE could also be associated with any non-specific infection or inflammatory state that involves neutrophil recruitment and activation. It is most likely released upon plaque rupture, since activated neutrophils are present in atherosclerotic plaques. HNE is presumably cleared by the liver after it has formed a complex with α₁-PI.

Inducible nitric oxide synthase (iNOS) is a 130 kDa cytosolic protein in epithelial cells macrophages whose expression is regulated by cytokines, including interferon-γ, interleukin-1β, interleukin-6, and tumor necrosis factor α, and lipopolysaccharide. iNOS catalyzes the synthesis of nitric oxide (NO) from L-arginine, and its induction results in a sustained high-output production of NO, which has antimicrobial activity and is a mediator of a variety of physiological and inflammatory events. NO production by iNOS is approximately 100 fold more than the amount produced by constitutively-expressed NOS (Depre, C. et al., Cardiovasc. Res. 41:465-472, 1999). There are no published investigations of plasma iNOS concentration changes associated with ACS. iNOS is expressed in coronary atherosclerotic plaque, and it may interfere with plaque stability through the production of peroxynitrate, which is a product of NO and superoxide and enhances platelet adhesion and aggregation (Depre, C. et al., Cardiovasc. Res. 41:465-472, 1999). iNOS expression during cardiac ischemia may not be elevated, suggesting that iNOS may be useful in the differentiation of angina from acute myocardial infarction (Hammerman, S. I. et al., Am. J. Physiol. 277:H1579-H1592, 1999; Kaye, D. M. et al., Life Sci 62:883-887, 1998). Elevations in the plasma iNOS concentration may be associated with cirrhosis, iron-deficiency anemia, or any other condition that results in macrophage activation, including bacterial infection (Jimenez, W. et al., Hepatology 30:670-676, 1999; Ni, Z. et al., Kidney Int. 52:195-201, 1997). iNOS may be released into the bloodstream as a result of atherosclerotic plaque rupture, and the presence of increased amounts of iNOS in the bloodstream may not only indicate that plaque rupture has occurred, but also that an ideal environment has been created to promote platelet adhesion. However, iNOS is not specific for atherosclerotic plaque rupture, and its expression can be induced during non-specific inflammatory conditions.

Lysophosphatidic acid (LPA) is a lysophospholipid intermediate formed in the synthesis of phosphoglycerides and triacylglycerols. It is formed by the acylation of glycerol-3 phosphate by acyl-coenzyme A and during mild oxidation of low-density lipoprotein (LDL). LPA is a lipid second messanger with vasoactive properties, and it can function as a platelet activator. LPA is a component of atherosclerotic lesions, particularly in the core, which is most prone to rupture (Siess, W., Proc. Natl. Acad. Sci. U.S. A. 96, 6931-6936, 1999). The normal plasma LPA concentration is 540 nM. Serum LPA is elevated in renal failure and in ovarian cancer and other gynecologic cancers (Sasagawa, T. et al., J. Nutr. Sci. Vitaminol. (Tokyo) 44:809-818, 1998; Xu, Y. et al., JAMA 280:719-723, 1998). In the context of unstable angina, LPA is most likely released as a direct result of plaque rupture. The plasma LPA concentration can exceed 60 μM in patients with gynecologic cancers (Xu, Y. et al., JAMA 280:719-723, 1998). Serum LPA may be a useful marker of atherosclerotic plaque rupture.

Malondialdehyde-modified low-density lipoprotein (MDA-modified LDL) is formed during the oxidation of the apoB-100 moiety of LDL as a result of phospholipase activity, prostaglandin synthesis, or platelet activation. MDA-modified LDL can be distinguished from oxidized LDL because MDA modifications of LDL occur in the absence of lipid peroxidation (Holvoet, P., Acta Cardiol. 53:253-260, 1998). The normal plasma concentration of MDA-modified LDL is less than 4 μg/ml (˜10 μM). Plasma concentrations of oxidized LDL are elevated in stable angina, unstable angina, and acute myocardial infarction, indicating that it may be a marker of atherosclerosis (Holvoet, P., Acta Cardiol. 53:253-260, 1998; Holvoct, P. et al., Circulation 98:1487-1494, 1998). Plasma MDA-modified LDL is not elevated in stable angina, but is significantly elevated in unstable angina and acute myocardial infarction (Holvoet, P., Acta Cardiol. 53:253-260, 1998; Holvoet, P. et al., Circulation 98:1487-1494, 1998; Holvoet, P. et al., JAMA 281:1718-1721, 1999). Plasma MDA-modified LDL is elevated in individuals with beta-thallasemia and in renal transplant patients (Livrea, M. A. et al., Blood 92:3936-3942, 1998; Ghanem, H. et al., Kidney Int. 49:488-493, 1996; van den Dorpel, M. A. et al., Transpl. Int. 9 Suppl. 1:S54-S57, 1996). Furthermore, serum MDA-modified LDL may be elevated during hypoxia (Balagopalakrishna, C. et al., Adv. Exp. Med. Biol. 411:337-345, 1997). The plasma concentration of MDA-modified LDL is elevated within 6-8 hours from the onset of chest pain. Plasma concentrations of MDA-modified LDL can approach 20 μg/ml (˜50 μM) in patients with acute myocardial infarction, and 15 μg/ml (˜40 μM) in patients with unstable angina (Holvoet, P. et al., Circulation 98:1487-1494, 1998). Plasma MDA-modified LDL has a half-life of less than 5 minutes in mice (Ling, W. et al., J. Clin. Invest. 100:244-252, 1997). MDA-modified LDL appears to be a specific marker of atherosclerotic plaque rupture in acute coronary symptoms. It is unclear, however, if elevations in the plasma concentration of MDA-modified LDL are a result of plaque rupture or platelet activation. The most reasonable explanation is that the presence of increased amounts of MDA-modified LDL is an indication of both events. MDA-modified LDL may be useful in discriminating unstable angina and acute myocardial infarction from stable angina.

Matrix metalloproteinase-1 (MMP-1), also called collagenase-1, is a 41/44 kDa zinc- and calcium-binding proteinase that cleaves primarily type I collagen, but can also cleave collagen types II, III, VII and X. The active 41/44 kDa enzyme can undergo autolysis to the still active 22/27 kDa form. MMP-1 is synthesized by a variety of cells, including smooth muscle cells, mast cells, macrophage-derived foam cells, T lymphocytes, and endothelial cells (Johnson, J. L. et al., Arterioscler. Thromb. Vasc. Biol. 18:1707-1715, 1998). MMP-1, like other MMPs, is involved in extracellular matrix remodeling, which can occur following injury or during intervascular cell migration. MMP-1 can be found in the bloodstream either in a free form or in complex with TIMP-1, its natural inhibitor. MMP-1 is normally found at a concentration of <25 ng/ml in plasma. MMP-1 is found in the shoulder region of atherosclerotic plaques, which is the region most prone to rupture, and may be involved in atherosclerotic plaque destabilization (Johnson, J. L. et al., Arterioscler. Thromb. Vasc. Biol. 18:1707-1715, 1998). Furthermore, MMP-1 has been implicated in the pathogenesis of myocardial reperfusion injury (Shibata, M. et al., Angiology 50:573-582, 1999). Serum MMP-1 maybe elevated inflammatory conditions that induce mast cell degranulation. Serum MMP-1 concentrations are elevated in patients with arthritis and systemic lupus erythematosus (Keyszer, G. et al., Z Rheumatol 57:392-398, 1998; Keyszer, G. J. Rheumatol. 26:251-258, 1999). Serum MMP-1 also is elevated in patients with prostate cancer, and the degree of elevation corresponds to the metastatic potential of the tumor (Baker, T. et al., Br. J. Cancer 70:506-512, 1994). The serum concentration of MMP-1 may also be elevated in patients with other types of cancer. Serum MMP-1 is decreased in patients with hemochromatosis and also in patients with chronic viral hepatitis, where the concentration is inversely related to the severity (George, D. K. et al., Gut 42:715-720, 1998; Murawaki, Y. et al., J. Gastroenterol. Hepatol. 14:138-145, 1999). Serum MMP-1 was decreased in the first four days following acute myocardial infarction, and increased thereafter, reaching peak levels 2 weeks after the onset of acute myocardial infarction (George, D. K. et al., Gut 42:715-720, 1998).

Matrix metalloproteinase-2 (MMP-2), also called gelatinase A, is a 66 kDa zinc- and calcium-binding proteinase that is synthesized as an inactive 72 kDa precursor. Mature MMP-3 cleaves type I gelatin and collagen of types IV, V, VII, and X. MMP-2 is synthesized by a variety of cells, including vascular smooth muscle cells, mast cells, macrophage-derived foam cells, T lymphocytes, and endothelial cells (Johnson, J. L. et al., Arterioscler. Thromb. Vasc. Biol. 18:1707-1715, 1998). MMP-2 is usually found in plasma in complex with TIMP-2, its physiological regulator (Murawaki, Y. et al., J. Hepatol. 30:1090-1098, 1999). The normal plasma concentration of MMP-2 is <˜550 ng/ml (8 nM). MMP-2 expression is elevated in vascular smooth muscle cells within atherosclerotic lesions, and it may be released into the bloodstream in cases of plaque instability (Kai, H. et al., J. Am. Coll. Cardiol. 32:368-372, 1998). Furthermore, MMP-2 has been implicated as a contributor to plaque instability and rupture (Shah, P. K. et al., Circulation 92:1565-1569, 1995). Serum MMP-2 concentrations were elevated in patients with stable angina, unstable angina, and acute myocardial infarction, with elevations being significantly greater in unstable angina and acute myocardial infarction than in stable angina (Kai, H. et al., J. Am. Coll. Cardiol. 32:368-372, 1998). There was no change in the serum MMP-2 concentration in individuals with stable angina following a treadmill exercise test (Kai, H. et al., J. Am. Coll. Cardiol. 32:368-372, 1998). Serum and plasma MMP-2 is elevated in patients with gastric cancer, hepatocellular carcinoma, liver cirrhosis, urothelial carcinoma, rheumatoid arthritis, and lung cancer (Murawaki, Y. et al., J. Hepatol. 30:1090-1098, 1999; Endo, K. et al., Anticancer Res. 17:2253-2258, 1997; Gohji, K. et al., Cancer 78:2379-2387, 1996; Gruber, B. L. et al., Clin. Immunol. Immunopathol. 78:161-171, 1996; Garbisa, S. et al., Cancer Res. 52:4548-4549, 1992). Furthermore, MMP-2 may also be translocated from the platelet cytosol to the extracellular space during platelet aggregation (Sawicki, G. et al., Thromb. Haemost. 80:836-839, 1998). MMP-2 was elevated on admission in the serum of individuals with unstable angina and acute myocardial infarction, with maximum levels approaching 1.5 μg/ml (25 nM) (Kai, H. et al., J. Am. Coll. Cardiol. 32:368-372, 1998). The serum MMP-2 concentration peaked 1-3 days after onset in both unstable angina and acute myocardial infarction, and started to return to normal after 1 week (Kai, H. et al., J. Am. Coil. Cardiol. 32:368-372, 1998).

Matrix metalloproteinase-3 (MMP-3), also called stromelysin-1, is a 45 kDa zinc- and calcium-binding proteinase that is synthesized as an inactive 60 kDa precursor. Mature MMP-3 cleaves proteoglycan, fibrinectin, laminin, and type IV collagen, but not type I collagen. MMP-3 is synthesized by a variety of cells, including smooth muscle cells, mast cells, macrophage-derived foam cells, T lymphocytes, and endothelial cells (Johnson, J. L. et al., Arterioscler. Thromb. Vasc. Biol. 18:1707-1715, 1998). MMP-3, like other MMPs, is involved in extracellular matrix remodeling, which can occur following injury or during intervascular cell migration. MMP-3 is normally found at a concentration of <125 ng/ml in plasma. The serum MMP-3 concentration also has been shown to increase with age, and the concentration in males is approximately 2 times higher in males than in females (Manicourt, D. H. et al., Arthritis Rheum. 37:1774-1783, 1994). MMP-3 is found in the shoulder region of atherosclerotic plaques, which is the region most prone to rupture, and may be involved in atherosclerotic plaque destabilization (Johnson, J. L. et al., Arterioscler. Thromb. Vasc. Biol. 18:1707-1715, 1998). Therefore, MMP-3 concentration may be elevated as a result of atherosclerotic plaque rupture in unstable angina. Serum MMP-3 may be elevated inflammatory conditions that induce mast cell degranulation. Serum MMP-3 concentrations are elevated in patients with arthritis and systemic lupus erythematosus (Zucker, S. et al. J. Rheumatol. 26:78-80, 1999; Keyszer, G. et al., Z Rheumatol. 57:392-398, 1998; Keyszer, G. et al. J. Rheumatol. 26:251-258, 1999). Serum MMP-3 also is elevated in patients with prostate and urothelial cancer, and also glomerulonephritis (Lein, M. et al., Urologe A 37:377-381, 1998; Gohji, K. et al., Cancer 78:2379-2387, 1996; Akiyama, K. et al., Res. Commun. Mol. Pathol. Pharmacol. 95:115-128, 1997). The serum concentration of MMP-3 may also be elevated in patients with other types of cancer. Serum MMP-3 is decreased in patients with hemochromatosis (George, D. K. et al., Gut 42:715-720, 1998).

Matrix metalloproteinase-9 (MMP-9) also called gelatinase B, is an 84 kDa zinc- and calcium-binding proteinase that is synthesized as an inactive 92 kDa precursor. Mature MMP-9 cleaves gelatin types I and V, and collagen types IV and V. MMP-9 exists as a monomer, a homodimer, and a heterodimer with a 25 kDa α₂-microglobulin-related protein (Triebel, S. et al., FEBS Lett. 314:386-388, 1992). MMP-9 is synthesized by a variety of cell types, most notably by neutrophils. The normal plasma concentration of MMP-9 is <35 ng/ml (400 pM). MMP-9 expression is elevated in vascular smooth muscle cells within atherosclerotic lesions, and it may be released into the bloodstream in cases of plaque instability (Kai, H. et al., J. Am. Coll. Cardiol. 32:368-372, 1998). Furthermore, MMP-9 may have a pathogenic role in the development of ACS (Brown, D. L. et al., Circulation 91:2125-2131, 1995). Plasma MMP-9 concentrations are significantly elevated in patients with unstable angina and acute myocardial infarction, but not stable angina (Kai, H. et al., J. Am. Coll. Cardiol. 32:368-372, 1998). The elevations in patients with acute myocardial infarction may also indicate that those individuals were suffering from unstable angina. Elevations in the plasma concentration of MMP-9 may also be greater in unstable angina than in acute myocardial infarction. There was no significant change in plasma MMP-9 levels after a treadmill exercise test in patients with stable angina (Kai, H. et al., J. Am. Coll . Cardiol. 32:368-372, 1998). Plasma MMP-9 is elevated in individuals with rheumatoid arthritis, septic shock, giant cell arteritis and various carcinomas (Gruber, B. L. et al., Clin. Immunol. Immunopathol. 78:161-171, 1996; Nakamura, T. et al., Am. J. Med. Sci. 316:355-360, 1998; Blankaert, D. et al., J. Acquir. Immune Defic. Syndr. Hum. Retrovirol. 18:203-209, 1998; Endo, K. et al. Anticancer Res. 17:2253-2258, 1997; Hayasaka, A. et al., Hepatology 24:1058-1062, 1996; Moore, D. H. et al., Gynecol. Oncol. 65:78-82, 1997; Sorbi, D. et al., Arthritis Rheum. 39:1747-1753, 1996; lizasa, T. et al., Clin., Cancer Res. 5:149-153, 1999). Furthermore, the plasma MMP-9 concentration may be elevated in stroke and cerebral hemorrhage (Mun-Bryce, S. and Rosenberg, G. A., J. Cereb. Blood Flow Metab. 18:1163-1172, 1998; Romanic, A. M. et al., Stroke 29:1020-1030, 1998; Rosenberg, G. A., J. Neurotrauma 12:833-842, 1995). MMP-9 was elevated on admission in the serum of individuals with unstable angina and acute myocardial infarction, with maximum levels approaching 150 ng/ml (1.7 nM) (Kai, H. et al., J. Am. Coll. Cardiol. 32:368-372, 1998). The serum MMP-9 concentration was highest on admission in patients unstable angina, and the concentration decreased gradually after treatment, approaching baseline more than 1 week after onset (Kai, H. et al., J. Am. Coll. Cardiol. 32:368-372, 1998).

(iv) Markers Related to Tissue Injury and Inflammation

C-reactive protein is a (CRP) is a homopentameric Ca²⁺-binding acute phase protein with 21 kDa subunits that is involved in host defense. CRP preferentially binds to phosphorylcholine, a common constituent of microbial membranes. Phosphorylcholine is also found in mammalian cell membranes, but it is not present in a form that is reactive with CRP. The interaction of CRP with phosphorylcholine promotes agglutination and opsonization of bacteria, as well as activation of the complement cascade, all of which are involved in bacterial clearance. Furthermore, CRP can interact with DNA and histones, and it has been suggested that CRP is a scavenger of nuclear material released from damaged cells into the circulation (Robey, F. A. et al., J. Biol. Chem. 259:7311-7316, 1984). CRP synthesis is induced by I1-6, and indirectly by IL-1, since IL-1 can trigger the synthesis of IL-6 by Kupffer cells in the hepatic sinusoids. The normal plasma concentration of CRP is <3 μg/ml (30 nM) in 90% of the healthy population, and <101 g/ml (100 nM) in 99% of healthy individuals. Plasma CRP concentrations can be measured by rate nephelometry or ELISA. The plasma concentration of CRP is significantly elevated in patients with acute myocardial infarction and unstable angina, but not stable angina (Biasucci, L. M. et al., Circulation 94:874-877, 1996; Biasucci, L. M. et al., Am. J. Cardiol. 77:85-87, 1996; Benamer, H. et al., Am. J. Cardiol. 82:845-850, 1998; Caligiuri, G. et al., J. Am. Coll. Cardiol. 32:1295-1304, 1998; Curzen, N. P. et al., Heart 80:23-27, 1998; Dangas, G. et al., Am. J. Cardiol. 83:583-5, A7, 1999). CRP may also be elevated in the plasma of individuals with variant or resolving unstable angina, but mixed results have been reported (Benamer, H. et al., Am. J. Cardiol. 82:845-850, 1998; Caligiuri, G. et al., J. Am. Coil. Cardiol. 32:1295-1304, 1998). The concentration of CRP will be elevated in the plasma from individuals with any condition that may elicit an acute phase response, such as infection, surgery, trauma, and stroke. CRP is a secreted protein that is released into the bloodstream soon after synthesis. CRP synthesis is upregulated by IL-6, and the plasma CRP concentration is significantly elevated within 6 hours of stimulation (Biasucci, L. M. et al., Am. J. Cardiol. 77:85-87, 1996). The plasma CRP concentration peaks approximately 50 hours after stimulation, and begins to decrease with a half-life of approximately 19 hours in the bloodstream (Biasucci, L. M. et al., Am. J. Cardiol. 77:85-87, 1996). Other investigations have confirmed that the plasma CRP concentration in individuals with unstable angina (Biasucci, L. M. et al., Circulation 94:874-877, 1996). The plasma concentration of CRP can approach 100 μg/ml (1 μM) in individuals with ACS (Biasucci, L. M. et al., Circulation 94:874-877, 1996; Liuzzo, G. et al., Circulation 94:2373-2380, 1996). CRP is a specific marker of the acute phase response. Elevations of CRP have been identified in the plasma of individuals with acute myocardial infarction and unstable angina, most likely as a result of activation of the acute phase response associated with atherosclerotic plaque rupture or cardiac tissue injury.

Interleukin-1β (IL-1β) is a 17 kDa secreted proinflammatory cytokine that is involved in the acute phase response and is a pathogenic mediator of many diseases. IL-1β is normally produced by macrophages and epithelial cells. IL-1β is also released from cells undergoing apoptosis. The normal serum concentration of IL-1β is <30 pg/ml (1.8 pM). In theory, IL-1β would be elevated earlier than other acute phase proteins such as CRP in unstable angina and acute myocardial infarction, since IL-1β is an early participant in the acute phase response. Furthermore, IL-1β is released from cells undergoing apoptosis, which may be activated in the early stages of ischemia. In this regard, elevation of the plasma IL-1β concentration associated with ACS requires further investigation using a high-sensitivity assay. Elevations of the plasma IL-1β concentration are associated with activation of the acute phase response in proinflammatory conditions such as trauma and infection. IL-1β has a biphasic physiological half-life of 5 minutes followed by 4 hours (Kudo, S. et al., Cancer Res. 50:5751-5755, 1990). IL-1β is released into the extracellular milieu upon activation of the inflammatory response or apoptosis.

Interleukin-1 receptor antagonist (IL-1ra) is a 17 kDa member of the IL-1 family predominantly expressed in hepatocytes, epithelial cells, monocytes, macrophages, and neutrophils. IL-1ra has both intracellular and extracellular forms produced through alternative splicing. IL-1ra is thought to participate in the regulation of physiological IL-1 activity. IL-1ra has no IL-1-like physiological activity, but is able to bind the IL-1 receptor on T-cells and fibroblasts with an affinity similar to that of IL-1β, blocking the binding of IL-1α and IL-1β and inhibiting their bioactivity (Stockman, B. J. et al., Biochemistry 31:5237-5245, 1992; Eisenberg, S. P. et al., Proc. Natl. Acad. Sci. U.S. A. 88:5232-5236, 1991; Carter, D. B. et al., Nature 344:633-638, 1990). IL-1ra is normally present in higher concentrations than IL-1 in plasma, and it has been suggested that IL-1ra levels are a better correlate of disease severity than IL-1 (Biasucci, L. M. et al., Circulation 99:2079-2084, 1999). Furthermore, there is evidence that IL-1ra is an acute phase protein (Gabay, C. et al., J. Clin. Invest. 99:2930-2940, 1997). The normal plasma concentration of IL-1ra is <200 pg/ml (12 pM). The plasma concentration of IL-1ra is elevated in patients with acute myocardial infarction and unstable angina that proceeded to acute myocardial infarction, death, or refractory angina (Biasucci, L. M. et al., Circulation 99:2079-2084, 1999; Latini, R. et al., J. Cardiovasc. Pharmacol. 23:1-6, 1994). Furthermore, IL-1ra was significantly elevated in severe acute myocardial infarction as compared to uncomplicated acute myocardial infarction (Latini, R. et al., J. Cardiovasc. Pharmacol. 23:1-6, 1994). Elevations in the plasma concentration of IL-1ra are associated with any condition that involves activation of the inflammatory or acute phase response, including infection, trauma, and arthritis. IL-1ra is released into the bloodstream in pro-inflammatory conditions, and it may also be released as a participant in the acute phase response. The major sources of clearance of IL-1ra from the bloodstream appear to be kidney and liver (Kim, D. C. et al., J. Pharm. Sci. 84:575-580, 1995). IL-1ra concentrations were elevated in the plasma of individuals with unstable angina within 24 hours of onset, and these elevations may even be evident within 2 hours of onset (Biasucci, L. M. et al., Circulation 99:2079-2084, 1999). In patients with severe progression of unstable angina, the plasma concentration of IL-1ra was higher 48 hours after onset than levels at admission, while the concentration decreased in patients with uneventful progression (Biasucci, L. M. et al., Circulation 99:2079-2084, 1999). In addition, the plasma concentration of IL-1ra associated with unstable angina can approach 1.4 ng/ml (80 pM). Changes in the plasma concentration of IL-1ra appear to be related to disease severity. Furthermore, it is likely released in conjunction with or soon after IL-1 release in pro-inflammatory conditions, and it is found at higher concentrations than IL-1. This indicates that IL-1ra may be a useful indirect marker of IL-i activity, which elicits the production of IL-6.

Interleukin-6 (IL-6) is a 20 kDa secreted protein that is a hematopoietin family proinflammatory cytokine. IL-6 is an acute-phase reactant and stimulates the synthesis of a variety of proteins, including adhesion molecules. Its major function is to mediate the acute phase production of hepatic proteins, and its synthesis is induced by the cytokine IL-1. IL-6 is normally produced by macrophages and T lymphocytes. The normal serum concentration of IL-6 is <3 pg/ml (0.15 pM). The plasma concentration of IL-6 is elevated in patients with acute myocardial infarction and unstable angina, to a greater degree in acute myocardial infarction (Biasucci, L. M. et al., Circulation 94:874-877, 1996; Manten, A. et al., Cardiovasc. Res. 40:389-395, 1998; Biasucci, L. M. et al., Circulation 99:2079-2084, 1999). IL-6 is not significantly elevated in the plasma of patients with stable angina (Biasucci, L. M. et al., Circulation 94:874-877, 1996; Manten, A. et al., Cardiovasc. Res. 40:389-395, 1998). Furthermore, IL-6 concentrations increase over 48 hours from onset in the plasma of patients with unstable angina with severe progression, but decrease in those with uneventful progression (Biasucci, L. M. et al., Circulation 99:2079-2084, 1999). This indicates that IL-6 may be a useful indicator of disease progression. Plasma elevations of IL-6 are associated with any nonspecific proinflammatory condition such as trauma, infection, or other diseases that elicit an acute phase response. IL-6 has a half-life of 4.2 hours in the bloodstream and is elevated following acute myocardial infarction and unstable angina (Manten, A. et al., Cardiovasc. Res. 40:389-395, 1998). The plasma concentration of IL-6 is elevated within 8-12 hours of acute myocardial infarction onset, and can approach 100 pg/ml. The plasma concentration of IL-6 in patients with unstable angina was elevated at peak levels 72 hours after onset, possibly due to the severity of insult (Biasucci, L. M. et al., Circulation 94:874-877, 1996).

Tumor necrosis factor α (TNFαc) is a 17 kDa secreted proinflammatory cytokine that is involved in the acute phase response and is a pathogenic mediator of many diseases. TNFα is normally produced by macrophages and natural killer cells. TNF-alpha is a protein of 185 amino acids glycosylated at positions 73 and 172. It is synthesized as a precursor protein of 212 amino acids. Monocytes express at least five different molecular forms of TNF-alpha with molecular masses of 21.5-28 kDa. They mainly differ by post-translational alterations such as glycosylation and phosphorylation. The normal serum concentration of TNFαc is <40 pg/ml (2 pM). The plasma concentration of TNFα is elevated in patients with acute myocardial infarction, and is marginally elevated in patients with unstable angina (Li, D. et al., Am. Heart J. 137:1145-1152, 1999; Squadrito, F. et al., Inflamm. Res. 45:14-19, 1996; Latini, R. et al., J. Cardiovasc. Pharmacol. 23:1-6, 1994; Carlstedt, F. et al., J. Intern. Med. 242:361-365, 1997). Elevations in the plasma concentration of TNFα are associated with any proinflammatory condition, including trauma, stroke, and infection. TNFα has a half-life of approximately 1 hour in the bloodstream, indicating that it may be removed from the circulation soon after symptom onset. In patients with acute myocardial infarction, TNFα was elevated 4 hours after the onset of chest pain, and gradually declined to normal levels within 48 hours of onset (Li, D. et al., Am. Heart J. 137:1145-1152, 1999). The concentration of TNFα in the plasma of acute myocardial infarction patients exceeded 300 pg/ml (15 pM) (Squadrito, F. et al., Inflamm. Res. 45:14-19, 1996). Release of TNFα by monocytes has also been related to the progression of pneumoconiosis in caol workers. Schins and Borm, Occup. Environ. Med. 52: 441-50 (1995).

Soluble intercellular adhesion molecule (sICAM-1), also called CD54, is a 85-110 kDa cell surface-bound immunoglobulin-like integrin ligand that facilitates binding of leukocytes to antigen-presenting cells and endothelial cells during leukocyte recruitment and migration. sICAM-1 is normally produced by vascular endothelium, hematopoietic stem cells and non-hematopoietic stem cells, which can be found in intestine and epidermis. sICAM-1 can be released from the cell surface during cell death or as a result of proteolytic activity. The normal plasma concentration of sICAM-1 is approximately 250 ng/ml (2.9 nM). The plasma concentration of sICAM-1 is significantly elevated in patients with acute myocardial infarction and unstable angina, but not stable angina (Pellegatta, F. et al., J. Cardiovasc. Pharmacol. 30:455-460, 1997; Miwa, K. et al., Cardiovasc. Res. 36:37-44, 1997; Ghaisas, N. K. et al., Am. J. Cardiol. 80:617-619, 1997; Ogawa, H. et al, Am. J. Cardiol. 83:38-42, 1999). Furthermore, ICAM-1 is expressed in atherosclerotic lesions and in areas predisposed to lesion formation, so it may be released into the bloodstream upon plaque rupture (Iiyama, K. et al., Circ. Res. 85:199-207, 1999; Tenaglia, A. N. et al., Am. J. Cardiol. 79:742-747, 1997). Elevations of the plasma concentration of sICAM-1 are associated with ischemic stroke, head trauma, atherosclerosis, cancer, preeclampsia, multiple sclerosis, cystic fibrosis, and other nonspecific inflammatory states (Kim, J. S., J. Neurol. Sci. 137:69-78, 1996; Laskowitz, D. T. et al., J. Stroke Cerebrovasc. Dis. 7:234-241, 1998). The plasma concentration of sICAM-1 is elevated during the acute stage of acute myocardial infarction and unstable angina. The elevation of plasma sICAM-1 reaches its peak within 9-12 hours of acute myocardial infarction onset, and returns to normal levels within 24 hours (Pellegatta, F. et al, J. Cardiovasc. Pharmacol. 30:455-460, 1997). The plasma concentration of sICAM can approach 700 ng/ml (8 nM) in patients with acute myocardial infarction (Pellegatta, F. et al., J. Cardiovasc. Pharmacol. 30:455-460, 1997). sICAM-1 is elevated in the plasma of individuals with acute myocardial infarction and unstable angina, but it is not specific for these diseases. It may, however, be useful marker in the differentiation of acute myocardial infarction and unstable angina from stable angina since plasma elevations are not associated with stable angina. Interestingly, ICAM-1 is present in atherosclerotic plaques, and may be released into the bloodstream upon plaque rupture.

Vascular cell adhesion molecule (VCAM), also called CD106, is a 100-110 kDa cell surface-bound immunoglobulin-like integrin ligand that facilitates binding of B lymphocytes and developing T lymphocytes to antigen-presenting cells during lymphocyte recruitment. VCAM is normally produced by endothelial cells, which line blood and lymph vessels, the heart, and other body cavities. VCAM-1 can be released from the cell surface during cell death or as a result of proteolytic activity. The normal serum concentration of sVCAM is approximately 650 ng/ml (6.5 nM). The plasma concentration of sVCAM-1 is marginally elevated in patients with acute myocardial infarction, unstable angina, and stable angina (Mulvihill, N. et al., Am. J. Cardiol. 83:1265-7, A9, 1999; Ghaisas, N. K. et al., Am. J. Cardiol. 80:617-619, 1997). However, sVCAM-1 is expressed in atherosclerotic lesions and its plasma concentration may correlate with the extent of atherosclerosis (Iiyama, K. et al., Circ. Res. 85:199-207, 1999; Peter, K. et al., Arterioscler. Thromb. Vasc. Biol. 17:505-512, 1997). Elevations in the plasma concentration of sVCAM-1 are associated with ischemic stroke, cancer, diabetes, preeclampsia, vascular injury, and other nonspecific inflammatory states (Bitsch, A. et al., Stroke 29:2129-2135, 1998; Otsuki, M. et al., Diabetes 46:2096-2101, 1997; Banks, R. E. et al., Br. J. Cancer 68:122-124, 1993; Steiner, M. et al., Thromb. Haemost. 72:979-984, 1994; Austgulen, R. et al., Eur. J. Obstet. Gynecol. Reprod. Biol. 71:53-58, 1997).

Monocyte chemotactic protein-I (MCP-1) is a 10 kDa chemotactic factor that attracts monocytes and basophils, but not neutrophils or eosiniphils. MCP-1 is normally found in equilibrium between a monomeric and homodimeric form, and it is normally produced in and secreted by monocytes and vascular endothelial cells (Yoshimura, T. et al., FEBS Lett. 244:487-493, 1989; Li, Y. S. et al., Mol. Cell. Biochem. 126:61-68, 1993). MCP-1 has been implicated in the pathogenesis of a variety of diseases that involve monocyte infiltration, including psoriasis, rheumatoid arthritis, and atherosclerosis. The normal concentration of MCP-1 in plasma is <0.1 ng/ml. The plasma concentration of MCP-1 is elevated in patients with acute myocardial infarction, and may be elevated in the plasma of patients with unstable angina, but no elevations are associated with stable angina (Soejima, H. et al., J. Am. Coil. Cardiol. 34:983-988, 1999; Nishiyama, K. et al., Jpn. Circ. J. 62:710-712, 1998; Matsumori, A. et al., J. Mol. Cell. Cardiol. 29:419-423, 1997). Interestingly, MCP-1 also may be involved in the recruitment of monocytes into the arterial wall during atherosclerosis. Elevations of the serum concentration of MCP-1 are associated with various conditions associated with inflammation, including alcoholic liver disease, interstitial lung disease, sepsis, and systemic lupus erythematosus (Fisher, N. C. et al., Gut 45:416-420, 1999; Suga, M. et al., Eur. Respir. J. 14:376-382, 1999; Bossink, A. W. et al., Blood 86:3841-3847, 1995; Kaneko, H. et al. J. Rheumatol. 26:568-573, 1999). MCP-1 is released into the bloodstream upon activation of monocytes and endothelial cells. The concentration of MCP-1 in plasma form patients with acute myocardial infarction has been reported to approach 1 ng/ml (100 pM), and can remain elevated for one month (Soejima, H. et al., J. Am. Coll. Cardiol. 34:983-988, 1999). MCP-1 is a specific marker of the presence of a pro-inflammatory condition that involves monocyte migration.

Caspase-3, also called CPP-32, YAMA, and apopain, is an interleukin-1β converting enzyme (ICE)-like intracellular cysteine proteinase that is activated during cellular apoptosis. Caspase-3 is present as an inactive 32 kDa precursor that is proteolytically activated during apoptosis induction into a heterodimer of 20 kDa and 11 kDa subunits (Femandes-Alnemri, T. et al., J. Biol. Chem. 269:30761-30764, 1994). Its cellular substrates include poly(ADP-ribose) polymerase (PARP) and sterol regulatory element binding proteins (SREBPs) (Liu, X. et al., J. Biol. Chem. 271:13371-13376, 1996). The normal plasma concentration of caspase-3 is unknown. There are no published investigations into changes in the plasma concentration of caspase-3 associated with ACS. There are increasing amounts of evidence supporting the hypothesis of apoptosis induction in cardiac myocytes associated with ischemia and hypoxia (Saraste, A., Herz 24:189-195, 1999; Ohtsuka, T. et al., Coron. Artery Dis. 10:221-225, 1999; James, T. N., Coron. ArteryDis. 9:291-307, 1998; Bialik, S. et al., J. Clin. Invest. 100:1363-1372, 1997; Long, X. et al., J. Clin. Invest. 99:2635-2643, 1997). Elevations in the plasma caspase-3 concentration may be associated with any physiological event that involves apoptosis. There is evidence that suggests apoptosis is induced in skeletal muscle during and following exercise and in cerebral ischemia (Carraro, U. and Franceschi, C., Aging (Milano) 9:19-34, 1997; MacManus, J. P. et al., J. Cereb. Blood Flow Metab. 19:502-510, 1999).

Hemoglobin (Hb) is an oxygen-carrying iron-containing globular protein found in erythrocytes. It is a heterodimer of two globin subunits. α₂γ₂ is referred to as fetal Hb, α₂γ₂ is called adult HbA, and α₂δ₂ is called adult HbA₂. 90-95% of hemoglobin is HbA, and the α₂ globin chain is found in all Hb types, even sickle cell hemoglobin. Hb is responsible for carrying oxygen to cells throughout the body. Hbα₂ is not normally detected in serum.

Human lipocalin-type prostaglandin D synthase (hPDGS), also called β-trace, is a 30 kDa glycoprotein that catalyzes the formation of prostaglandin D2 from prostaglandin H. The upper limit of hPDGS concentrations in apparently healthy individuals is reported to be approximately 420 ng/ml (Patent No. EP0999447A1). Elevations of hPDGS have been identified in blood from patients with unstable angina and cerebral infarction (Patent No. EP0999447A1). Furthermore, hPDGS appears to be a useful marker of ischemic episodes, and concentrations of hPDGS were found to decrease over time in a patient with angina pectoris following percutaneous transluminal coronary angioplasty (PTCA), suggesting that the hPGDS concentration decreases as ischemia is resolved (Patent No. EP0999447A1).

Mast cell tryptase, also known as alpha tryptase, is a 275 amino acid (30.7 kDa) protein that is the major neutral protease present in mast cells. Mast cell tryptase is a specific marker for mast cell activation, and is a marker of allergic airway inflammation in asthma and in allergic reactions to a diverse set of allergens. See, e.g., Taira et al., J. Asthma 39: 315-22 (2002); Schwartz et al., N. Engl. J. Med. 316: 1622-26 (1987). Elevated serum tryptase levels (>1 ng/mL) between 1 and 6 hours after an event provides a specific indication of mast cell degranulation.

Eosinophil cationic protein (ECP) is a heterogeneous protein with molecular weight variants from 16-24 kDa and a pI of pH 10.8. ECP is highly cytotoxic and is released by activated cosinophils. Venge, Clinical and experimental allergy, 23 (suppl. 2): 3-7 (1993). Concentrations of ECP in the bronchoalveolar lavage fluid (BALF) of asthma patients vary with the severity of their disease, and ECP concentrations in sputum have also been shown to reflect the pathophysiology of the disease. Bousquet et al., New Engl. J. Med. 323: 1033-9 (1990). Virchow et al., Am. Rev. Respir. Dis. 146: 604-6 (1992). Assessment of serum ECP may be assumed to reflect pulmonary inflammation in bronchial asthma. Koller et al., Arch. Dis. Childhood 73: 413-7 (1995); see also, Sorkness et al., Clin. Exp. Allergy 32: 1355-59 (2002); Badr-elDin et al., East Mediterr. Health J. 5: 664-75 (1999).

KL-6 (also referred to as MUC1) is a high molecular weight (>300 kDa) mucinous glycoprotein expressed on pneumonocytes. Serum levels of KL-6 are reportedly elevated in interstitial lung diseases, which are characterized by exertional dyspnea. KL-6 has been shown to be a marker of various interstitial lung diseases, including pulmonary fibrosis, interstitial pneumonia, sarcoidosis, and interstitial pneumonitis. See, e.g., Kobayashi and Kitamura, Chest 108: 311-15 (1995); Kohno, J. Med. Invest. 46: 151-58 (1999); Bandoh et al., Ann. Rheum. Dis. 59: 257-62 (2000); and Yamane et al., J. Rheumatol. 27: 930-4 (2000).

Procalcitonin is a 116 amino acid (14.5 kDa) protein encoded by the Calc-1 gene located on chromosome 11p15.4. The Calc-1 gene produces two transcripts that are the result of alternative splicing events. Pre-procalcitonin contains a 25 amino acid signal peptide which is processed by C-cells in the thyrois to a 57 amino acid N-terminal fragment, a 32 amino acid calcitonin fragment, and a 21 amino acid katacalcin fragment. Procalcitonin is secreted intact as a glycosylated product by other body cells. Whicher et al., Ann. Clin. Biochem. 38: 483-93 (2001). Plasma procalcitonin has been identified as a marker of sepsis and its severity (Yukioka et al., Ann. Acad. Med. Singapore 30: 528-31 (2001)), with day 2 procalcitonin levels predictive of mortality (Pettila et al., Intensive Care Med. 28: 1220-25 (2002).

Interleukin 10 (“IL-10”) is a 160 amino acid (18.5 kDa predicted mass) cytokine that is a member of the four α-helix bundle family of cytokines. In solution, IL-10 forms a homodimer having an apparent molecular weight of 39 kDa. The human IL-10 gene is located on chromosome 1. Viera et al., Proc. Natl. Acad Sci. USA 88: 1172-76 (1991); Kim et al., J. Immunol. 148: 3618-23 (1992). Overproduction of IL-10 has been identified as a marker in sepsis, and is predictive of severity and mortality. Gogos et al., J. Infect. Dis. 181: 176-80 (2000).

Exemplary Marker Panels for Distinguishing Systolic and Diastolic Heart Failure

Congestive heart failure is a heterogenous condition arising from two primary pathologies: left ventricular diastolic dysfunction and systolic dysfunction, which occur either alone or in combination. Gaasch, JAMA 271: 1276-80 (1994). As many as 40 percent of patients with clinical heart failure have diastolic dysfunction with normal systolic function. Soufer et al., Am. J. Cardiol. 55: 1032-6 (1984). Patient care decisions and prognosis hinge upon determination of the presence of one or both of these pathologies. Shamsham and Mitchell, Am Fam Physician 2000;61:1319-28 (2000).

Recently, BNP has been reported as a useful marker in the diagnosis of congestive heart failure. Dao et al., J. Am. Coll. Cardiol. 37: 379-85 (2001). However, BNP levels alone are not able to distinguish diastolic dysfunction from systolic dysfunction. Krishnaswamy et al., Am. J. Med. 111: 274-79 (2001).

Assay Measurement Strategies

Numerous methods and devices are well known to the skilled artisan for the detection and analysis of the markers of the instant invention. With regard to polypeptides or proteins in patient test samples, immunoassay devices and methods are often used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety, including all tables, figures and claims. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule. See, e.g., U.S. Pat. No. 5,631,171; and 5,955,377, each of which is hereby incorporated by reference in its entirety, including all tables, figures and claims.

Preferably the markers are analyzed using an immunoassay, although other methods are well known to those skilled in the art (for example, the measurement of marker RNA levels). The presence or amount of a marker is generally determined using antibodies specific for each marker and detecting specific binding. Any suitable immunoassay may be utilized, for example, enzyme-linked immunoassays (ELISA), radioimmunoassays (RIAs), competitive binding assays, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies specific for the markers is also contemplated by the present invention. The antibodies could be immobilized onto a variety of solid supports, such as magnetic or chromatographic matrix particles, the surface of an assay place (such as microtiter wells), pieces of a solid substrate material (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a colored spot.

The analysis of a plurality of markers may be carried out separately or simultaneously with one test sample. Several markers may be combined into one test for efficient processing of a multiple of samples. In addition, one skilled in the art would recognize the value of testing multiple samples (for example, at successive time points) from the same individual. Such testing of serial samples will allow the identification of changes in marker levels over time. Increases or decreases in marker levels, as well as the absence of change in marker levels, would provide useful information about the disease status that includes, but is not limited to identifying the approximate time from onset of the event, the presence and amount of salvagable tissue, the appropriateness of drug therapies, the effectiveness of various therapies, identification of the severity of the event, identification of the disease severity, and identification of the patient's outcome, including risk of future events.

A panel consisting of the markers referenced above may be constructed to provide relevant information related to differential diagnosis. Such a panel may be constucted using 1, 2, 3, 4, 5, 6, 7, 8, 9, 10, 15, 20, or more or individual markers. The analysis of a single marker or subsets of markers comprising a larger panel of markers could be carried out by one skilled in the art to optimize clinical sensitivity or specificity in various clinical settings. These include, but are not limited to ambulatory, urgent care, critical care, intensive care, monitoring unit, inpatient, outpatient, physician office, medical clinic, and health screening settings. Furthermore, one skilled in the art can use a single marker or a subset of markers comprising a larger panel of markers in combination with an adjustment of the diagnostic threshold in each of the aforementioned settings to optimize clinical sensitivity and specificity. The clinical sensitivity of an assay is defined as the percentage of those with the disease that the assay correctly predicts, and the specificity of an assay is defined as the percentage of those without the disease that the assay correctly predicts (Tietz Textbook of Clinical Chemistry, 2^{nd} edition, Carl Burtis and Edward Ashwood eds., W. B. Saunders and Company, p. 496).

The analysis of markers could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings. Particularly useful physical formats comprise surfaces having a plurality of discrete, adressable locations for the detection of a plurality of different analytes. Such formats include protein microarrays, or “protein chips” (see, e.g., Ng and Ilag, J. Cell Mol. Med. 6: 329-340 (2002)) and certain capillary devices (see, e.g., U.S. Pat. No. 6,019,944)

In another embodiment, the present invention provides a kit for the analysis of markers. Such a kit preferably comprises devises and reagents for the analysis of at least one test sample and instructions for performing the assay. Optionally the kits may contain one or more means for using information obtained from immunoassays performed for a marker panel to rule in or out certain diagnoses.

Selecting a Treatment Regimen

Just as the potential causes of any particular nonspecific symptom may be a large and diverse set of conditions, the appropriate treatments for these potential causes may be equally large and diverse. However, once a diagnosis is obtained, the clinician can readily select a treatment regimen that is compatible with the diagnosis. Taking just some of the causes of dyspnea for example, initial treatment for pulmonary embolism is supportive, involving analgesics, oxygen, and potentially β-adrenergic stimulation. Thrombolytic therapy or embolectomy may be indicated. In contrast, treatment for systolic dysfunction in congestive heart failure can include therapeutic amounts of ACE inhibitors, digoxin, β-blockers, and diuretics. In particularly serious chronic heart failure, heart transplant may be indicated. The skilled artisan is aware of appropriate treatments for numerous diseases discussed in relation to the methods of diagnosis described herein. See, e.g., Merck Manual of Diagnosis and Therapy, 17^{th} Ed. Merck Research Laboratories, Whitehouse Station, N.J., 1999.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of”and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

TABLE 1

| | SENSE OF | CUTOFF | LENGTH | WEIGHTING |
|---|---|---|---|---|
| MARKER | MARKER | LOCATION | OF CUTOFF | COEFF. |
| Analyte 1 | Incr. | 18.01 | 0.90 | 0.67 |
| Analyte 2 | Incr. | 128.92 | 0.83 | 0.75 |
| Analyte 3 | Incr. | 86.17 | 1.00 | 0.73 |
| Analyte 4 | Incr. | 41.46 | 0.99 | 0.55 |
| Analyte 5 | Incr. | 228.23 | 1.00 | 0.74 |
| Analyte 6 | Incr. | 21.87 | 1.00 | 0.82 |
| Analyte 7 | Incr. | 2.63 | 0.96 | 0.66 |
| Analyte 8 | Decr. | 65.92 | 0.14 | 0.66 |
| Analyte 9 | Incr. | 582.80 | 0.82 | 0.57 |
| Analyte 10 | Incr. | 66.07 | 1.00 | 0.65 |
| Analyte 11 | Incr. | 0.00 | 1.00 | 0.81 |
| Analyte 12 | Incr. | 189.17 | 0.57 | 0.84 |
| Analyte 13 | Incr. | 122.76 | 1.00 | 0.68 |
| Analyte 14 | Incr. | 45.72 | 1.00 | 0.67 |
| Analyte 15 | Incr. | 1632.97 | 1.00 | 0.72 |
| Analyte 16 | Incr. | 48.93 | 0.74 | 0.82 |
| Analyte 17 | Incr. | 8352.03 | 0.18 | 0.85 |
| Analyte 18 | Incr. | 4528.32 | 0.18 | 0.78 |
| Analyte 19 | Incr. | 1424.02 | 0.56 | 0.83 |
| Analyte 20 | Incr. | 1827.05 | 0.49 | 0.84 |
| Analyte 21 | Incr. | 5856.94 | 0.68 | 0.73 |
| Analyte 22 | Incr. | 58.83 | 1.00 | 0.57 |
| Analyte 23 | Incr. | 4556.97 | 0.71 | 0.63 |
| Analyte 24 | Incr. | 224.83 | 0.41 | 0.68 |
| Analyte 25 | Incr. | 10080.59 | 0.89 | 0.53 |
| Analyte 26 | Incr. | 13.74 | 0.50 | 0.66 |
| Analyte 27 | Incr. | 2.64 | 0.43 | 0.77 |
| Analyte 28 | Decr. | 11678.95 | 0.69 | 0.52 |
| Analyte 29 | Incr. | 1.70 | 1.00 | 0.66 |
| Analyte 30 | Incr. | 1283.89 | 1.00 | 0.54 |
| Analyte 31 | Incr. | 10.96 | 1.00 | 0.50 |
| Analyte 32 | Decr. | 18882.79 | 1.00 | 0.58 |
| Analyte 33 | Decr. | 0.42 | 1.00 | 0.62 |
| Analyte 34 | Decr. | 3.99 | 0.96 | 0.52 |
| Analyte 35 | Decr. | 4950.62 | 0.41 | 0.64 |
| Analyte 36 | Incr. | 45.17 | 1.00 | 0.52 |
| Analyte 37 | Incr. | 126.85 | 0.71 | 0.58 |
| Analyte 38 | Decr. | 686.75 | 0.47 | 0.73 |

TABLE 2
Data Optimized and Tested with Different Criteria Sets

| | Test Set | | | | | | | | |
|---|---|---|---|---|---|---|---|---|---|
| | Stroke vs. NHD & Mimics | | | Stroke vs. NHD | | | Stroke vs. Mimics | | |
| Optimization Criteria | Ave Area | Ave Sens | Ave Spec | Ave Area | Ave Sens | Ave Spec | Ave Area | Ave Sens | Ave Spec |
| “Mimics” | 0.818 | 0.635 | 0.211 | 0.806 | 0.628 | 0.152 | 0.985 | 0.973 | 0.978 |
| “NHD & Mimics” | 0.982 | 0.969 | 0.962 | 0.992 | 0.975 | 0.991 | 0.854 | 0.558 | 0.582 |
| “NHD & Mimics” and “Mimics” | 0.961 | 0.906 | 0.89 | 0.966 | 0.911 | 0.905 | 0.899 | 0.78 | 0.732 |
| NHD & Mimics and “Mimics” | 0.953 | 0.901 | 0.883 | 0.958 | 0.908 | 0.898 | 0.891 | 0.766 | 0.722 |

We claim:

1. A method of identifying a panel of markers for diagnosis of a disease or a condition, comprising:
a) calculating a panel response for each patient in a set of diseased patients and in a set of non-diseased patients, said panel response being a function of value of each of a plurality of markers in a panel of markers;
b) calculating a value for an objective function, said objective function being indicative of an effectiveness of the panel; and
c) iterating steps a) and b) by varying at least one of parameters relating to said panel response function and a sense of each marker to facilitate optimization of said objective function.

2. The method according to claim 1, wherein said objective function is a measure of an overlap of panel responses of diseased patients and panel responses of non-diseased patients.

3. The method according to claim 1, wherein said panel response is a function of value of an indicator for each of a plurality of markers in a panel of markers and a weighting coefficient for each marker, said indicator being a mapping, for each of said plurality of markers, of marker levels, said mapping being according to an indicator function; and
wherein said iterating includes varying at least one of said weighting coefficients, parameters relating to said indicator function, and a sense of each marker to facilitate optimization of said objective function.

4. The method according to claim 3, wherein each indicator has a first value for marker levels below a cutoff region and a second value for marker values above a cutoff region, said cutoff region being defined by a location and a length.

5. The method according to claim 4, wherein said parameters include said location of said cutoff region and said length of said cutoff region.

6. The method according to claim 4, wherein said length of said cutoff region is zero.

7. The method according to claim 4, wherein said length of said cutoff region is greater than zero.

8. The method according to claim 7, wherein said indicators have values between said first value and said second value for marker levels within said cutoff region.

9. The method according to claim 8, wherein said indicators have values varying linearly from said first value to said second value across said cutoff region.

10. The method according to claim 8, wherein said indicators have values varying non-linearly from said first value to said second value across said cutoff region.

11. The method according to claim 10, wherein said non-linear variation is indicative of an error function of a distribution of marker values of diseased patients and an error function of a distribution of marker values of non-diseased patients within said cutoff region.

12. The method according to claim 3, wherein said calculating a panel response includes calculating, for each patient, ΣwᵢIᵢ, where w is a weighting coefficient for a marker i, I is the indicator value for the marker i, and Σ is a summation over all of said plurality of markers.

13. The method according to claim 1, wherein said calculating a value for an objective function includes generating a receiver operating characteristic (ROC) curve for said panel response, said ROC curve being indicative of a sensitivity of said panel response as a function of one minus a specificity of said panel response.

14. The method according to claim 13, wherein said objective function is associated with an area under said ROC curve.

15. The method according to claim 13, wherein said objective function is associated with a knee of said ROC curve.

16. The method according to claim 13, wherein said objective function is associated with a sensitivity at a selected specificity level.

17. The method according to claim 13, wherein said objective function is associated with a specificity at a selected sensitivity level.

18. The method according to claim 13, wherein said objective function is associated with two or more of an area under said ROC curve, a knee of said ROC curve, a sensitivity at a selected specificity level, and a specificity at a selected sensitivity level.

19. The method according to claim 13, wherein said iterating constrains at least one of an area under said ROC curve, a knee of said ROC curve, a sensitivity at a selected specificity level, and a specificity at a selected sensitivity level to be above about 0.9.

20. The method according to claim 1, further comprising:
d) removing at least one of said markers from said panel;
e) calculating a value of said objective function; and
f) determining a contribution of said at least one of said markers to said objective function based on a result of step e).

21. The method according to claim 20, further comprising:
g) repeating steps d) through f) by removing a different at least one of said markers from said panel; and
h) eliminating a marker from said panel of markers in accordance with said contribution of said marker to said objective function.

22. The method according to claim 1, further comprising:
d) removing at least one of said markers from said panel;
e) iterating steps a) and b) by varying parameters relating to said panel response function to facilitate optimization of said objective function; and
f) determining a contribution of said at least one of said markers to said objective function based on a result of step e).

23. The method according to claim 22, further comprising:
g) repeating steps d) through f) by removing a different at least one of said markers from said panel; and
h) eliminating a marker from said panel of markers in accordance with said contribution of said marker to said objective function.

24. A system for identifying a panel of markers for diagnosis of a disease or a condition, comprising:
means for calculating a panel response for each patient in a set of diseased patients and in a set of non-diseased patients, said panel response being a function of value of each of a plurality of markers in a panel of markers;
means for calculating a value for an objective function, said objective function being indicative of an effectiveness of said panel; and
means for iteratively activating said means for calculating a panel response and said means for calculating a value for an objective function, by varying at least one of parameters relating to said panel response function and a sense of each marker to facilitate optimization of said objective function.

25. The system according to claim 24, wherein said objective function is a measure of an overlap of panel responses of diseased patients and panel responses of non-diseased patients.

26. The system according to claim 24, wherein said panel response is a function of value of an indicator for each of a plurality of markers in a panel of markers and a weighting coefficient for each marker, said indicator being a mapping, for each of said plurality of markers, of marker levels, said mapping being according to an indicator function; and
wherein said means for iteratively activating is adapted to vary at least one of said weighting coefficients, parameters relating to said indicator function, and a sense of each marker to facilitate optimization of said objective function.

27. The system according to claim 26, wherein each indicator has a first value for marker levels below a cutoff region and a second value for marker values above a cutoff region, said cutoff region being defined by a location and a length.

28. The method according to claim 27, wherein said parameters include said location of said cutoff region and said length of said cutoff region.

29. The system according to claim 27, wherein said length of said cutoff region is zero.

30. The system according to claim 27, wherein said length of said cutoff region is greater than zero.

31. The system according to claim 30, wherein said indicators have values between said first value and said second value for marker levels within said cutoff region.

32. The system according to claim 31, wherein said indicators have values varying linearly from said first value to said second value across said cutoff region.

33. The system according to claim 32, wherein said indicators have values varying non-linearly from said first value to said second value across said cutoff region.

34. The system according to claim 33, wherein said non-linear variation is indicative of an error function of a distribution of marker values of diseased patients and an error function of a distribution of marker values of non-diseased patients within said cutoff region.

35. The system according to claim 26, wherein said means for calculating a panel response is adapted to calculate, for each patient, ΣwᵢIᵢ, where w is a weighting coefficient for a marker i, I is the indicator value for the marker I, and Σ is a summation over all of said plurality of markers.

36. The system according to claim 24, wherein said means for calculating a value for an objective function is adapted to generate a receiver operating characteristic (ROC) curve for said panel response, said ROC curve being indicative of a sensitivity of said panel response as a function of one minus a specificity of said panel response.

37. The system according to claim 36, wherein said objective function is associated with an area under said ROC curve.

38. The system according to claim 36, wherein said objective function is associated with a knee of said ROC curve.

39. The system according to claim 38, wherein said objective function is associated with a sensitivity at a selected specificity level.

40. The system according to claim 36, wherein said objective function is associated with a specificity at a selected sensitivity level.

41. The system according to claim 36, wherein said objective function is associated with two or more of an area under said ROC curve, a knee of said ROC curve, a sensitivity at a selected specificity level, and a specificity at a selected sensitivity level.

42. The system according to claim 36, wherein said means for iteratively activating is adapted to constrain at least one of an area under said ROC curve, a knee of said ROC curve, a sensitivity at a selected specificity level, and a specificity at a selected sensitivity level to be above about 0.9.

43. The system according to claim 24, further comprising:
means for determining a contribution of said at least one of said markers to said objective function, said means for determining being adapted to remove at least one of said markers from said panel and to activate said means for calculating a value for an objective function.

44. The system according to claim 43, further comprising:
means for eliminating a marker from said panel of markers in accordance with said contribution of said marker to said objective function, said means for eliminating being adapted to activate said means for determining a contribution by removing a different at least one of said markers from said panel.

45. The system according to claim 24, further comprising:
means for determining a contribution of said at least one of said markers to said objective function, said means for determining being adapted to remove at least one of said markers from said panel and to iteratively activate said means for calculating a panel response and said means for calculating a value for an objective function, by varying parameters relating to said panel response function to facilitate optimization of said objective function.

46. The system according to claim 45, further comprising:
means for eliminating a marker from said panel of markers in accordance with said contribution of said marker to said objective function, said means for eliminating being adapted to activate said means for determining a contribution by removing a different at least one of said markers from said panel.

47. A program product, comprising machine readable program code for causing a machine to perform following method steps:
a) calculating a panel response for each patient in a set of diseased patients and in a set of non-diseased patients, said panel response being a function of value of each of a plurality of markers in a panel of markers;
b) calculating a value for an objective function, said objective function being indicative of an effectiveness of said panel; and
c) iterating steps a) and b) by varying at least one of parameters relating to said panel response function and a sense of each marker to facilitate optimization of said objective function.

48. The program product according to claim 47, wherein said objective function is a measure of an overlap of panel responses of diseased patients and panel responses of non-diseased patients.

49. The program product according to claim 47, wherein said panel response is a function of value of an indicator for each of a plurality of markers in a panel of markers and a weighting coefficient for each marker, said indicator being a mapping, for each of said plurality of markers, of marker levels, said mapping being according to an indicator function; and
wherein said iterating includes varying at least one of said weighting coefficients, parameters relating to said indicator function, and a sense of each marker to facilitate optimization of said objective function.

50. The program product according to claim 49, wherein each indicator has a first value for marker levels below a cutoff region and a second value for marker values above a cutoff region, said cutoff region being defined by a location and a length.

51. The program product according to claim 50, wherein said parameters include said location of said cutoff region and said length of said cutoff region.

52. The program product according to claim 50, wherein said length of said cutoff region is zero.

53. The program product according to claim 50, wherein said length of said cutoff region is greater than zero.

54. The program product according to claim 53, wherein said indicators have values between said first value and said second value for marker levels within said cutoff region.

55. The program product according to claim 54, wherein said indicators have values varying linearly from said first value to said second value across said cutoff region.

56. The program product according to claim 54, wherein said indicators have values varying non-linearly from said first value to said second value across said cutoff region.

57. The program product according to claim 56, wherein said non-linear variation is indicative of an error function of a distribution of marker values of diseased patients and an error function of a distribution of marker values of non-diseased patients within said cutoff region.

58. The program product according to claim 49, wherein said calculating a panel response includes calculating, for each patient, ΣwᵢIᵢ, where w is a weighting coefficient for a marker i, I is the indicator value for the marker i, and Σ is a summation over all of said plurality of markers.

59. The program product according to claim 47, wherein said calculating a value for an objective function includes generating a receiver operating characteristic (ROC) curve for said panel response, said ROC curve being indicative of a sensitivity of said panel response as a function of one minus a specificity of said panel response.

60. The program product according to claim 59, wherein said objective function is associated with an area under said ROC curve.

61. The program product according to claim 59, wherein said objective function is associated with a knee of said ROC curve.

62. The program product according to claim 59, wherein said objective function is associated with a sensitivity at a selected specificity level.

63. The program product according to claim 59, wherein said objective function is associated with a specificity at a selected sensitivity level.

64. The program product according to claim 59, wherein said objective function is associated with two or more of an area under said ROC curve, a knee of said ROC curve, a sensitivity at a selected specificity level, and a specificity at a selected sensitivity level.

65. The program product according to claim 59, wherein said iterating constrains at least one of an area under said ROC curve, a knee of said ROC curve, a sensitivity at a selected specificity level, and a specificity at a selected sensitivity level to be above about 0.9.

66. The program product according to claim 47, further comprising machine readable program code for causing a machine to perform following method steps:
d) removing at least one of said markers from said panel;
e) calculating a value of said objective function; and
f) determining a contribution of said at least one of said markers to said objective function based on a result of step e).

67. The program product according to claim 66, further comprising machine readable program code for causing a machine to perform following method steps:
g) repeating steps d) through f) by removing a different at least one of said markers from said panel; and
h) eliminating a marker from said panel of markers in accordance with said contribution of said marker to said objective function.

68. The program product according to claim 47, further comprising machine readable program code for causing a machine to perform following method steps:
d) removing at least one of said markers from said panel;
e) iterating steps a) and b) by varying parameters relating to said panel response function to facilitate optimization of said objective function; and
f) determining a contribution of said at least one of said markers to said objective function based on a result of step e).

69. The program product according to claim 68, further comprising machine readable program code for causing a machine to perform following method steps:
g) repeating steps d) through f) by removing a different at least one of said markers from said panel; and
h) eliminating a marker from said panel of markers in accordance with said contribution of said marker to said objective function.

70. The program product according to claim 47, wherein said machine readable code is embedded in a portable meter.

71. The program product according to claim 70, wherein said portable meter is a fluorometer.

72. The program product according to claim 70, wherein said portable meter is a reflectometer.

73. The program product according to claim 47, wherein said machine readable code is embedded in a computer.

74. The program product according to claim 73, wherein said computer is a portable computer.

75. The program product according to claim 73, wherein said computer is adapted to be accessed through a network.

76. The program product according to claim 75, wherein said network is the Internet.

77. The program product according to claim 73, wherein said computer is adapted to be coupled to an analyzer.

78. The program product according to claim 77, wherein said analyzer is an immunoassay analyzer.

79. The program product according to claim 77, wherein said analyzer is a single nucleotide polymorphism detector.

80. The program product according to claim 77, wherein said analyzer is adapted to sort and count similar and different particles and cells.

81. A method of identifying a panel of markers for diagnosis of a disease or a condition, comprising:
a) selecting a panel of markers, said panel including a plurality of markers measured in a set of diseased patients and a set of non-diseased patients;
b) defining a cutoff region of marker levels for each of said plurality of markers, said cutoff region having a location and a length;
c) selecting a weighting coefficient for each of said plurality of markers;
d) mapping, for each of said plurality of markers, marker levels to an indicator, each of said indicators having a first value for marker levels below said cutoff region and a second value for marker levels above said cutoff region;
e) calculating a panel response for each patient in said set of diseased patients and in said set of non-diseased patients, said panel response being a function of value of said indicator for each marker and said weighting coefficient for each marker;
f) calculating a value for an objective function, said objective function being indicative of an effectiveness of said panel; and
g) iterating steps e) and f) by varying at least one of said location of said cutoff region, said length of said cutoff region, said weighting coefficients, and a sense of each marker to facilitate optimization of said objective function.

82. The method according to claim 81, wherein said objective function is a measure of an overlap of panel responses of diseased patients and panel responses of non-diseased patients.

83. The method according to claim 81, wherein said length of said cutoff region is zero.

84. The method according to claim 81, wherein said length of said cutoff region is greater than zero.

85. The method according to claim 84, wherein said indicators have values between said first value and said second value for marker levels within said cutoff region.

86. The method according to claim 85, wherein said indicators have values varying linearly from said first value to said second value across said cutoff region.

87. The method according to claim 85, wherein said indicators have values varying non-linearly from said first value to said second value across said cutoff region.

88. The method according to claim 87, wherein said non-linear variation is indicative of an error function of a distribution of marker values of diseased patients and an error function of a distribution of marker values of non-diseased patients within said cutoff region.

89. The method according to claim 81, wherein said calculating a panel response includes calculating, for each patient, ΣwᵢIᵢ, where w is a weighting coefficient for a marker i, I is the indicator value for the marker I, and Σ is a summation over all of said plurality of markers.

90. The method according to claim 81, wherein said calculating a value for an objective function includes generating a receiver operating characteristic (ROC) curve for said panel response, said ROC curve being indicative of a sensitivity of said panel response as a function of one minus a specificity of said panel response.

91. The method according to claim 90, wherein said objective function is associated with an area under said ROC curve.

92. The method according to claim 90, wherein said objective function is associated with a knee of said ROC curve.

93. The method according to claim 90, wherein said objective function is associated with a sensitivity at a selected specificity level.

94. The method according to claim 90, wherein said objective function is associated with a specificity at a selected sensitivity level.

95. The method according to claim 90, wherein said objective function is associated with two or more of an area under said ROC curve, a knee of said ROC curve, a sensitivity at a selected specificity level, and a specificity at a selected sensitivity level.

96. The method according to claim 81, further comprising:
h) setting said weighting coefficient of at least one of said markers to approximately zero;
i) calculating a value of said objective function with remaining weighting coefficients; and
j) determining a contribution of said at least one of said markers to said objective function.

97. The method according to claim 96, further comprising:
k) repeating steps h) through j) by setting said weighting coefficient of a different at least one of said markers to approximately zero; and
l) eliminating a marker from said panel of markers in accordance with said contribution of said marker to said objective function.

98. A method of identifying a panel of markers for diagnosis of a disease or a condition, comprising:
a) identifying a cutoff region for each of a plurality of markers, said cutoff region being substantially centered about an overlap region of marker values for a set of diseased patients and a set of non-diseased patients, said cutoff region having a location and a length;
b) determining an effectiveness value of each of said plurality of markers in distinguishing said set of diseased patients from said set of non-diseased patients; and
c) defining a panel response as a function of said effectiveness value of each marker and a measured level of each marker.

99. The method according to claim 98, wherein said cutoff region has a length of zero.

100. The method according to claim 98, wherein said cutoff region has a non-zero length.

101. The method according to claim 98, wherein said effectiveness value of each marker is represented by an area under a ROC curve.

102. The method according to claim 3, wherein said indicator function is monotonic with marker value.

103. The method according to claim 102, wherein said indicator function is one of the group consisting of: a ramp function, a step function, and a sigmoid function.

104. The method according to claim 3, wherein said indicator function is adapted to localize a marker value.

105. The method according to claim 104, wherein said indicator function is one of the group consisting of: a triangle, a square, and Gaussian.

106. The method according to claim 1, wherein at least one of said plurality of markers is a derived marker.

107. The method according to claim 106, wherein said derived marker is the ratio of two other markers.

108. The method according to claim 1, wherein said iterating includes using a downhill simplex method.

109. The method according to claim 108, wherein said iterating further includes simulated annealing.

110. The method according to claim 109, wherein said simulated annealing includes performing a statistically sufficient number of optimizations to evaluate a most common solution.

111. The method according to claim 1, wherein said optimization is adapted to provide a stable solution.

112. The method according to claim 111, wherein said adaptation includes varying the marker values by a random percentage.

113. The method according to claim 111, wherein said adaptation includes varying one or more parameters of said panel function.

114. The method according to claim 111, wherein said adaptation includes generating a seed simplex about a minimum.

115. The method according to claim 111, wherein said adaptation includes increasing an annealing temperature until an achieved solution is not recovered.

116. The method according to claim 20, wherein said removing includes setting a weighting coefficient of said at least one of said markers to approximately zero.

117. The system according to claim 26, wherein said indicator function is monotonic with marker value.

118. The system according to claim 117, wherein said indicator function is one of the group consisting of: a ramp function, a step function, and a sigmoid function.

119. The system according to claim 26, wherein said indicator function is adapted to localize a marker value.

120. The system according to claim 119, wherein said indicator function is one of the group consisting of: a triangle, a square, and Gaussian.

121. The system according to claim 43, wherein said removing includes setting a weighting coefficient of said at least one of said markers to approximately zero.

122. The program product according to claim 49, wherein said indicator function is monotonic with marker value.

123. The program product according to claim 49, wherein said indicator function is one of the group consisting of: a ramp function, a step function, and a sigmoid function.

124. The program product according to claim 49, wherein said indicator function is adapted to localize a marker value.

125. The program product according to claim 49, wherein said indicator function is one of the group consisting of: a triangle, a square, and Gaussian.

126. The method according to claim 106, wherein said derived marker is indicative of the change in another marker over time.

127. The method according to claim 106, wherein said derived marker is indicative of the change in said panel response over time.

128. The program product according to claim 66, wherein said removing includes setting a weighting coefficient of said at least one of said markers to approximately zero.

129. The method according to claim 1, wherein said optimization is adapted to simultaneously at least one of optimize and constrain a plurality of objective functions calculated from a plurality of groups of data.

130. The system according to claim 24, wherein said means for iteratively activating is adapted to simultaneously at least one of optimize and constrain a plurality of objective functions calculated from a plurality of groups of data.

131. The program product according to claim 47, wherein said optimization is adapted to simultaneously at least one of optimize and constrain a plurality of objective functions calculated from a plurality of groups of data.
